(19) United States
(12) Patent Application Publication
(10) Pub. No.: US 2024/0415891 A1
(43) Pub. Date: Dec. 19, 2024

(54) ACTIVATION RESPONSIVE PROMOTERS AND USES THEREOF

(71) Applicant: Senti Biosciences, Inc., South San Francisco, CA (US)

(72) Inventors: Michelle Elizabeth Hung, South San Francisco, CA (US); Assen Boyanov Roguev, San Francisco, CA (US)

(21) Appl. No.: 18/743,045

(22) Filed: Jun. 13, 2024

Related U.S. Application Data
(63) Continuation of application No. PCT/US2022/081658, filed on Dec. 15, 2022.
(60) Provisional application No. 63/352,995, filed on Jun. 16, 2022.
(60) Provisional application No. 63/289,983, filed on Dec. 15, 2021.

(51) Int. Cl. A61K 35/17; A61K 39/00; A61K 48/00; C12N 15/85

(52) U.S. Cl. CPC A61K 35/17; A61K 39/4611; A61K 39/4613; A61K 39/4631; A61K 39/464421; A61K 48/005; C12N 15/85

(57) ABSTRACT

Described herein are compositions and methods for regulating expression of effector molecules using engineered CCL3 promoters. Immunoresponsive cells comprising the same are also described.

### CROSS REFERENCE TO RELATED APPLICATIONS

This application is a continuation of International Application No. PCT/US2022/081658, filed Dec. 15, 2022, which claims the benefit of and priority to U.S. Provisional Patent Application No. 63/289,983, filed Dec. 15, 2021, and to U.S. Provisional Patent Application No. 63/352,995, filed Jun. 16, 2022, each of which are hereby incorporated by reference in their entirety for all purposes.

### SEQUENCE LISTING

The instant application contains a Sequence Listing XML which has been submitted electronically and is hereby incorporated by reference in its entirety. Said XML copy, created on May 2, 2023, is named STB-035USWOC1.xml, and is 411,766 bytes in size.

### BACKGROUND

Cell-based therapy platforms provide promising avenues for treating a variety of diseases. One such promising platform is CAR-T based therapies in the treatment of cancer. Given their promise, improvements in cell-based therapies are needed. An active area of exploration is engineering cell-based therapies to produce and/or secrete effector molecules such as cytokines, a process referred to as armoring, that enhance the cell-based therapy. For example, unarmored CAR-T therapies have poor efficacy in solid tumors and armoring can impact the entire cancer immunity cycle and boost the activity of CAR-T. However, uncontrolled or unregulated armoring strategies can have negative impacts on treatment, such as off-target effects and toxicity in subjects. Thus, additional methods of controlling and regulating the armoring of cell-based therapies, such as regulating production and/or secretion of payload effector molecules, are required.

### SUMMARY

One embodiment of the present disclosure provides an engineered CCL3 promoter comprising an ablation of at least one nucleotide motif, wherein the ablation increases inducibility of the engineered CCL3 promoter in the presence of an immune cell activation signal, as compared to inducibility of a wild-type CCL3 promoter in the presence of the same immune cell activation signal.

In one aspect, provided herein is an engineered CCL3 promoter comprising an ablation of at least one nucleotide motif, wherein the ablation increases inducibility of the engineered CCL3 promoter in the presence of an immune cell activation signal, as compared to inducibility of a wild-type CCL3 promoter in the presence of the same immune cell activation signal;
optionally wherein the wild-type CCL3 promoter comprises the nucleotide sequence of SEQ ID NO: 132, optionally wherein the at least one nucleotide motif comprises a motif having a sequence within the nucleotide sequence of SEQ ID NO: 132;
optionally wherein the motif comprises a sequence selected from the group consisting of: position 566 to position 576 of SEQ ID NO: 132, position 674 to position 695 of SEQ ID NO: 132, position 820 to position 832 of SEQ ID NO: 132, position 1089 to position 1105 of SEQ ID NO: 132, position 1127 to position 1141 of SEQ ID NO: 132, position 1184 to position 1199 of SEQ ID NO: 132, position 1475 to position 1500 of SEQ ID NO: 132, position 1534 to position 1544 of SEQ ID NO: 132, position 1553 to position 1595 of SEQ ID NO: 132, position 1634 to position 1674 of SEQ ID NO: 132, position 1681 to position 1692 of SEQ ID NO: 132, and position 1982 to position 1998 of SEQ ID NO: 132;
optionally wherein the ablation comprises a substitution or deletion of one or more nucleotides of the at least one nucleotide motif, optionally wherein the at least one nucleotide motif comprises a motif having a sequence within the nucleotide sequence of SEQ ID NO: 132;
optionally wherein the motif corresponds to position 566 to position 576 of SEQ ID NO: 132, optionally wherein the ablation comprises a nucleotide substitution comprising the sequence GTACCAGAATA (SEQ ID NO:191) from position 566 to position 576 of SEQ ID NO:132, optionally wherein the ablation comprises nucleotide deletions of position 566 to position 576 of SEQ ID NO:132, optionally wherein the at least one nucleotide motif comprises a motif having a sequence within the nucleotide sequence of SEQ ID NO: 132, optionally wherein the motif corresponds to position 674 to position 695 of SEQ ID NO:132, optionally wherein the ablation comprises a nucleotide substitution comprising the sequence TATATACCAGCGAGTTCGATAA (SEQ ID NO:193) from position 674 to position 695 of SEQ ID NO:132, optionally wherein the ablation comprises nucleotide deletions of position 674 to position 695 of SEQ ID NO:132, optionally wherein the at least one nucleotide motif comprises a motif having a sequence within the nucleotide sequence of SEQ ID NO: 132, optionally wherein the motif corresponds to position 820 to position 832 of SEQ ID NO:132, optionally wherein the ablation comprises a nucleotide substitution comprising the sequence ACGAAGCAATACT (SEQ ID NO:195) from position 820 to position 832 of SEQ ID NO:132, optionally wherein the ablation comprises nucleotide deletions of position 820 to position 832 of SEQ ID NO:132, optionally wherein the at least one nucleotide motif comprises a motif having a sequence within the nucleotide sequence of SEQ ID NO:132, optionally wherein the motif corresponds to position 1089 to position 1105 of SEQ ID NO:132, optionally wherein the ablation comprises a nucleotide substitution comprising the sequence TCTGTATAAAGCTCGTA (SEQ ID NO:199) from position 1089 to position 1105 of SEQ ID NO:132, optionally wherein the ablation comprises nucleotide deletions of position 1089 to position 1105 of SEQ ID NO:132, optionally wherein the at least one nucleotide motif comprises a motif having a sequence within the nucleotide sequence of SEQ ID NO: 132, optionally wherein the motif corresponds to position 1127 to position 1141 of SEQ ID NO:132, optionally wherein the ablation comprises a nucleotide substitution comprising the sequence CCATAGTGAGGAAAT (SEQ ID NO:201) from position 1127 to position 1141 of SEQ ID NO:132, optionally wherein the ablation comprises nucleotide deletions of position 1127 to position 1141 of SEQ ID NO:132, optionally wherein the at least one nucleotide motif comprises a motif having a sequence within the nucleotide sequence of SEQ ID NO:132, optionally wherein the motif corresponds to position 1184 to position 1199 of SEQ ID NO:132, optionally wherein the ablation comprises a nucleotide substitution comprising the sequence GTTAAGCATACTAAAC (SEQ ID NO:203) from position 1184 to position 1199 of SEQ ID NO:132, optionally wherein the ablation comprises nucleotide deletions of position 1184 to position 1199 of SEQ ID NO:132, optionally wherein the at least one nucleotide motif comprises a motif having a sequence within the nucleotide sequence of SEQ ID NO: 132, wherein the motif corresponds to position 1475 to position 1500 of SEQ ID NO:132, optionally wherein the ablation comprises a nucleotide substitution comprising the sequence CCGATCTCTAGTTAAGTTAGCTGTAT (SEQ ID NO:217) from position 1475 to position 1500 of SEQ ID NO:132, optionally wherein the ablation comprises nucleotide deletions of position 1475 to position 1500 of SEQ ID NO:132, optionally wherein the at least one nucleotide motif comprises a motif having a sequence within the nucleotide sequence of SEQ ID NO:132, optionally wherein the motif corresponds to position 1534 to position 1544 of SEQ ID NO:132, optionally wherein the ablation comprises a nucleotide substitution comprising the sequence GTAGAACTCTT (SEQ ID NO:219) from position 1534 to position 1544 of SEQ ID NO:132, optionally wherein the ablation comprises nucleotide deletions of position 1534 to position 1544 of SEQ ID NO:132, optionally wherein the at least one nucleotide motif comprises a motif having a sequence within the nucleotide sequence of SEQ ID NO:132, optionally wherein the motif corresponds to position 1553 to position 1595 of SEQ ID NO:132, optionally wherein the ablation comprises a nucleotide substitution comprising the sequence AATACCTTGGTGGAGCTCATCTAATATCTTCATATTACCTCCA (SEQ ID NO:221) from position 1553 to position 1595 of SEQ ID NO:132, optionally wherein the ablation comprises nucleotide deletions of position 1553 to position 1595 of SEQ ID NO:132, optionally wherein the at least one nucleotide motif comprises a motif having a sequence within the nucleotide sequence of SEQ ID NO: 132, optionally wherein the motif corresponds to position 1634 to position 1645 of SEQ ID NO:132, optionally wherein the ablation comprises a nucleotide substitution comprising the sequence CGATTGAACAAA (SEQ ID NO:223) from position 1634 to position 1645 of SEQ ID NO:132, optionally wherein the ablation comprises nucleotide deletions of position 1634 to position 1645 of SEQ ID NO:132, optionally wherein the at least one nucleotide motif comprises a motif having a sequence within the nucleotide sequence of SEQ ID NO: 132, optionally wherein the motif corresponds to position 1663 to position 1674 of SEQ ID NO:132, optionally wherein the ablation comprises a nucleotide substitution comprising the sequence TATACCTTGGTT (SEQ ID NO:225) from position 1663 to position 1674 of SEQ ID NO:132, optionally wherein the ablation comprises nucleotide deletions of position 1663 to position 1674 of SEQ ID NO:132, optionally wherein the at least one nucleotide motif comprises a motif having a sequence within the nucleotide sequence of SEQ ID NO:132, wherein the motif corresponds to position 1681 to position 1692 of SEQ ID NO:132, optionally wherein the ablation comprises a nucleotide substitution comprising the sequence ATTGCGTCAATT (SEQ ID NO:227) from position 1681 to position 1692 of SEQ ID NO:132, optionally wherein the engineered CCL3 promoter comprises the polynucleotide of SEQ ID NO: 4;
optionally wherein the ablation comprises nucleotide deletions of position 1681 to position 1692 of SEQ ID NO:132, optionally wherein the at least one nucleotide motif comprises a motif having a sequence within the nucleotide sequence of SEQ ID NO: 132, optionally wherein the motif corresponds to position 1982 to position 1998 of SEQ ID NO:132, optionally wherein the ablation comprises a nucleotide substitution comprising the sequence GGAAGTAGCTTGTTTAA (SEQ ID NO:241) from position 1982 to position 1998 of SEQ ID NO:132, optionally wherein the ablation comprises nucleotide deletions of position 1982 to position 1998 of SEQ ID NO:132;
optionally wherein the ablation comprises an ablation of at least two nucleotide motifs, at least three nucleotide motifs, at least four nucleotide motifs, at least five nucleotide motifs, or at least six nucleotide motifs;
optionally wherein the at least six nucleotide motifs comprise: a nucleotide motif corresponding to positions 566-576 of SEQ ID NO:132, a nucleotide motif corresponding to positions 674-695 of SEQ ID NO:132, a nucleotide motif corresponding to positions 820-832 of SEQ ID NO:132, a nucleotide motif corresponding to positions 1089-1105 of SEQ ID NO:132, a nucleotide motif corresponding to positions 1127-1141 of SEQ ID NO:132, and a nucleotide motif corresponding to positions 1184-1199 of SEQ ID NO:132,
optionally wherein the ablation of the at least six nucleotide motifs comprise a deletion corresponding to position 566 to position 1199 of SEQ ID NO:132, optionally wherein the ablation of the at least six nucleotide motifs comprise a deletion corresponding to position 550 to position 1259 of SEQ ID NO:132, optionally wherein the ablation further comprises an ablation of a nucleotide motif corresponding to positions 1553 to position 1595 of SEQ ID NO:132, optionally wherein the ablation of the nucleotide motif corresponding to positions 1553 to position 1595 of SEQ ID NO:132 comprises a deletion of the nucleotide motif, optionally wherein the ablation further comprises an ablation of a nucleotide motif corresponding to positions 1681 to position 1692 of SEQ ID NO:132, optionally wherein the ablation comprises a nucleotide substitution comprising the sequence ATTGCGTCAATT (SEQ ID NO:227) from position 1681 to position 1692 of SEQ ID NO:132, optionally wherein the engineered CCL3 promoter comprises the polynucleotide sequence of SEQ ID NO: 2;
optionally wherein the ablation of the nucleotide motif corresponding to positions 1681-1692 of SEQ ID NO:132 comprises a deletion of the nucleotide motif, optionally wherein the ablation further comprises an ablation of a nucleotide motif corresponding to positions 1475 to position 1500 of SEQ ID NO:132, optionally wherein the ablation of the nucleotide motif corresponding to positions 1475 to position 1500 of SEQ ID NO:132 comprises a deletion of the nucleotide motif, optionally wherein the engineered CCL3 promoter comprises the polynucleotide sequence of SEQ ID NO: 1,
optionally wherein the ablation comprises an ablation of at least nine nucleotide motifs, optionally wherein the at least nine nucleotide motifs comprise: a nucleotide motif corresponding to positions 566-576 of SEQ ID NO:132, a nucleotide motif corresponding to positions 674-695 of SEQ ID NO:132, a nucleotide motif corresponding to positions 820-832 of SEQ ID NO:132, a nucleotide motif corresponding to positions 1089-1105 of SEQ ID NO:132, a nucleotide motif corresponding to positions 1127-1141 of SEQ ID NO:132, a nucleotide motif corresponding to positions 1184-1199 of SEQ ID NO:132, a nucleotide motif corresponding to positions 1475-1500 of SEQ ID NO:132, a nucleotide motif corresponding to positions 1553-1595 of SEQ ID NO:132, and a nucleotide motif corresponding to positions 1681-1692 of SEQ ID NO:132, optionally wherein the ablation comprises a deletion of each of the at least nine nucleotide motifs, optionally wherein the engineered CCL3 promoter comprises the polynucleotide sequence of SEQ ID NO: 3,
optionally wherein the at least nine nucleotide motifs comprise: a nucleotide motif corresponding to positions 566-576 of SEQ ID NO:132, a nucleotide motif corresponding to positions 674-695 of SEQ ID NO:132, a nucleotide motif corresponding to positions 820-832 of SEQ ID NO:132, a nucleotide motif corresponding to positions 911-926 of SEQ ID NO:132, a nucleotide motif corresponding to positions 1089-1105 of SEQ ID NO:132, a nucleotide motif corresponding to positions 1127-1141 of SEQ ID NO:132, a nucleotide motif corresponding to positions 1184-1199 of SEQ ID NO:132, a nucleotide motif corresponding to positions 1211-1223 of SEQ ID NO:132, and a nucleotide motif corresponding to positions 1236-1248 of SEQ ID NO:132, optionally wherein the ablation of the at least nine nucleotide motifs comprises a deletion corresponding to positions 556 to position 1248 of SEQ ID NO:132, optionally wherein the ablation of the at least nine nucleotide motifs comprises a deletion corresponding to positions 550 to position 1250 of SEQ ID NO:132, optionally wherein the ablation further comprises an ablation of a nucleotide motif corresponding to positions 1425 to position 1445 of SEQ ID NO: 132, optionally wherein the ablation of the nucleotide motif corresponding to positions 1425-1445 of SEQ ID NO:132 comprises a deletion of the nucleotide motif, optionally wherein the ablation further comprises an ablation of a nucleotide motif corresponding to positions 1475 to position 1500 of SEQ ID NO: 132, optionally wherein the ablation of the nucleotide motif corresponding to positions 1475-1500 of SEQ ID NO:132 comprises a deletion of the nucleotide motif, optionally wherein the ablation further comprises an ablation of a nucleotide motif corresponding to positions 1553 to position 1595 of SEQ ID NO: 132, optionally wherein the ablation of the nucleotide motif corresponding to positions 1553-1595 of SEQ ID NO:132 comprises a deletion of the nucleotide motif, optionally wherein the ablation further comprises an ablation of a nucleotide motif corresponding to positions 1663 to position 1679 of SEQ ID NO: 132, optionally wherein the ablation of the nucleotide motif corresponding to positions 1663 to position 1679 of SEQ ID NO:132 comprises a deletion of the nucleotide motif, optionally wherein the at least one nucleotide motif comprises a motif having a sequence within the nucleotide sequence of SEQ ID NO:132, optionally wherein the motif corresponds to position 1681 to position 1692 of SEQ ID NO:132, optionally wherein the ablation comprises a nucleotide substitution comprising the sequence ATTGCGTCAATT (SEQ ID NO:227) from position 1681 to position 1692 of SEQ ID NO:132, optionally wherein the CCL3 promoter comprises SEQ ID NO: 242,
optionally wherein the ablation comprises a deletion of at least two nucleotide motifs according to position 1425 to position 1500 of SEQ ID NO: 132, optionally wherein the ablation further comprises an ablation of a nucleotide motif corresponding to positions 1553 to position 1595 of SEQ ID NO: 132, optionally wherein the ablation of the nucleotide motif corresponding to positions 1553-1595 of SEQ ID NO:132 comprises a deletion of the nucleotide motif, optionally wherein the ablation further comprises an ablation of a nucleotide motif corresponding to positions 1663 to position 1679 of SEQ ID NO: 132, optionally wherein the ablation of the nucleotide motif corresponding to positions 1663-1679 of SEQ ID NO:132 comprises a deletion of the nucleotide motif, optionally wherein the at least one nucleotide motif comprises a motif having a sequence within the nucleotide sequence of SEQ ID NO:132, optionally wherein the motif corresponds to position 1681 to position 1692 of SEQ ID NO:132, optionally wherein the ablation comprises a nucleotide substitution comprising the sequence ATTGCGTCAATT (SEQ ID NO:227) from position 1681 to position 1692 of SEQ ID NO:132, optionally wherein the ablation further comprises a deletion corresponding to positions 1750 to position 1818 of SEQ ID NO:132, optionally wherein the CCL3 promoter comprises SEQ ID NO: 243, optionally wherein the ablation further comprises a deletion corresponding to positions 1867 to position 2000 of SEQ ID NO:132, optionally wherein the CCL3 promoter comprises SEQ ID NO: 244, optionally wherein the ablation further comprises a deletion to positions 1663 to position 1692 of SEQ ID NO:132, optionally wherein the ablation further comprises a deletion corresponding to positions 1750 to position 1818 of SEQ ID NO:132, optionally wherein the ablation further comprises a deletion corresponding to positions 1867 to position 2000 of SEQ ID NO:132, or optionally wherein the CCL3 promoter comprises the polynucleotide sequence of SEQ ID NO: 245.

In another aspect, provided herein is an engineered CCL3 promoter comprising at least one nucleotide motif,
optionally wherein the at least one nucleotide motif is selected from the group consisting of:
a) a nucleotide motif corresponding to positions 60-77 of SEQ ID NO:132,
b) a nucleotide motif corresponding to positions 92-111 of SEQ ID NO:132,
c) a nucleotide motif corresponding to positions 201-224 of SEQ ID NO:132,
d) a nucleotide motif corresponding to positions 231-243 of SEQ ID NO:132,
e) a nucleotide motif corresponding to positions 265-284 of SEQ ID NO:132,
f) a nucleotide motif corresponding to positions 307-324 of SEQ ID NO:132,
g) a nucleotide motif corresponding to positions 376-388 of SEQ ID NO:132,
h) a nucleotide motif corresponding to positions 452-475 of SEQ ID NO:132,
i) a nucleotide motif corresponding to positions 494-507 of SEQ ID NO:132,
k) a nucleotide motif corresponding to positions 533-549 of SEQ ID NO:132,
l) a nucleotide motif corresponding to positions 1260-1288 of SEQ ID NO:132,
m) a nucleotide motif corresponding to positions 1345-1363 of SEQ ID NO:132,
n) a nucleotide motif corresponding to positions 1534-1544 of SEQ ID NO:132,
o) a nucleotide motif corresponding to positions 1634-1645 of SEQ ID NO:132, and
p) a nucleotide motif corresponding to positions 1840-1861 of SEQ ID NO:132;
optionally wherein the engineered CCL3 promoter comprises at least fifteen nucleotide motifs comprising:
a) a nucleotide motif corresponding to positions 60-77 of SEQ ID NO:132,
b) a nucleotide motif corresponding to positions 92-111 of SEQ ID NO:132,
c) a nucleotide motif corresponding to positions 201-224 of SEQ ID NO:132,
d) a nucleotide motif corresponding to positions 231-243 of SEQ ID NO:132,
e) a nucleotide motif corresponding to positions 265-284 of SEQ ID NO:132,
f) a nucleotide motif corresponding to positions 307-324 of SEQ ID NO:132,
g) a nucleotide motif corresponding to positions 376-388 of SEQ ID NO:132,
h) a nucleotide motif corresponding to positions 452-475 of SEQ ID NO:132,
i) a nucleotide motif corresponding to positions 494-507 of SEQ ID NO:132,
j) a nucleotide motif corresponding to positions 533-549 of SEQ ID NO:132,
k) a nucleotide motif corresponding to positions 1260-1288 of SEQ ID NO:132,
l) a nucleotide motif corresponding to positions 1345-1363 of SEQ ID NO:132,
m) a nucleotide motif corresponding to positions 1534-1544 of SEQ ID NO:132,
n) a nucleotide motif corresponding to positions 1634-1645 of SEQ ID NO:132, and
o) a nucleotide motif corresponding to positions 1840-1861 of SEQ ID NO:132;
or optionally wherein the CCL3 promoter comprises the polynucleotide sequence of SEQ ID NO: 246.

In another aspect, provided herein is an engineered CCL3 promoter comprising a polynucleotide sequence selected from the group consisting of SEQ ID NOs: 1-4 and 242-246.

In another aspect, provided herein is a heterologous construct comprising the engineered CCL3 promoter according to any one of the above aspects or embodiments operably linked to a polynucleotide comprising a polynucleotide sequence encoding a polypeptide;
optionally wherein the polypeptide comprises at least one effector molecule,
optionally wherein the polypeptide comprises a first effector molecule and a second effector molecule,
optionally wherein the polynucleotide comprises a polynucleotide sequence encoding the first effector molecule, a linker polynucleotide sequence, and a polynucleotide sequence encoding the second effector molecule,
optionally wherein the linker polynucleotide sequence encodes one or more 2A ribosome skipping elements, optionally wherein the one or more 2A ribosome skipping elements comprise elements that are each selected from the group consisting of: P2A, T2A, E2A, and F2A,
optionally wherein the at least one effector molecule is selected from a therapeutic class, wherein the therapeutic class is selected from the group consisting of: a cytokine, a chemokine, a homing molecule, a growth factor, a co-activation molecule, a tumor microenvironment modifier, a receptor, a ligand, an antibody, a peptide, and an enzyme,
optionally wherein each of the at least one effector molecule comprises:
a) a cytokine, optionally wherein the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha;
b) a chemokine, optionally wherein the chemokine is selected from the group consisting of: CCL21a, CXCL10, CXCL11, CXCL13, a CXCL10-CXCL11 fusion protein, CCL19, CXCL9, and XCL1;
c) a homing molecule, optionally wherein the homing molecule is selected from the group consisting of: anti-integrin alpha4, beta7; anti-MAdCAM; CCR9; CXCR4; SDFI; MMP-2; CXCR1; CXCR7; CCR2; CCR4; and GPR15;
d) a growth factor, optionally wherein the growth factor is selected from the group consisting of: FLT3L and GM-CSF;
e) a co-activation molecule, optionally wherein the co-activation molecule is selected from the group consisting of: c-Jun, 4-1BBL and CD40L;
f) a tumor microenvironment modifier, optionally wherein the tumor microenvironment modifier is selected from the group consisting of: an adenosine deaminase, a TGFbeta inhibitor, an immune checkpoint inhibitor, a VEGF inhibitor, and an HPGE2;
optionally wherein each of the first effector molecule and the second effector molecule is from a separate therapeutic class;
and optionally wherein each of the at least one effector molecule is a human-derived effector molecule.

In another aspect, provided herein is a vector comprising the heterologous construct of any one of the above aspects or embodiments.

In another aspect, provided herein is a dual expression vector comprising the heterologous construct of the above aspect and a second construct comprising a polynucleotide sequence encoding an activating immune receptor, optionally wherein the activating immune receptor comprises an antigen recognizing receptor,
optionally wherein the antigen recognizing receptor comprises a T Cell Receptor (TCR), optionally wherein the TCR is an endogenous T cell receptor or an exogenous T cell receptor, and
optionally wherein the antigen recognizing receptor comprises a Chimeric Antigen Receptor (CAR).

In another aspect, provided herein is an immunoresponsive cell comprising the heterologous construct, the vector, or the dual expression vector of any one of the above aspects or embodiments,
optionally wherein the immunoresponsive cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell;
optionally wherein the immunoresponsive cell expresses an activating immune receptor, optionally wherein the activating immune receptor comprises an antigen recognizing receptor, optionally wherein the antigen recognizing receptor comprises a T Cell Receptor (TCR), optionally wherein the TCR is an endogenous T cell receptor or an exogenous T cell receptor, and optionally wherein the antigen recognizing receptor comprises a Chimeric Antigen Receptor (CAR);
optionally wherein the antigen recognizing receptor comprises an activating NK cell receptor, optionally wherein the activating NK cell receptor is selected from the group consisting of NKG2D, NKp30, NKp44, NKp46, and ITAM-containing Killer cell Ig-like receptors (KIRs);
optionally wherein the immunoresponsive cell comprises a heterologous construct encoding the antigen recognizing receptor; and
optionally wherein the immunoresponsive cell is autologous, or optionally wherein the immunoresponsive cell is allogeneic.

In another aspect, provided herein is a pharmaceutical composition comprising the engineered CCL3 promoter, the heterologous construct, the vector, the dual expression vector, the immunoresponsive cell, and a pharmaceutically acceptable carrier, pharmaceutically acceptable excipient, or a combination thereof.

In another aspect, provided herein is a method of increasing expression of a target gene, the method comprising use of the engineered CCL3 promoter, the heterologous construct, the vector, or the dual expression vector to increase expression of the target gene, optionally wherein the target gene is an immunomodulatory gene.

In another aspect, provided herein is a method of treating a subject in need thereof, the method comprising administering the engineered CCL3 promoter, the heterologous construct, the vector, the dual expression vector, the immunoresponsive cell, or the pharmaceutical composition of any one of the above aspects or embodiments.

In another aspect, provided herein is a method of stimulating a cell-mediated immune response in a subject, the method comprising administering to a subject the engineered CCL3 promoter, the heterologous construct, the vector, the dual expression vector, the immunoresponsive cell, or the pharmaceutical composition of any one of the above aspects or embodiments.

In another aspect, provided herein is a method of reducing tumor volume in a subject, the method comprising administering to a subject having a tumor a composition the engineered CCL3 promoter, the heterologous construct, the vector, the dual expression vector, the immunoresponsive cell, or the pharmaceutical composition of any one of the above aspects or embodiments.

In another aspect, provided herein is a method of providing an anti-tumor immunity in a subject, the method comprising administering to a subject in need thereof the engineered CCL3 promoter, the heterologous construct, the vector, the dual expression vector, the immunoresponsive cell, or the pharmaceutical composition of any one of the above aspects or embodiments.

In another aspect, provided herein is a method of treating a subject having cancer, the method comprising administering the engineered CCL3 promoter, the heterologous construct, the vector, the dual expression vector, the immunoresponsive cell, or the pharmaceutical composition of any one of the above aspects or embodiments to the subject in need thereof.

In another aspect, provided herein is a kit for treating and/or preventing a tumor, comprising the immunoresponsive cell or the pharmaceutical composition of any one of the the above aspects or embodiments, optionally wherein the kit further comprises written instructions for using the immunoresponsive cell or pharmaceutical composition for treating and/or preventing the tumor in a subject.

### BRIEF DESCRIPTION OF THE DRAWINGS

FIG. 1 depicts fold-change of geometric mean fluorescent intensity (gMFI) of mKate and YFP-expressing T cells driven by CCL3 promoter ablation variants.

FIG. 2A depicts mCherry (mKate) fluorescence expression, determined by flow cytometry, of stimulated and not stimulated NK cells at 24 hours. Fluorescence is driven by the CCL3 promoter ablation variants. FIG. 2B depicts mCherry fluorescence expression, determined by flow cytometry, of stimulated and not stimulated NK cells at 48 hours. Fluorescence is driven by the CCL3 promoter ablation variants.

FIG. 3A depicts gMFI of stimulated and not stimulated NK cells at 24 hours, with mCherry expression driven by the CCL3 promoter ablation variants. FIG. 3B depicts gMFI fold change following stimulation of the NK cells. FIG. 3C depicts stimulated plotted against not stimulated NK cell gMFI. FIG. 3D depicts stimulated plotted against not stimulated NK cell gMFI, with the 1682 outlier removed.

FIG. 4A depicts gMFI of stimulated and not stimulated NK cells at 48 hours, with mCherry expression driven by the CCL3 promoter ablation variants. FIG. 4B depicts gMFI fold change following stimulation of the NK cells. FIG. 4C depicts stimulated plotted against not stimulated NK cell gMFI. FIG. 4D depicts stimulated plotted against not stimulated NK cell gMFI, with the 1682 outlier removed.

FIG. 5A depicts gMFI of stimulated and unstimulated T cells, with BFP expression driven by various CCL3 promoter variants. FIG. 5B depicts the gMFI fold change following stimulation of the T cells.

FIG. 6A depicts gMFI of stimulated and unstimulated T cells, with BFP expression driven by various CCL3 promoter variants. FIG. 6B depicts the gMFI fold change following stimulation of the T cells.

### DETAILED DESCRIPTION

### Definitions

Terms used in the claims and specification are defined as set forth below unless otherwise specified.

The term “ameliorating” refers to any therapeutically beneficial result in the treatment of a disease state, e.g., a cancer disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-human animals, and includes but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

### Engineered Nucleic Acids and Polypeptides

Regulation of drug expression in cell therapies is required to hit therapeutic efficacy windows. Such methods are described herein using a regulatable transcription factor that can drive expression of any desirable effector molecule or combination of effector molecules. This system is versatile as it can regulate intracellular or membrane bound proteins by using, for example, a modular protease system that enables ON or OFF configurations. It can use protease switch drugs that are FDA approved and can be administered via oral delivery with a favorable pharmacokinetic profile. In addition, the methods and compositions described herein may be used, e.g., for regulated immunomodulatory effector expression in cell or gene therapies. The regulatable transcription factor can be used in conjunction with, e.g., CAR T cells, CAR NK cells, TCR T cells, TIL therapies, viral-specific T cells, or any other appropriate immune cell therapy.

### The CCL3 Inducible Promoter and Ablation Variants

CCL3 (also known in the art as C—C Motif Chemokine Ligand, MIP-1-Alpha, GOS19, LD78ALPHA, SCYA3, Small Inducible Cytokine A3 (Homologous To Mouse Mip-1a), Macrophage Inflammatory Protein 1-Alpha, Tonsillar Lymphocyte LD78 Alpha Protein, G0/G1 Switch Regulatory Protein 19-1, C—C Motif Chemokine 3, PAT 464.1, SIS-Beta, MIP1A, Chemokine (C—C Motif) Ligand 3, and Small-Inducible Cytokine A3) is a small, inducible cytokine. As the CCL3 promoter is inducible upon stimulation with an immune cell activation signal, it may be a tool for regulating expression of effector molecules. An “immune cell activation signal” as used herein refers engagement of a protein and/or protein complex sufficient for stimulating an immune response, as determined by activation of the CCL3 promoter, and includes, for example and without limitation, CD3 and 4-1BB, CD3 and CD28, etc. In certain embodiments, the immune cell activation signal comprises CD3 and CD28. In certain embodiments, the immune cell activation signal comprises CD3 and 4-1BB. In certain embodiments, the immune cell activation signal comprises a cell comprising the proteins, e.g., Huh7 cells.

The CCL3 promoter is more than about 2000 basepairs in length. In some embodiments, the CCL3 promoter comprises an ablation of a series of contiguous nucleotides within the promoter (referred to as a “nucleotide motif”). As used herein, an “ablation” can refer to a deletion using any means of nucleotide deletion as known in the art (e.g., molecular cloning, CRISPR, etc.), or can refer to a substitution (i.e., nucleotide substitution) such that at least one nucleotide is replaced with at least one other nucleotide. In some embodiments, ablation of the nucleotide motif increases inducibility of the CCL3 promoter (e.g., transcriptional levels downstream of the promoter following stimulation), wherein the increased inducibility is relative to the wildtype CCL3 promoter. Methods of quantifying transcriptional levels are known in the art and include, for example and without limitation, mRNA analysis by reverse-transcriptase quantitative polymerase chain reaction (RT-qPCR), fluorescent reporters, colorimetric reporters, etc. In some embodiments, the ablation increases inducibility by at least 0.5-fold, at least 1-fold, at least 2-fold, at least 3-fold, at least 4-fold, at least 5-fold, at least 6-fold, at least 7-fold, or at least 8-fold. It is contemplated herein that changes in inducibility of the CCL3 promoter may depend on the test system, e.g., the cell type comprising the promoter.

In some embodiments, the ablation comprises a substitution of a second nucleotide motif at the site of ablation. In some embodiments, introduction of the second nucleotide motif in the ablation site does not introduce new regulatory sites in the CCL3 promoter (e.g., transcription factor binding sites). Such CCL3 promoter comprising a deletion or a substitution of a nucleotide motif at the site of ablation are referred to herein as “ablation variants.”

Sequences of exemplary CCL3 promoter ablation variants are provided in Table 1 below. Sequences of the wildtype ablation motif and the second nucleotide motif used for substitution of the same ablation variants are provided in Table 2.

TABLE 1
DNA sequences of CCL3 promoter ablation variants

| | | SEQ |
|---|---|---|
| Promoter | | ID |
| ID | Sequence | NO |
| CCL3 | GGGGGAATACAAGGCCTGTCCTGGTTTGGTCCCAATTTAGCTT | 132 |
| wildtype | TATCATCCATATTCACCCCCACTGCTCTGCAGCTCCACTGAAG | |
| promoter | CACCCCCTCTTTCCTCTGAACCCACAATGTCACACTCAGGACT | |
| 2935 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 133 |
| ablation | TATCATCCATATTCACCTTACCTACTAGGTTAATCCACTGAAG | |
| variant | CACCCCCTCTTTCCTCTGAACCCACAATGTCACACTCAGGACT | |
| 4125 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 134 |
| ablation | TATCATCCATATTCACCCCCACTGCTCTGCAGCTCCACTGAAG | |
| variant | CACCCACTCGAATTCAGACGACAAAAATGTCACACTCAGGAC | |
| 4126 | TCTGCCTCAGCTGGGCACTCATCTATAGATGCCTAAATCCCGG | |
| | GCAGTTATCCAGACACAACTAAAGTTCCATCCCTTCCATGAAG | |
| | CCTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 135 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4127 | CTGCCTCAGCTGGGCACTCATCTATAGATGCCTAAATCCCGGG | |
| | CAGTTATCCAGACACAACTAAAGTTCCATTCTAAGTCCAATTC | |
| | ACGACAAGACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 136 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4128 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTTCAGACTCAATTATTCTTCCCCTCGTGGG | |
| | ATTCTGAGCTTTCATTTCTTTTTCTACTAGGAGTCCTAGCACTT | |
| | TCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 137 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4129 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGTACACGGTTAAGACTCAATACTAGGAGTCCTAGCAC | |
| | TTTCGGCTAAATGCTACAATTACCTGTTCATACACTCTACCTGC | |
| | CCCCACGAGATCAGGGGCATCTCAGAAACAAAGATCATTAAA | |
| | ACCAACTAAATCTATTTCTCATTATAAAATGAGGTATGCTGAT | |
| | TGATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 138 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4130 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGAAACGAGATTCTGTACGACTGTTCATACACTCTACCTGC | |
| | CCCCACGAGATCAGGGGCATCTCAGAAACAAAGATCATTAAA | |
| | ACCAACTAAATCTATTTCTCATTATAAAATGAGGTATGCTGAT | |
| | TGATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 139 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4131 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACATCTACACGCATTA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 140 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4132 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATATCTCGCTAATAGGAGTAAGATA | |
| | CAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 141 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4133 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGCAAGACCTATACTATCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 142 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4134 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGTGCTATAGTGCTGTATAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 143 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4135 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCGTACCAGAATAAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 144 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4136 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCTATATACCAGCG | |
| | AGTTCGATAAAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 145 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4137 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGACGAAGCAATACTTAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 146 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4138 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTCCCGATAGAAAGAATTGCTTAGGGTTGGCAAG | |
| | GAATATATATATATTTGTACAAATATATATGTGTATATGTACA | |
| | AATACATGTATATATAGTACAAATATATATATATATTTGTACA | |
| | ATTCTTCAGACTTTGTAGAATTTGTATAATGTCGTATCTTGCTT | |
| | TTTTTAACCACTGATGTTATAAGCATATTTATGCCACTTCATTC | |
| | ATTTTAGAGACTTAATAATAAATGATCTAGTGGATAATTTATC | |
| | ATTCCCTGATGGAGAAAAATTTAGCTTTGTTTATTTTAGAGTTA | |
| | TAAACGATGCTGGGTCAGGTATCTTTATGTTTGAAGATGGCTC | |
| | CATATTTGGGTTGTTTCCACAGAACTCTTTCCTAGAAATGCTTT | |
| | TTCTAGGTTAATGGCTACAGATATTTCTAGGCACCTGACATAT | |
| | TGACACCCACCTCTAAAGTATTTTTATGATCCACAACTAGCGT | |
| | TTAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 147 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4139 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTCTGTATAAAGCTCGTACCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 148 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4140 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACCATAGTGAGGAAATTCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 149 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4141 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGGTTAAGCATACTAAACGTTA | |
| | TAAACGATGCTGGGTCAGGTATCTTTATGTTTGAAGATGGCTC | |
| | CATATTTGGGTTGTTTCCACAGAACTCTTTCCTAGAAATGCTTT | |
| | TTCTAGGTTAATGGCTACAGATATTTCTAGGCACCTGACATAT | |
| | TGACACCCACCTCTAAAGTATTTTTATGATCCACAACTAGCGT | |
| | TTAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 150 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4142 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGAGTAAAGAAGCGAAATCTTTATGTTTGAAGATGGCTC | |
| | CATATTTGGGTTGTTTCCACAGAACTCTTTCCTAGAAATGCTTT | |
| | TTCTAGGTTAATGGCTACAGATATTTCTAGGCACCTGACATAT | |
| | TGACACCCACCTCTAAAGTATTTTTATGATCCACAACTAGCGT | |
| | TTAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 151 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4143 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGGTAAACCTCAA | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 152 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4144 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATAAAACACTAATTTCCACAGAACTCTTTCCTAGAAATGCTTT | |
| | TTCTAGGTTAATGGCTACAGATATTTCTAGGCACCTGACATAT | |
| | TGACACCCACCTCTAAAGTATTTTTATGATCCACAACTAGCGT | |
| | TTAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 153 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4145 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCGAGCGACGCTTAATATTGCTAGATATTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 154 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4146 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCAAACGCACCAGAACTATAACCACAACTAGCGT | |
| | TTAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 155 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4147 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTCAAGTCTGATAGTGAGTTGTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 156 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4148 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTCCGATCTCTAGTTAAGTTAGCTGTATAGCCCTC | |
| | CACAGCATCAGCCCATCAACCCTATCCCTGTGGTTATAGCAGC | |
| | TGAGGAAGCAGAATTGCAGCTCTGTGGGAAGGAATGGGGCTG | |
| | GAGAGTTCATGCACAGACCAGTTCTTATGAGAAGGGACTGAC | |
| | TAAGAATAGCCTTGGGTTGACATATACCCCTCTTCACACTCAC | |
| | AGGAGAAACCATTTCCCTATGAAACTATAACAAGTCATGAGTT | |
| | GAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 157 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4149 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCGTAGAACTCTTAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 158 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4150 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAATACCTTGGTGGAGCTCATCTAATATCTTCATATTACCTC | |
| | CAAGTTCATGCACAGACCAGTTCTTATGAGAAGGGACTGACTA | |
| | AGAATAGCCTTGGGTTGACATATACCCCTCTTCACACTCACAG | |
| | GAGAAACCATTTCCCTATGAAACTATAACAAGTCATGAGTTGA | |
| | GAGCTGAGAGTTAGAGAATAGCTCAAAGATGCTATTCTTGGAT | |
| | ATCCTGAGCCCCTGTGGTCACCAGGGACCCTGAGTTGTGCAAC | |
| | TTAGCATGACAGCATCACTACGCTTAAAAATTTCCCTCCTCAC | |
| | CCCCAGATTCCATTTCCCCATCCGCCAGGGCTGCCTATAAAGA | |
| | GGAGAGCTGGTTTCAGACTTCAGAAGGACACGGGCAGCAGAC | |
| | AGTGGTCAGTCCTTTCTTGGCTCTGCTGACACTCGAGCCCACA | |
| | TTCCGTCACCTGCTCAGAATC | |
| CCL3 | GGGGGAATACAAGGCCTGTCCTGGTTTGGTCCCAATTTAGCTT | 159 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4151 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CGATTGAACAAATTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 160 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4152 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCTATACCTTGGTTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 161 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4153 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGATTGCGTCAATTCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 162 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4154 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAAACCACGTATCT | |
| | ACTTATCCTGAGCCCCTGTGGTCACCAGGGACCCTGAGTTGTG | |
| | CAACTTAGCATGACAGCATCACTACGCTTAAAAATTTCCCTCC | |
| | TCACCCCCAGATTCCATTTCCCCATCCGCCAGGGCTGCCTATA | |
| | AAGAGGAGAGCTGGTTTCAGACTTCAGAAGGACACGGGCAGC | |
| | AGACAGTGGTCAGTCCTTTCTTGGCTCTGCTGACACTCGAGCC | |
| | CACATTCCGTCACCTGCTCAGAATC | |
| CCL3 | GGGGGAATACAAGGCCTGTCCTGGTTTGGTCCCAATTTAGCTT | 163 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4155 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCAAACTCTGCTACTCATCACTATAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 164 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4156 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGTGCTAGTT | |
| | GTCCAATAAAGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 165 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4157 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATCGACGTGTG | |
| | TCATATAGAATAACATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 166 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4158 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTTACAACCCTATAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 167 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4159 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTAGTCTAAGTTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 168 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 4160 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTGGAAGTAGCTTGTTTAATC | |
| CCL3 | CGTAGAACTTCGTAACATTAAGTCTCGTAGATAACGCCGTCAT | 169 |
| ablation | TGTATTTCTCTAACGACCCCACTGCTCTGCAGCTTTCTCAGCTA | |
| variant | ATATCCTCTTTCCTCTGAACCCACCAAGCTATGGCACTATGAT | |
| 4161 | AGTGACTATTATACTAACAGTACTGTAGGGCAATAGTCTGGTT | |
| | GTCGCTATTCCATATCGAGTGAATCTATCCCTTCCATGAAGCC | |
| | TTCCCCAACTTACAGGTGGAAGGTCACAACTAACCCATAACCG | |
| | TGTTAGAGCTTTCATTTCTTTTTCTAAGTTTGTGAGTGTACGAA | |
| | CATGCTAAATGCTACAATTACTTTATATTAGTTCATCACCATTT | |
| | GTGTCTTACTCTAACTTAGAGCGAAGTAAAGATCATTAAAATC | |
| | GTTCTGAATGGACTACACATATACAACTCGAAAGACTTCGCAC | |
| | TAAGCCGTCTGTATATTAACAAAGTATGGAAAAGAAAAAAAA | |
| | TTGACATGCTCTGTAGAAAAGGTAGAGACCCTTTTATCCGCTA | |
| | TCCAGATAGATACCAAAAGAATAAGTAAAAAATAGGAATGCT | |
| | ATAGCATGATTTATGTACATTCTCTTCGACGGACTATCATGCA | |
| | AGGTGTAAGGTAGCGATCTAAGACGTTTGTATCGAACTAAGTA | |
| | GACATTCTTGTGTATATGTTAGAGCATAATAATTTCTTTCAGAT | |
| | TAAACTTTATCCTTTGCGCACTTTGAGGGTCTATTTACACGATT | |
| | AATCTTGTCGACTGCTCATACAACAGCATAATGCGACTACGTC | |
| | GATTACAGAAGTCTTGGACATTACTTTCGCTTAAATATATCGA | |
| | AAAAGAAAAAAAACTAGCTTCGCGTAAAGCACTGACCAATTG | |
| | GTGTTGTATGTAATATCGGACCAAGATGACAAGTATACTCATA | |
| | ACGTATTGTATTTTTTTCCATGATTCGTACATGATGGTTATACA | |
| | GTACGTTTACTGTAGCACACTAAATAACTTAAGATCGAAACCG | |
| | AGTGATACGTACTTAAGTACGCAACGCTTACCGTCTACAGCAT | |
| | TGAGAATTATCCTACTGCATCTAAAGTCGCTATGCTACGACTG | |
| | GTAAGTTAAGTTATAAGCATATTTATGTTTCTAGAGTTGAATG | |
| | TCTCACTTAATAATAAATGAAGAGTTACATCGAAATCGGCATT | |
| | ATGATGTTCCACACATATCCTTTGTTTATTTTAGAAAATGACG | |
| | ATATGCTGGGTCAGGTTATGCGTTTCTTGAAGATGGCTCCAAT | |
| | TTTGGGTTGTTTCCACAGAACTCTTTCCTAGAAATGCTGTCGGT | |
| | ATCTATTACGAGAAGCCTGATCAATTTCGGCTTATTTAACCAA | |
| | TCCTCAATCTAAAGTATTTTTATGATGCCGTTCAGATCGATCTA | |
| | TAGACCATCCGAATTGAAGATACTGATGATAACTATCGGTAAA | |
| | TGAAACATGACCTCATGCTTTTCTCAAGCTGTTGAGATCGTGA | |
| | ACAAATTGCCTCTGGGAGGAGGAAGGGTTGTGCACTCTCGAA | |
| | TAGTGTCCTATGAGAGCTTTCAATCCTGTGGTTATCGAGATAT | |
| | AGGAAGCAGAATTGCAGCTCTGTGGGAAGGAATGGGGCTGGA | |
| | GTACAAGGACTCGGTAACTACGCAGAATATAGCTGTATACTA | |
| | AGAATAGCCTTTACAGACAGCTAAAGCCTCTTCACACTAGATA | |
| | TAGAAACCATTTCAGTTACATATAGAAGTATAAAGTAGCCCAA | |
| | TGAGCCCTTGATCGAACTACGTGAACAGATGCTATTCTTGGAG | |
| | AAGTTGAGAACCTGTGGTCACCAGGGACCCTGAGTTGTGCAA | |
| | CTTAGCATGACTTACGGAGTCATACGTTTGTTTTCCCTCCTCAC | |
| | CCCCAGATTCTTCGTCCCCATCCGCCTGTATTCCCTATAAAGA | |
| | GGAGAGTTACTCTTCTGATATCCCTTATCCGAATTCCAATGGG | |
| | ACTACCTTCCAACAATTGGCTCTGCATATGAGACCTACAAACC | |
| | ACCGTCACCTGCTCAGAATA | |
| CCL3 | GGGGGAATACAAGGCCTGTCCTGGTTTGGTCCCAATTTAGCTT | 1 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 5960 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAATTTCCACA | |
| | GAACTCTTTCCTAGAAATGCTTTTTCTAGGTTAATGGCTACAG | |
| | ATATTTCTAGGCACCTGACATATTGACACCCACCTCTAAAGTA | |
| | TTTTTATGATCCACAACTAGCGTTTAACACAGCGCCCTAGTCA | |
| | CTACATGACTAATAAATAGACAAATGACTGAAACATGACCTC | |
| | ATGCTTTCTATTCCTCCAGCTTTCATTCAGTTCTTTAGCCCTCC | |
| | ACAGCATCAGCCCATCAACCCTATCCCTGTGGTTATAGCAGCT | |
| | GAGTTCATGCACAGACCAGTTCTTATGAGAAGGGACTGACTA | |
| | AGAATAGCCTTGGGTTGACATATACCCCTCTTCACACTCACAG | |
| | GATTGCGTCAATTCCTATGAAACTATAACAAGTCATGAGTTGA | |
| | GAGCTGAGAGTTAGAGAATAGCTCAAAGATGCTATTCTTGGAT | |
| | ATCCTGAGCCCCTGTGGTCACCAGGGACCCTGAGTTGTGCAAC | |
| | TTAGCATGACAGCATCACTACGCTTAAAAATTTCCCTCCTCAC | |
| | CCCCAGATTCCATTTCCCCATCCGCCAGGGCTGCCTATAAAGA | |
| | GGAGAGCTGGTTTCAGACTTCAGAAGGACACGGGCAGCAGAC | |
| | AGTGGTCAGTCCTTTCTTGGCTCTGCTGACACTCGAGCCCACA | |
| | TTCCGTCACCTGCTCAGAATC | |
| CCL3 | GGGGGAATACAAGGCCTGTCCTGGTTTGGTCCCAATTTAGCTT | 2 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 5959 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAATTTCCACA | |
| | GAACTCTTTCCTAGAAATGCTTTTTCTAGGTTAATGGCTACAG | |
| | ATATTTCTAGGCACCTGACATATTGACACCCACCTCTAAAGTA | |
| | TTTTTATGATCCACAACTAGCGTTTAACACAGCGCCCTAGTCA | |
| | CTACATGACTAATAAATAGACAAATGACTGAAACATGACCTC | |
| | ATGCTTTCTATTCCTCCAGCTTTCATTCAGTTCTTTGCCTCTGG | |
| | GAGGAGGAAGGGTTGTGCAGCCCTCCACAGCATCAGCCCATC | |
| | AACCCTATCCCTGTGGTTATAGCAGCTGAGGAAGCAGAATTGC | |
| | AGCTCTGTGGGAAGGAATGGGGCTGGAGAGTTCATGCACAGA | |
| | CCAGTTCTTATGAGAAGGGACTGACTAAGAATAGCCTTGGGTT | |
| | GACATATACCCCTCTTCACACTCACAGGATTGCGTCAATTCCT | |
| | ATGAAACTATAACAAGTCATGAGTTGAGAGCTGAGAGTTAGA | |
| | GAATAGCTCAAAGATGCTATTCTTGGATATCCTGAGCCCCTGT | |
| | GGTCACCAGGGACCCTGAGTTGTGCAACTTAGCATGACAGCAT | |
| | CACTACGCTTAAAAATTTCCCTCCTCACCCCCAGATTCCATTTC | |
| | CCCATCCGCCAGGGCTGCCTATAAAGAGGAGAGCTGGTTTCA | |
| | GACTTCAGAAGGACACGGGCAGCAGACAGTGGTCAGTCCTTT | |
| | CTTGGCTCTGCTGACACTCGAGCCCACATTCCGTCACCTGCTC | |
| | AGAATC | |
| CCL3 | GGGGGAATACAAGGCCTGTCCTGGTTTGGTCCCAATTTAGCTT | 3 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 5958 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCAAGTATAGTATAGGATCCTTAAAATGGTTCAAAGA | |
| | AATGGGAAATCAAGACTTCATTTTGGCCAAAACCATTGAACA | |
| | GAAACTTCAGCATATTTATCAACTGACAACAACCTATTTTTCA | |
| | ACCAGTGATGTTGGAAATGTTTTTTTAAAAATTAGTTTATAAA | |
| | TTTGTGGGCTGACCAAGAAGGTAATAAAGTCTAACTAAGTAA | |
| | AATGAGAAAAATTCAGTAAGAAAATAAATCACCCAGGGACCT | |
| | ATCACACAAATATAAGAACTATTCATTCTTTAAGGCATGTATT | |
| | TCCAAGCCTTTGTATTTTTTTCCATGCTTAGGGTTGGCAAGGAA | |
| | TATATATATATTTGTACAAATATATATGTGTATATGTACAAAT | |
| | ACATGTATATATAGTACAAATATATATATATATTTGTACAATT | |
| | CTTCAGACTTTGTAGAATTTGTATAATGTCGTATCTTGCTTTTT | |
| | TTAACCACTGATGCCACTTCATTCATTTTAGAGATCTAGTGGA | |
| | TAATTTATCATTCCCTGATGGAGAAAAATTTAGGTTATAAACG | |
| | ATGCTGGGTCAGGTATCTTTATGTTTGAAGATGGCTCCATATTT | |
| | GGGTTGTTTCCACAGAACTCTTTCCTAGAAATGCTTTTTCTAGG | |
| | TTAATGGCTACAGATATTTCTAGGCACCTGACATATTGACACC | |
| | CACCTCTAAAGTATTTTTATGATCCACAACTAGCGTTTAACAC | |
| | AGCGCCCTAGTCACTACATGACTAATAAATAGACAAATGACT | |
| | GAAACATGACCTCATGCTTTCTATTCCTCCAGCTTTCATTCAGT | |
| | TCTTTAGCCCTCCACAGCATCAGCCCATCAACCCTATCCCTGT | |
| | GGTTATAGCAGCTGAGTTCATGCACAGACCAGTTCTTATGAGA | |
| | AGGGACTGACTAAGAATAGCCTTGGGTTGACATATACCCCTCT | |
| | TCACACTCACAGGCCTATGAAACTATAACAAGTCATGAGTTGA | |
| | GAGCTGAGAGTTAGAGAATAGCTCAAAGATGCTATTCTTGGAT | |
| | ATCCTGAGCCCCTGTGGTCACCAGGGACCCTGAGTTGTGCAAC | |
| | TTAGCATGACAGCATCACTACGCTTAAAAATTTCCCTCCTCAC | |
| | CCCCAGATTCCATTTCCCCATCCGCCAGGGCTGCCTATAAAGA | |
| | GGAGAGCTGGTTTCAGACTTCAGAAGGACACGGGCAGCAGAC | |
| | AGTGGTCAGTCCTTTCTTGGCTCTGCTGACACTCGAGCCCACA | |
| | TTCCGTCACCTGCTCAGAATC | |
| CCL3 | GGGGGAATACAAGGCCTGTCCTGGTTTGGTCCCAATTTAGCTT | 4 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 5957 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGATTGCGTCAATTCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 6 |
| wildtype | TGTTATTCTCCAGCATCAAATGTATCAACTCTAACCCCTTTGGG | |
| promoter | GGGAATACAAGGCCTGTCCTGGTTTGGTCCCAATTTAGCTTTA | |
| OH2935 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 7 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4125 | TCATCCATATTCACCTTACCTACTAGGTTAATCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 8 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4126 | TCATCCATATTCACCCCCACTGCTCTGCAGCTCCACTGAAGCA | |
| | CCCACTCGAATTCAGACGACAAAAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 9 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4127 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATTCTAAGTCCAATTCAC | |
| | GACAAGACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTICCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 10 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4128 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTTCAGACTCAATTATTCTTCCCCTCGTGGGAT | |
| | TCTGAGCTTTCATTTCTTTTTCTACTAGGAGTCCTAGCACTTTC | |
| | GGCTAAATGCTACAATTACCTGTTCATACACTCTACCTGCCCC | |
| | CACGAGATCAGGGGCATCTCAGAAACAAAGATCATTAAAACC | |
| | AACTAAATCTATTTCTCATTATAAAATGAGGTATGCTGATTGA | |
| | TTGTGAAAGAATAAAATAACAAAGTATGGAAAAGAAAAAAA | |
| | AGCATATAATCTGGCTGAGAAGGTAGAGACCCTTCCACACCA | |
| | CTGAAATTATGTATTGAAAAGAATAAGTAAAAAACTGCTTCA | |
| | ATTTGGCATGATTTATGTAAGTATAGTATAGGATCCTTAAAAT | |
| | GGTTCAAAGAAATGGGAAATCAAGACTTCATTTTGGCCAAAA | |
| | CCATTGAACAGAAACTTCAGCATATTTATCAATAATTTCTTTC | |
| | AGATTAAACAACTGACAACAACCTATTTTTCAACCAGTGATGT | |
| | TGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 11 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4129 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGTACACGGTTAAGACTCAATACTAGGAGTCCTAGCACTT | |
| | TCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 12 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4130 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGAAACGAGATTCTGTACGACTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 13 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4131 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACATCTACACGCATTAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 14 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4132 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATATCTCGCTAATAGGAGTAAGATAC | |
| | AGCATATAATCTGGCTGAGAAGGTAGAGACCCTTCCACACCA | |
| | CTGAAATTATGTATTGAAAAGAATAAGTAAAAAACTGCTTCA | |
| | ATTTGGCATGATTTATGTAAGTATAGTATAGGATCCTTAAAAT | |
| | GGTTCAAAGAAATGGGAAATCAAGACTTCATTTTGGCCAAAA | |
| | CCATTGAACAGAAACTTCAGCATATTTATCAATAATTTCTTTC | |
| | AGATTAAACAACTGACAACAACCTATTTTTCAACCAGTGATGT | |
| | TGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 15 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4133 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGCAAGACCTATACTATCCACACCA | |
| | CTGAAATTATGTATTGAAAAGAATAAGTAAAAAACTGCTTCA | |
| | ATTTGGCATGATTTATGTAAGTATAGTATAGGATCCTTAAAAT | |
| | GGTTCAAAGAAATGGGAAATCAAGACTTCATTTTGGCCAAAA | |
| | CCATTGAACAGAAACTTCAGCATATTTATCAATAATTTCTTTC | |
| | AGATTAAACAACTGACAACAACCTATTTTTCAACCAGTGATGT | |
| | TGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 16 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4134 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGTGCTATAGTGCTGTATAACTGCTTCA | |
| | ATTTGGCATGATTTATGTAAGTATAGTATAGGATCCTTAAAAT | |
| | GGTTCAAAGAAATGGGAAATCAAGACTTCATTTTGGCCAAAA | |
| | CCATTGAACAGAAACTTCAGCATATTTATCAATAATTTCTTTC | |
| | AGATTAAACAACTGACAACAACCTATTTTTCAACCAGTGATGT | |
| | TGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 17 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4135 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCGTACCAGAATAAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 18 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4136 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCTATATACCAGCG | |
| | AGTTCGATAAAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 19 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4137 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGACGAAGCAATACTTAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 20 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4138 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTCCCGATAGAAAGAATTGCTTAGGGTTGGCAAG | |
| | GAATATATATATATTTGTACAAATATATATGTGTATATGTACA | |
| | AATACATGTATATATAGTACAAATATATATATATATTTGTACA | |
| | ATTCTTCAGACTTTGTAGAATTTGTATAATGTCGTATCTTGCTT | |
| | TTTTTAACCACTGATGTTATAAGCATATTTATGCCACTTCATTC | |
| | ATTTTAGAGACTTAATAATAAATGATCTAGTGGATAATTTATC | |
| | ATTCCCTGATGGAGAAAAATTTAGCTTTGTTTATTTTAGAGTTA | |
| | TAAACGATGCTGGGTCAGGTATCTTTATGTTTGAAGATGGCTC | |
| | CATATTTGGGTTGTTTCCACAGAACTCTTTCCTAGAAATGCTTT | |
| | TTCTAGGTTAATGGCTACAGATATTTCTAGGCACCTGACATAT | |
| | TGACACCCACCTCTAAAGTATTTTTATGATCCACAACTAGCGT | |
| | TTAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 21 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4139 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTCTGTATAAAGCTCGTACCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 22 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4140 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACCATAGTGAGGAAATTCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 23 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4141 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGGTTAAGCATACTAAACGTTA | |
| | TAAACGATGCTGGGTCAGGTATCTTTATGTTTGAAGATGGCTC | |
| | CATATTTGGGTTGTTTCCACAGAACTCTTTCCTAGAAATGCTTT | |
| | TTCTAGGTTAATGGCTACAGATATTTCTAGGCACCTGACATAT | |
| | TGACACCCACCTCTAAAGTATTTTTATGATCCACAACTAGCGT | |
| | TTAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 24 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4142 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGAGTAAAGAAGCGAAATCTTTATGTTTGAAGATGGCTC | |
| | CATATTTGGGTTGTTTCCACAGAACTCTTTCCTAGAAATGCTTT | |
| | TTCTAGGTTAATGGCTACAGATATTTCTAGGCACCTGACATAT | |
| | TGACACCCACCTCTAAAGTATTTTTATGATCCACAACTAGCGT | |
| | TTAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 25 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4143 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGGTAAACCTCAA | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 26 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4144 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATAAAACACTAATTTCCACAGAACTCTTTCCTAGAAATGCTTT | |
| | TTCTAGGTTAATGGCTACAGATATTTCTAGGCACCTGACATAT | |
| | TGACACCCACCTCTAAAGTATTTTTATGATCCACAACTAGCGT | |
| | TTAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 27 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4145 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCGAGCGACGCTTAATATTGCTAGATATTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 28 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4146 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCAAACGCACCAGAACTATAACCACAACTAGCGT | |
| | TTAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 29 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4147 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTCAAGTCTGATAGTGAGTTGTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 30 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4148 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTCCGATCTCTAGTTAAGTTAGCTGTATAGCCCTC | |
| | CACAGCATCAGCCCATCAACCCTATCCCTGTGGTTATAGCAGC | |
| | TGAGGAAGCAGAATTGCAGCTCTGTGGGAAGGAATGGGGCTG | |
| | GAGAGTTCATGCACAGACCAGTTCTTATGAGAAGGGACTGAC | |
| | TAAGAATAGCCTTGGGTTGACATATACCCCTCTTCACACTCAC | |
| | AGGAGAAACCATTTCCCTATGAAACTATAACAAGTCATGAGTT | |
| | GAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 81 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4149 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCGTAGAACTCTTAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 82 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4150 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAATACCTTGGTGGAGCTCATCTAATATCTTCATATTACCTC | |
| | CAAGTTCATGCACAGACCAGTTCTTATGAGAAGGGACTGACTA | |
| | AGAATAGCCTTGGGTTGACATATACCCCTCTTCACACTCACAG | |
| | GAGAAACCATTTCCCTATGAAACTATAACAAGTCATGAGTTGA | |
| | GAGCTGAGAGTTAGAGAATAGCTCAAAGATGCTATTCTTGGAT | |
| | ATCCTGAGCCCCTGTGGTCACCAGGGACCCTGAGTTGTGCAAC | |
| | TTAGCATGACAGCATCACTACGCTTAAAAATTTCCCTCCTCAC | |
| | CCCCAGATTCCATTTCCCCATCCGCCAGGGCTGCCTATAAAGA | |
| | GGAGAGCTGGTTTCAGACTTCAGAAGGACACGGGCAGCAGAC | |
| | AGTGGTCAGTCCTTTCTTGGCTCTGCTGACACTCGAGCCCACA | |
| | TTCCGTCACCTGCTCAGAATC | |
| CCL3 | GGACAGAATTCCAAAGGCATGGTCGCACTTGGCTTCTGTCCTC | 88 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4151 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CGATTGAACAAATTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 89 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4152 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCTATACCTTGGTTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTIGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 90 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4153 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGATTGCGTCAATTCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 91 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4154 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAAACCACGTATCT | |
| | ACTTATCCTGAGCCCCTGTGGTCACCAGGGACCCTGAGTTGTG | |
| | CAACTTAGCATGACAGCATCACTACGCTTAAAAATTTCCCTCC | |
| | TCACCCCCAGATTCCATTTCCCCATCCGCCAGGGCTGCCTATA | |
| | AAGAGGAGAGCTGGTTTCAGACTTCAGAAGGACACGGGCAGC | |
| | AGACAGTGGTCAGTCCTTTCTTGGCTCTGCTGACACTCGAGCC | |
| | CACATTCCGTCACCTGCTCAGAATC | |
| CCL3 | GGACAGAATTCCAAAGGCATGGTCGCACTTGGCTTCTGTCCTC | 92 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4155 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCAAACTCTGCTACTCATCACTATAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 96 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4156 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGTGCTAGTT | |
| | GTCCAATAAAGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 97 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4157 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATCGACGTGTG | |
| | TCATATAGAATAACATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 119 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4158 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTTACAACCCTATAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGACAGAATTCCAAAGGCATGGTCGCACTTGGCTTCTGTCCTC | 120 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4159 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTAGTCTAAGTTGACACTCGAGCCC | |
| | ACATTCCGTCACCTGCTCAGAATC | |
| CCL3 | GGACAGAATTCCAAAGGCATGGTCGCACTTGGCTTCTGTCCTC | 121 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH4160 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGAGAAACCATTTCCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTGGAAGTAGCTTGTTTAATC | |
| CCL3 | GGACAGAATTCCAAAGGCATGGTCGCACTTGGCTTCTGTCCTC | 122 |
| ablation | TGTTATTCTCCAGCATCAAATGTATCAACTCTAACCCCTTTGCG | |
| variant | TAGAACTTCGTAACATTAAGTCTCGTAGATAACGCCGTCATTG | |
| OH4161 | TATTTCTCTAACGACCCCACTGCTCTGCAGCTTTCTCAGCTAAT | |
| | ATCCTCTTTCCTCTGAACCCACCAAGCTATGGCACTATGATAG | |
| | TGACTATTATACTAACAGTACTGTAGGGCAATAGTCTGGTTGT | |
| | CGCTATTCCATATCGAGTGAATCTATCCCTTCCATGAAGCCTTC | |
| | CCCAACTTACAGGTGGAAGGTCACAACTAACCCATAACCGTGT | |
| | TAGAGCTTTCATTTCTTTTTCTAAGTTTGTGAGTGTACGAACAT | |
| | GCTAAATGCTACAATTACTTTATATTAGTTCATCACCATTTGTG | |
| | TCTTACTCTAACTTAGAGCGAAGTAAAGATCATTAAAATCGTT | |
| | CTGAATGGACTACACATATACAACTCGAAAGACTTCGCACTAA | |
| | GCCGTCTGTATATTAACAAAGTATGGAAAAGAAAAAAAATTG | |
| | ACATGCTCTGTAGAAAAGGTAGAGACCCTTTTATCCGCTATCC | |
| | AGATAGATACCAAAAGAATAAGTAAAAAATAGGAATGCTATA | |
| | GCATGATTTATGTACATTCTCTTCGACGGACTATCATGCAAGG | |
| | TGTAAGGTAGCGATCTAAGACGTTTGTATCGAACTAAGTAGAC | |
| | ATTCTTGTGTATATGTTAGAGCATAATAATTTCTTTCAGATTAA | |
| | ACTTTATCCTTTGCGCACTTTGAGGGTCTATTTACACGATTAAT | |
| | CTTGTCGACTGCTCATACAACAGCATAATGCGACTACGTCGAT | |
| | TACAGAAGTCTTGGACATTACTTTCGCTTAAATATATCGAAAA | |
| | AGAAAAAAAACTAGCTTCGCGTAAAGCACTGACCAATTGGTG | |
| | TTGTATGTAATATCGGACCAAGATGACAAGTATACTCATAACG | |
| | TATTGTATTTTTTTCCATGATTCGTACATGATGGTTATACAGTA | |
| | CGTTTACTGTAGCACACTAAATAACTTAAGATCGAAACCGAGT | |
| | GATACGTACTTAAGTACGCAACGCTTACCGTCTACAGCATTGA | |
| | GAATTATCCTACTGCATCTAAAGTCGCTATGCTACGACTGGTA | |
| | AGTTAAGTTATAAGCATATTTATGTTTCTAGAGTTGAATGTCTC | |
| | ACTTAATAATAAATGAAGAGTTACATCGAAATCGGCATTATGA | |
| | TGTTCCACACATATCCTTTGTTTATTTTAGAAAATGACGATATG | |
| | CTGGGTCAGGTTATGCGTTTCTTGAAGATGGCTCCAATTTTGG | |
| | GTTGTTTCCACAGAACTCTTTCCTAGAAATGCTGTCGGTATCTA | |
| | TTACGAGAAGCCTGATCAATTTCGGCTTATTTAACCAATCCTC | |
| | AATCTAAAGTATTTTTATGATGCCGTTCAGATCGATCTATAGA | |
| | CCATCCGAATTGAAGATACTGATGATAACTATCGGTAAATGAA | |
| | ACATGACCTCATGCTTTTCTCAAGCTGTTGAGATCGTGAACAA | |
| | ATTGCCTCTGGGAGGAGGAAGGGTTGTGCACTCTCGAATAGTG | |
| | TCCTATGAGAGCTTTCAATCCTGTGGTTATCGAGATATAGGAA | |
| | GCAGAATTGCAGCTCTGTGGGAAGGAATGGGGCTGGAGTACA | |
| | AGGACTCGGTAACTACGCAGAATATAGCTGTATACTAAGAAT | |
| | AGCCTTTACAGACAGCTAAAGCCTCTTCACACTAGATATAGAA | |
| | ACCATTTCAGTTACATATAGAAGTATAAAGTAGCCCAATGAGC | |
| | CCTTGATCGAACTACGTGAACAGATGCTATTCTTGGAGAAGTT | |
| | GAGAACCTGTGGTCACCAGGGACCCTGAGTTGTGCAACTTAGC | |
| | ATGACTTACGGAGTCATACGTTTGTTTTCCCTCCTCACCCCCAG | |
| | ATTCTTCGTCCCCATCCGCCTGTATTCCCTATAAAGAGGAGAG | |
| | TTACTCTTCTGATATCCCTTATCCGAATTCCAATGGGACTACCT | |
| | TCCAACAATTGGCTCTGCATATGAGACCTACAAACCACCGTCA | |
| | CCTGCTCAGAATA | |
| CCL3 | GGACAGAATTCCAAAGGCATGGTCGCACTTGGCTTCTGTCCTC | 123 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH5960 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAATTTCCACA | |
| | GAACTCTTTCCTAGAAATGCTTTTTCTAGGTTAATGGCTACAG | |
| | ATATTTCTAGGCACCTGACATATTGACACCCACCTCTAAAGTA | |
| | TTTTTATGATCCACAACTAGCGTTTAACACAGCGCCCTAGTCA | |
| | CTACATGACTAATAAATAGACAAATGACTGAAACATGACCTC | |
| | ATGCTTTCTATTCCTCCAGCTTTCATTCAGTTCTTTAGCCCTCC | |
| | ACAGCATCAGCCCATCAACCCTATCCCTGTGGTTATAGCAGCT | |
| | GAGTTCATGCACAGACCAGTTCTTATGAGAAGGGACTGACTA | |
| | AGAATAGCCTTGGGTTGACATATACCCCTCTTCACACTCACAG | |
| | GATTGCGTCAATTCCTATGAAACTATAACAAGTCATGAGTTGA | |
| | GAGCTGAGAGTTAGAGAATAGCTCAAAGATGCTATTCTTGGAT | |
| | ATCCTGAGCCCCTGTGGTCACCAGGGACCCTGAGTTGTGCAAC | |
| | TTAGCATGACAGCATCACTACGCTTAAAAATTTCCCTCCTCAC | |
| | CCCCAGATTCCATTTCCCCATCCGCCAGGGCTGCCTATAAAGA | |
| | GGAGAGCTGGTTTCAGACTTCAGAAGGACACGGGCAGCAGAC | |
| | AGTGGTCAGTCCTTTCTTGGCTCTGCTGACACTCGAGCCCACA | |
| | TTCCGTCACCTGCTCAGAATC | |
| CCL3 | GGACAGAATTCCAAAGGCATGGTCGCACTTGGCTTCTGTCCTC | 124 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH5959 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAATTTCCACA | |
| | GAACTCTTTCCTAGAAATGCTTTTTCTAGGTTAATGGCTACAG | |
| | ATATTTCTAGGCACCTGACATATTGACACCCACCTCTAAAGTA | |
| | TTTTTATGATCCACAACTAGCGTTTAACACAGCGCCCTAGTCA | |
| | CTACATGACTAATAAATAGACAAATGACTGAAACATGACCTC | |
| | ATGCTTTCTATTCCTCCAGCTTTCATTCAGTTCTTTGCCTCTGG | |
| | GAGGAGGAAGGGTTGTGCAGCCCTCCACAGCATCAGCCCATC | |
| | AACCCTATCCCTGTGGTTATAGCAGCTGAGGAAGCAGAATTGC | |
| | AGCTCTGTGGGAAGGAATGGGGCTGGAGAGTTCATGCACAGA | |
| | CCAGTTCTTATGAGAAGGGACTGACTAAGAATAGCCTTGGGTT | |
| | GACATATACCCCTCTTCACACTCACAGGATTGCGTCAATTCCT | |
| | ATGAAACTATAACAAGTCATGAGTTGAGAGCTGAGAGTTAGA | |
| | GAATAGCTCAAAGATGCTATTCTTGGATATCCTGAGCCCCTGT | |
| | GGTCACCAGGGACCCTGAGTTGTGCAACTTAGCATGACAGCAT | |
| | CACTACGCTTAAAAATTTCCCTCCTCACCCCCAGATTCCATTTC | |
| | CCCATCCGCCAGGGCTGCCTATAAAGAGGAGAGCTGGTTTCA | |
| | GACTTCAGAAGGACACGGGCAGCAGACAGTGGTCAGTCCTTT | |
| | CTTGGCTCTGCTGACACTCGAGCCCACATTCCGTCACCTGCTC | |
| | AGAATC | |
| CCL3 | GGACAGAATTCCAAAGGCATGGTCGCACTTGGCTTCTGTCCTC | 125 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH5958 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCAAGTATAGTATAGGATCCTTAAAATGGTTCAAAGA | |
| | AATGGGAAATCAAGACTTCATTTTGGCCAAAACCATTGAACA | |
| | GAAACTTCAGCATATTTATCAACTGACAACAACCTATTTTTCA | |
| | ACCAGTGATGTTGGAAATGTTTTTTTAAAAATTAGTTTATAAA | |
| | TTTGTGGGCTGACCAAGAAGGTAATAAAGTCTAACTAAGTAA | |
| | AATGAGAAAAATTCAGTAAGAAAATAAATCACCCAGGGACCT | |
| | ATCACACAAATATAAGAACTATTCATTCTTTAAGGCATGTATT | |
| | TCCAAGCCTTTGTATTTTTTTCCATGCTTAGGGTTGGCAAGGAA | |
| | TATATATATATTTGTACAAATATATATGTGTATATGTACAAAT | |
| | ACATGTATATATAGTACAAATATATATATATATTTGTACAATT | |
| | CTTCAGACTTTGTAGAATTTGTATAATGTCGTATCTTGCTTTTT | |
| | TTAACCACTGATGCCACTTCATTCATTTTAGAGATCTAGTGGA | |
| | TAATTTATCATTCCCTGATGGAGAAAAATTTAGGTTATAAACG | |
| | ATGCTGGGTCAGGTATCTTTATGTTTGAAGATGGCTCCATATTT | |
| | GGGTTGTTTCCACAGAACTCTTTCCTAGAAATGCTTTTTCTAGG | |
| | TTAATGGCTACAGATATTTCTAGGCACCTGACATATTGACACC | |
| | CACCTCTAAAGTATTTTTATGATCCACAACTAGCGTTTAACAC | |
| | AGCGCCCTAGTCACTACATGACTAATAAATAGACAAATGACT | |
| | GAAACATGACCTCATGCTTTCTATTCCTCCAGCTTTCATTCAGT | |
| | TCTTTAGCCCTCCACAGCATCAGCCCATCAACCCTATCCCTGT | |
| | GGTTATAGCAGCTGAGTTCATGCACAGACCAGTTCTTATGAGA | |
| | AGGGACTGACTAAGAATAGCCTTGGGTTGACATATACCCCTCT | |
| | TCACACTCACAGGCCTATGAAACTATAACAAGTCATGAGTTGA | |
| | GAGCTGAGAGTTAGAGAATAGCTCAAAGATGCTATTCTTGGAT | |
| | ATCCTGAGCCCCTGTGGTCACCAGGGACCCTGAGTTGTGCAAC | |
| | TTAGCATGACAGCATCACTACGCTTAAAAATTTCCCTCCTCAC | |
| | CCCCAGATTCCATTTCCCCATCCGCCAGGGCTGCCTATAAAGA | |
| | GGAGAGCTGGTTTCAGACTTCAGAAGGACACGGGCAGCAGAC | |
| | AGTGGTCAGTCCTTTCTTGGCTCTGCTGACACTCGAGCCCACA | |
| | TTCCGTCACCTGCTCAGAATC | |
| CCL3 | GGACAGAATTCCAAAGGCATGGTCGCACTTGGCTTCTGTCCTC | 126 |
| ablation | TGTTATTCTCCAGCATCAAATGTATCAACTCTAACCCCTTTGGG | |
| variant | GGGAATACAAGGCCTGTCCTGGTTTGGTCCCAATTTAGCTTTA | |
| OH5957 | TCATCCATATTCACCCCCACTGCTCTGCAGCTCCACTGAAGCA | |
| | CCCCCTCTTTCCTCTGAACCCACAATGTCACACTCAGGACTCT | |
| | GCCTCAGCTGGGCACTCATCTATAGATGCCTAAATCCCGGGCA | |
| | GTTATCCAGACACAACTAAAGTTCCATCCCTTCCATGAAGCCT | |
| | TCCCCAACCCTCTGGTGGAAGGTCACTTCTTCCCCTCGTGGGA | |
| | TTCTGAGCTTTCATTTCTTTTTCTACTAGGAGTCCTAGCACTTT | |
| | CGGCTAAATGCTACAATTACCTGTTCATACACTCTACCTGCCC | |
| | CCACGAGATCAGGGGCATCTCAGAAACAAAGATCATTAAAAC | |
| | CAACTAAATCTATTTCTCATTATAAAATGAGGTATGCTGATTG | |
| | ATTGTGAAAGAATAAAATAACAAAGTATGGAAAAGAAAAAA | |
| | AAGCATATAATCTGGCTGAGAAGGTAGAGACCCTTCCACACC | |
| | ACTGAAATTATGTATTGAAAAGAATAAGTAAAAAACTGCTTC | |
| | AATTTGGCATGATTTATGTAAGTATAGTATAGGATCCTTAAAA | |
| | TGGTTCAAAGAAATGGGAAATCAAGACTTCATTTTGGCCAAA | |
| | ACCATTGAACAGAAACTTCAGCATATTTATCAATAATTTCTTT | |
| | CAGATTAAACAACTGACAACAACCTATTTTTCAACCAGTGATG | |
| | TTGGAAATGTTTTTTTAAAAATTAGTTTATAAATTTGTGGGCTG | |
| | ACCAAGAAGGTAATAAAGTCTAACTAAGTAAAATGAGAAAAA | |
| | TTCAGAAAAAGAAAAAAATAAGAAAATAAATCACCCAGGGAC | |
| | CTATCACACAAATATAAGAACTATTCATTCTTTAAGGCATGTA | |
| | TTTCCAAGCCTTTGTATTTTTTTCCATGCTTAGGGTTGGCAAGG | |
| | AATATATATATATTTGTACAAATATATATGTGTATATGTACAA | |
| | ATACATGTATATATAGTACAAATATATATATATATTTGTACAA | |
| | TTCTTCAGACTTTGTAGAATTTGTATAATGTCGTATCTTGCTTT | |
| | TTTTAACCACTGATGTTATAAGCATATTTATGCCACTTCATTCA | |
| | TTTTAGAGACTTAATAATAAATGATCTAGTGGATAATTTATCA | |
| | TTCCCTGATGGAGAAAAATTTAGCTTTGTTTATTTTAGAGTTAT | |
| | AAACGATGCTGGGTCAGGTATCTTTATGTTTGAAGATGGCTCC | |
| | ATATTTGGGTTGTTTCCACAGAACTCTTTCCTAGAAATGCTTTT | |
| | TCTAGGTTAATGGCTACAGATATTTCTAGGCACCTGACATATT | |
| | GACACCCACCTCTAAAGTATTTTTATGATCCACAACTAGCGTT | |
| | TAACACAGCGCCCTAGTCACTACATGACTAATAAATAGACAA | |
| | ATGACTGAAACATGACCTCATGCTTTCTATTCCTCCAGCTTTCA | |
| | TTCAGTTCTTTGCCTCTGGGAGGAGGAAGGGTTGTGCAGCCCT | |
| | CCACAGCATCAGCCCATCAACCCTATCCCTGTGGTTATAGCAG | |
| | CTGAGGAAGCAGAATTGCAGCTCTGTGGGAAGGAATGGGGCT | |
| | GGAGAGTTCATGCACAGACCAGTTCTTATGAGAAGGGACTGA | |
| | CTAAGAATAGCCTTGGGTTGACATATACCCCTCTTCACACTCA | |
| | CAGGATTGCGTCAATTCCTATGAAACTATAACAAGTCATGAGT | |
| | TGAGAGCTGAGAGTTAGAGAATAGCTCAAAGATGCTATTCTTG | |
| | GATATCCTGAGCCCCTGTGGTCACCAGGGACCCTGAGTTGTGC | |
| | AACTTAGCATGACAGCATCACTACGCTTAAAAATTTCCCTCCT | |
| | CACCCCCAGATTCCATTTCCCCATCCGCCAGGGCTGCCTATAA | |
| | AGAGGAGAGCTGGTTTCAGACTTCAGAAGGACACGGGCAGCA | |
| | GACAGTGGTCAGTCCTTTCTTGGCTCTGCTGACACTCGAGCCC | |
| | ACATTCCGTCACCTGCTCAGAATC | |
| CCL3 | GGGGGAATACAAGGCCTGTCCTGGTTTGGTCCCAATTTAGCTT | 242 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 7293 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAATTTCCACA | |
| | GAACTCTTTCCTAGAAATGCTTTTTCTAGGTTAATGGCTACAG | |
| | ATATTTCTAGGCACCTGACATATTGACACCCACCTCTAAAGTA | |
| | TTTTTATGATCCACAACTAGCGTTTAACACAGCGCCCTAGTCA | |
| | CTACATGACTAATAAATAGACAAATGACCTATTCCTCCAGCTT | |
| | TCATTCAGTTCTTTAGCCCTCCACAGCATCAGCCCATCAACCCT | |
| | ATCCCTGTGGTTATAGCAGCTGAGTTCATGCACAGACCAGTTC | |
| | TTATGAGAAGGGACTGACTAAGAATAGCCTTGGGTTGACATAT | |
| | ACCCACAGGATTGCGTCAATTCCTATGAAACTATAACAAGTCA | |
| | TGAGTTGAGAGCTGAGAGTTAGAGAATAGCTCAAAGATGCTA | |
| | TTCTTGGATATCCTGAGCCCCTGTGGTCACCAGGGACCCTGAG | |
| | TTGTGCAACTTAGCATGACAGCATCACTACGCTTAAAAATTTC | |
| | CCTCCTCACCCCCAGATTCCATTTCCCCATCCGCCAGGGCTGC | |
| | CTATAAAGAGGAGAGCTGGTTTCAGACTTCAGAAGGACACGG | |
| | GCAGCAGACAGTGGTCAGTCCTTTCTTGGCTCTGCTGACACTC | |
| | GAGCCCACATTCCGTCACCTGCTCAGAATC | |
| CCL3 | GGGGGAATACAAGGCCTGTCCTGGTTTGGTCCCAATTTAGCTT | 243 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 7294 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAATTTCCACA | |
| | GAACTCTTTCCTAGAAATGCTTTTTCTAGGTTAATGGCTACAG | |
| | ATATTTCTAGGCACCTGACATATTGACACCCACCTCTAAAGTA | |
| | TTTTTATGATCCACAACTAGCGTTTAACACAGCGCCCTAGTCA | |
| | CTACATGACTAATAAATAGACAAATGACTGAAACATGACCTC | |
| | ATGCTTTCTATTCCTCCAGCTTTCATTCAGTTCTTTGCCTCTGG | |
| | GAGGAGGAAGGGTTGTGCAGCCCTCCACAGCATCAGCCCATC | |
| | AACCCTATCCCTGTGGTTATAGCAGCTGAGTTCATGCACAGAC | |
| | CAGTTCTTATGAGAAGGGACTGACTAAGAATAGCCTTGGGTTG | |
| | ACATATACCCACAGGATTGCGTCAATTCCTATGAAACTATAAC | |
| | AAGTCATGAGTTGAGAGCTGAGAGTTAGAGAATAGCTCAAAA | |
| | GCATCACTACGCTTAAAAATTTCCCTCCTCACCCCCAGATTCC | |
| | ATTTCCCCATCCGCCAGGGCTGCCTATAAAGAGGAGAGCTGGT | |
| | TTCAGACTTCAGAAGGACACGGGCAGCAGACAGTGGTCAGTC | |
| | CTTTCTTGGCTCTGCTGACACTCGAGCCCACATTCCGTCACCTG | |
| | CTCAGAATC | |
| CCL3 | GGGGGAATACAAGGCCTGTCCTGGTTTGGTCCCAATTTAGCTT | 244 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 7295 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAATTTCCACA | |
| | GAACTCTTTCCTAGAAATGCTTTTTCTAGGTTAATGGCTACAG | |
| | ATATTTCTAGGCACCTGACATATTGACACCCACCTCTAAAGTA | |
| | TTTTTATGATCCACAACTAGCGTTTAACACAGCGCCCTAGTCA | |
| | CTACATGACTAATAAATAGACAAATGACAGCCCTCCACAGCA | |
| | TCAGCCCATCAACCCTATCCCTGTGGTTATAGCAGCTGAGTTC | |
| | ATGCACAGACCAGTTCTTATGAGAAGGGACTGACTAAGAATA | |
| | GCCTTGGGTTGACATATACCCACAGGATTGCGTCAATTCCTAT | |
| | GAAACTATAACAAGTCATGAGTTGAGAGCTGAGAGTTAGAGA | |
| | ATAGCTCAAAAGCATCACTACGCTTAAAAATTTCCCTCCTCAC | |
| | CCCCAGATTCCATTT | |
| CCL3 | GGGGGAATACAAGGCCTGTCCTGGTTTGGTCCCAATTTAGCTT | 245 |
| ablation | TATCATCCATATTCACCCCCACTGCTCTGCAGCTCCACTGAAG | |
| variant | CACCCCCTCTTTCCTCTGAACCCACAATGTCACACTCAGGACT | |
| 7296 | CTGCCTCAGCTGGGCACTCATCTATAGATGCCTAAATCCCGGG | |
| | CAGTTATCCAGACACAACTAAAGTTCCATCCCTTCCATGAAGC | |
| | CTTCCCCAACCCTCTGGTGGAAGGTCACTTCTTCCCCTCGTGG | |
| | GATTCTGAGCTTTCATTTCTTTTTCTACTAGGAGTCCTAGCACT | |
| | TTCGGCTAAATGCTACAATTACCTGTTCATACACTCTACCTGCC | |
| | CCCACGAGATCAGGGGCATCTCAGAAACAAAGATCATTAAAA | |
| | CCAACTAAATCTATTTCTCATTATAAAATGAGGTATGCTGATT | |
| | GATTGTGAAAGAATAAAATAACAAAGTATGGAAAAGAAAAA | |
| | AAAGCATATAATCTGGCTGAGAAGGTAGAGACCCTTCCACAC | |
| | CACTGAAATTATGTATTGAAAAGAATAAGTAAAAATTTCCACA | |
| | GAACTCTTTCCTAGAAATGCTTTTTCTAGGTTAATGGCTACAG | |
| | ATATTTCTAGGCACCTGACATATTGACACCCACCTCTAAAGTA | |
| | TTTTTATGATCCACAACTAGCGTTTAACACAGCGCCCTAGTCA | |
| | CTACATGACTAATAAATAGACAAATGACAGCCCTCCACAGCA | |
| | TCAGCCCATCAACCCTATCCCTGTGGTTATAGCAGCTGAGTTC | |
| | ATGCACAGACCAGTTCTTATGAGAAGGGACTGACTAAGAATA | |
| | GCCTTGGGTTGACATATACCAGCATCACTACGCTTAAAAATTT | |
| | CCCTCCTCACCCCCAGATTCCATTT | |
| CCL3 | CCCCACTGCTCTGCAGCTCCTCTTTCCTCTGAACCCACTCCCTT | 246 |
| ablation | CCATGAAGCCTTCCCCAAGGTGGAAGGTCACGAGCTTTCATTT | |
| variant | CTTTTTCTGCTAAATGCTACAATTACAAGATCATTAAAAACAA | |
| 7297 | AGTATGGAAAAGAAAAAAAAAAGGTAGAGACCCTAAAAGAA | |
| | TAAGTAAAAATTTCCACAGAACTCTTTCCTAGAAATGCTTCTA | |
| | AAGTATTTTTATGATCCTGTGGTTATCTAAGAATAGCCTTCCCT | |
| | CCTCACCCCCAGATTC | |

TABLE 2
DNA sequences of the wildtype ablation motifs and the second nucleotide
motifs for CCL3 ablation variants with substitutions described in Table 1
above.

| Promoter | | |
|---|---|---|
| ID | Wildtype Sequence | Second Nucleotide Motif |
| CCL3 | CCCCACTGCTCTGCAGCT | CTTACCTACTAGGTTAAT |
| ablation | SEQ ID NO: 247 | SEQ ID NO: 171 |
| variant | Reverse-complement | |
| 4125 | AGCTGCAGAGCAGTGGGG | |
| | SEQ ID NO: 170 | |
| CCL3 | CCTCTTTCCTCTGAACCCAC | ACTCGAATTCAGACGACAAA |
| ablation | SEQ ID NO: 172 | SEQ ID NO: 173 |
| variant | Reverse-complement | |
| 4126 | GTGGGTTCAGAGGAAAGAGG | |
| | SEQ ID NO: 248 | |
| CCL3 | TCCCTTCCATGAAGCCTTCCCCA | TTCTAAGTCCAATTCACGACAAG |
| ablation | A | A |
| variant | SEQ ID NO: 249 | SEQ ID NO: 175 |
| 4127 | Reverse-complement | |
| | TTGGGGAAGGCTTCATGGAAGG | |
| | GA | |
| | SEQ ID NO: 174 | |
| CCL3 | GGTGGAAGGTCAC | TCAGACTCAATTA |
| ablation | SEQ ID NO: 176 | SEQ ID NO: 177 |
| variant | Reverse-complement | |
| 4128 | GTGACCTTCCACC | |
| | SEQ ID NO: 250 | |
| CCL3 | GAGCTTTCATTTCTTTTTCT | GTACACGGTTAAGACTCAAT |
| ablation | SEQ ID NO: 178 | SEQ ID NO: 179 |
| variant | Reverse-complement | |
| 4129 | AGAAAAAGAAATGAAAGCTC | |
| | SEQ ID NO: 251 | |
| CCL3 | GCTAAATGCTACAATTAC | AAACGAGATTCTGTACGA |
| ablation | SEQ ID NO: 180 | SEQ ID NO: 181 |
| variant | Reverse-complement | |
| 4130 | GTAATTGTAGCATTTAGC | |
| | SEQ ID NO: 252 | |
| CCL3 | AAGATCATTAAAA | TCTACACGCATTA |
| ablation | SEQ ID NO: 253 | SEQ ID NO: 183 |
| variant | Reverse-complement | |
| 4131 | TTTTAATGATCTT | |
| | SEQ ID NO: 182 | |
| CCL3 | ACAAAGTATGGAAAAGAAAAA | TCTCGCTAATAGGAGTAAGATAC |
| ablation | AAA | A |
| variant | SEQ ID NO: 184 | SEQ ID NO: 185 |
| 4132 | Reverse-complement | |
| | TTTTTTTTCTTTTCCATACTTTGT | |
| | SEQ ID NO: 254 | |
| CCL3 | AAGGTAGAGACCCT | CAAGACCTATACTA |
| ablation | SEQ ID NO: 186 | SEQ ID NO: 187 |
| variant | Reverse-complement | |
| 4133 | AGGGTCTCTACCTT | |
| | SEQ ID NO: 255 | |
| CCL3 | AAAAGAATAAGTAAAAA | TGCTATAGTGCTGTATA |
| ablation | SEQ ID NO: 256 | SEQ ID NO: 189 |
| variant | Reverse-complement | |
| 4134 | TTTTTACTTATTCTTTT | |
| | SEQ ID NO: 188 | |
| CCL3 | ATGATTTATGT | GTACCAGAATA |
| ablation | SEQ ID NO: 190 | SEQ ID NO: 191 |
| variant | Reverse-complement | |
| 4135 | ACATAAATCAT | |
| | SEQ ID NO: 257 | |
| CCL3 | AATAATTTCTTTCAGATTAAAC | TATATACCAGCGAGTTCGATAA |
| ablation | SEQ ID NO: 258 | SEQ ID NO: 193 |
| variant | Reverse-complement | |
| 4136 | GTTTAATCTGAAAGAAATTATT | |
| | SEQ ID NO: 192 | |
| CCL3 | AAAAAGAAAAAAA | ACGAAGCAATACT |
| ablation | SEQ ID NO: 194 | SEQ ID NO: 195 |
| variant | Reverse-complement | |
| 4137 | TTTTTTTCTTTTT | |
| | SEQ ID NO: 259 | |
| CCL3 | TTGTATTTTTTTCCAT | CCCGATAGAAAGAATT |
| ablation | SEQ ID NO: 196 | SEQ ID NO: 197 |
| variant | Reverse-complement | |
| 4138 | ATGGAAAAAAATACAA | |
| | SEQ ID NO: 260 | |
| CCL3 | TTATAAGCATATTTATG | TCTGTATAAAGCTCGTA |
| ablation | SEQ ID NO: 198 | SEQ ID NO: 199 |
| variant | Reverse-complement | |
| 4139 | CATAAATATGCTTATAA | |
| | SEQ ID NO: 261 | |
| CCL3 | CTTAATAATAAATGA | CCATAGTGAGGAAAT |
| ablation | SEQ ID NO: 262 | SEQ ID NO: 201 |
| variant | Reverse Complement | |
| 4140 | TCATTTATTATTAAG | |
| | SEQ ID NO: 200 | |
| CCL3 | CTTTGTTTATTTTAGA | GTTAAGCATACTAAAC |
| ablation | SEQ ID NO: 202 | SEQ ID NO: 203 |
| variant | Reverse Complement | |
| 4141 | TCTAAAATAAACAAAG | |
| | SEQ ID NO: 263 | |
| CCL3 | TGCTGGGTCAGGT | GTAAAGAAGCGAA |
| ablation | SEQ ID NO: 204 | SEQ ID NO: 205 |
| variant | Reverse-complement: | |
| 4142 | ACCTGACCCAGCA | |
| | SEQ ID NO: 264 | |
| CCL3 | GAAGATGGCTCCA | GGTAAACCTCAAA |
| ablation | SEQ ID NO: 206 | SEQ ID NO: 207 |
| variant | Reverse-complement: | |
| 4143 | TGGAGCCATCTTC | |
| | SEQ ID NO: 265 | |
| CCL3 | TTTGGGTTG | AAACACTAA |
| ablation | SEQ ID NO: 208 | SEQ ID NO: 209 |
| variant | Reverse-complement: | |
| 4144 | CAACCCAAA | |
| | SEQ ID NO: 266 | |
| CCL3 | TTTCCACAGAACTCTTTCCTAGA | TTTCGAGCGACGCTTAATATTGCT |
| ablation | AATGCT | AGATA |
| variant | SEQ ID NO: 210 | SEQ ID NO: 211 |
| 4145 | Reverse-complement: | |
| | AGCATTTCTAGGAAAGAGTTCT | |
| | GTGGAAA | |
| | SEQ ID NO: 267 | |
| CCL3 | TCTAAAGTATTTTTATGAT | AAACGCACCAGAACTATAA |
| ablation | SEQ ID NO: 212 | SEQ ID NO: 213 |
| variant | Reverse-complement: | |
| 4146 | ATCATAAAAATACTTTAGA | |
| | SEQ ID NO: 268 | |
| CCL3 | TGAAACATGACCTCATGCTTT | TCAAGTCTGATAGTGAGTTGT |
| ablation | SEQ ID NO: 214 | SEQ ID NO: 215 |
| variant | Reverse-complement: | |
| 4147 | AAAGCATGAGGTCATGTTTCA | |
| | SEQ ID NO: 269 | |
| CCL3 | GCCTCTGGGAGGAGGAAGGGTT | CCGATCTCTAGTTAAGTTAGCTGT |
| ablation | GTGC | AT |
| variant | SEQ ID NO: 216 | SEQ ID NO: 217 |
| 4148 | Reverse-complement: | |
| | GCACAACCCTTCCTCCTCCCAGA | |
| | GGC | |
| | SEQ ID NO: 270 | |
| CCL3 | CCTGTGGTTAT | GTAGAACTCTT |
| ablation | SEQ ID NO: 218 | SEQ ID NO: 219 |
| variant | Reverse-complement: | |
| 4149 | ATAACCACAGG | |
| | SEQ ID NO: 271 | |
| CCL3 | AGGAAGCAGAATTGCAGCTCTG | AATACCTTGGTGGAGCTCATCTAA |
| ablation | TGGGAAGGAATGGGGCTGGAG | TATCTTCATATTACCTCCA |
| variant | SEQ ID NO: 220 | SEQ ID NO: 221 |
| 4150 | Reverse-complement: | |
| | CTCCAGCCCCATTCCTTCCCACA | |
| | GAGCTGCAATTCTGCTTCCT | |
| | SEQ ID NO: 272 | |
| CCL3 | CTAAGAATAGCC | CGATTGAACAAA |
| ablation | SEQ ID NO: 273 | SEQ ID NO: 223 |
| variant | Reverse complement: | |
| 4151 | GGCTATTCTTAG | |
| | SEQ ID NO: 222 | |
| CCL3 | CCTCTTCACACT | TATACCTTGGTT |
| ablation | SEQ ID NO: 274 | SEQ ID NO: 225 |
| variant | Reverse complement: | |
| 4152 | AGTGTGAAGAGG | |
| | SEQ ID NO: 224 | |
| CCL3 | AGAAACCATTTC | ATTGCGTCAATT |
| ablation | SEQ ID NO: 275 | SEQ ID NO: 227 |
| variant | Reverse-complement: | |
| 4153 | GAAATGGTTTCT | |
| | SEQ ID NO: 226 | |
| CCL3 | GATGCTATTCTTGGA | AACCACGTATCTACT |
| ablation | SEQ ID NO: 228 | SEQ ID NO: 229 |
| variant | Reverse complement: | |
| 4154 | TCCAAGAATAGCATC | |
| | SEQ ID NO: 276 | |
| CCL3 | CCTGTGGTCACCAGGGACCCTG | AAACTCTGCTACTCATCACTAT |
| ablation | SEQ ID NO: 230 | SEQ ID NO: 231 |
| variant | Reverse complement: | |
| 4155 | CAGGGTCCCTGGTGACCACAGG | |
| | SEQ ID NO: 277 | |
| CCL3 | AGTTGTGCAACTTAGCATGAC | TGCTAGTTGTCCAATAAAGAC |
| ablation | SEQ ID NO: 232 | SEQ ID NO: 233 |
| variant | Reverse-complement: | |
| 4156 | GTCATGCTAAGTTGCACAACT | |
| | SEQ ID NO: 278 | |
| CCL3 | TTCCCTCCTCACCCCCAGATTC | CGACGTGTGTCATATAGAATAA |
| ablation | SEQ ID NO: 279 | SEQ ID NO: 235 |
| variant | Reverse-complement: | |
| 4157 | GAATCTGGGGGTGAGGAGGGAA | |
| | SEQ ID NO: 234 | |
| CCL3 | CCCCATCCGCC | TACAACCCTAT |
| ablation | SEQ ID NO: 280 | SEQ ID NO: 237 |
| variant | Reverse-complement: | |
| 4158 | GGCGGATGGGG | |
| | SEQ ID NO: 236 | |
| CCL3 | TGGCTCTGC | AGTCTAAGT |
| ablation | SEQ ID NO: 281 | SEQ ID NO: 239 |
| variant | Reverse-complement: | |
| 4159 | GCAGAGCCA | |
| | SEQ ID NO: 238 | |
| CCL3 | CCGTCACCTGCTCAGAA | GGAAGTAGCTTGTTTAA |
| ablation | SEQ ID NO: 282 | SEQ ID NO: 241 |
| variant | Revesre-complement: | |
| 4160 | TTCTGAGCAGGTGACGG | |
| | SEQ ID NO: 240 | |

In some embodiments, a CCL3 promoter of the present disclosure may comprise an ablation of at least two nucleotide motifs relative to SEQ ID NO:132. In some embodiments, a CCL3 promoter of the present disclosure may comprise an ablation of at least three nucleotide motifs relative to SEQ ID NO:132. In some embodiments, a CCL3 promoter of the present disclosure may comprise an ablation of at least four nucleotide motifs relative to SEQ ID NO:132. In some embodiments, a CCL3 promoter of the present disclosure may comprise an ablation of at least five nucleotide motifs relative to SEQ ID NO:132. In some embodiments, a CCL3 promoter of the present disclosure may comprise an ablation of at least six nucleotide motifs relative to SEQ ID NO:132. In some embodiments, a CCL3 promoter of the present disclosure may comprise an ablation of at least seven nucleotide motif relative to SEQ ID NO:132s. In some embodiments, a CCL3 promoter of the present disclosure may comprise an ablation of at least eight nucleotide motifs relative to SEQ ID NO:132. In some embodiments, a CCL3 promoter of the present disclosure may comprise an ablation of at least nine nucleotide motifs relative to SEQ ID NO:132. In some embodiments, a CCL3 promoter of the present disclosure may comprise an ablation of ten or more nucleotide motifs relative to SEQ ID NO:132. In some embodiments, the nucleotide motifs are selected from Table 2. In certain embodiments, a CCL3 promoter of the present disclosure comprises ablation and substitution of all nucleotide motifs from Table 2, e.g., SEQ ID NO:169.

In some embodiments, a CCL3 promoter of the present disclosure may comprise an ablation of two nucleotide motifs relative to SEQ ID NO:132. In some embodiments, a CCL3 promoter of the present disclosure may comprise an ablation of three nucleotide motifs relative to SEQ ID NO:132. In some embodiments, a CCL3 promoter of the present disclosure may comprise an ablation of four nucleotide motifs relative to SEQ ID NO:132. In some embodiments, a CCL3 promoter of the present disclosure may comprise an ablation of five nucleotide motifs relative to SEQ ID NO:132. In some embodiments, a CCL3 promoter of the present disclosure may comprise an ablation of six nucleotide motifs relative to SEQ ID NO:132. In some embodiments, a CCL3 promoter of the present disclosure may comprise an ablation of seven nucleotide motifs relative to SEQ ID NO:132. In some embodiments, a CCL3 promoter of the present disclosure may comprise an ablation of eight nucleotide motifs relative to SEQ ID NO:132. In some embodiments, a CCL3 promoter of the present disclosure may comprise an ablation of nine nucleotide motifs relative to SEQ ID NO:132. In some embodiments, a CCL3 promoter of the present disclosure may comprise an ablation of ten nucleotide motifs relative to SEQ ID NO:132. In some embodiments, a CCL3 promoter of the present disclosure may comprise an ablation of eleven nucleotide motifs relative to SEQ ID NO:132. In some embodiments, a CCL3 promoter of the present disclosure may comprise an ablation of twelve nucleotide motifs relative to SEQ ID NO:132. In some embodiments, a CCL3 promoter of the present disclosure may comprise an ablation of thirteen nucleotide motifs relative to SEQ ID NO:132. In some embodiments, a CCL3 promoter of the present disclosure may comprise an ablation of fourteen nucleotide motifs relative to SEQ ID NO:132. In some embodiments, a CCL3 promoter of the present disclosure may comprise an ablation of fifteen nucleotide motifs relative to SEQ ID NO:132. In some embodiments, a CCL3 promoter of the present disclosure may comprise an ablation of sixteen nucleotide motifs relative to SEQ ID NO:132. In some embodiments, a CCL3 promoter of the present disclosure may comprise an ablation of seventeen nucleotide motifs relative to SEQ ID NO:132. In some embodiments, a CCL3 promoter of the present disclosure may comprise an ablation of eighteen nucleotide motifs relative to SEQ ID NO:132. In some embodiments, a CCL3 promoter of the present disclosure may comprise an ablation of nineteen nucleotide motifs relative to SEQ ID NO:132. In some embodiments, a CCL3 promoter of the present disclosure may comprise an ablation of twenty nucleotide motifs relative to SEQ ID NO:132. In some embodiments, a CCL3 promoter of the present disclosure may comprise an ablation of greater than twenty nucleotide motifs relative to SEQ ID NO:132. In some embodiments, the nucleotide motifs are selected from Table 2.

In some embodiments, a CCL3 promoter comprises at least one nucleotide motif. In some embodiments, a CCL3 promoter comprises at least two nucleotide motifs. In some embodiments, a CCL3 promoter comprises at least three nucleotide motifs. In some embodiments, a CCL3 promoter comprises at least four nucleotide motifs. In some embodiments, a CCL3 promoter comprises at least five nucleotide motifs. In some embodiments, a CCL3 promoter comprises at least six nucleotide motifs. In some embodiments, a CCL3 promoter comprises at least seven nucleotide motifs. In some embodiments, a CCL3 promoter comprises at least eight nucleotide motifs. In some embodiments, a CCL3 promoter comprises at least nine nucleotide motifs. In some embodiments, a CCL3 promoter comprises at least ten nucleotide motifs. In some embodiments, a CCL3 promoter comprises at least eleven nucleotide motifs. In some embodiments, a CCL3 promoter comprises at least twelve nucleotide motifs. In some embodiments, a CCL3 promoter comprises at least thirteen nucleotide motifs. In some embodiments, a CCL3 promoter comprises at least fourteen nucleotide motifs. In some embodiments, a CCL3 promoter comprises at least fifteen nucleotide motifs. In some embodiments, a CCL3 promoter comprises at least sixteen nucleotide motifs. In some embodiments, a CCL3 promoter comprises at least seventeen nucleotide motifs. In some embodiments, a CCL3 promoter comprises at least eighteen nucleotide motifs. In some embodiments, a CCL3 promoter comprises at least nineteen nucleotide motifs. In some embodiments, a CCL3 promoter comprises at least twenty nucleotide motifs. In some embodiments, the nucleotide motifs are selected from Table 2.

In some embodiments, a CCL3 promoter ablation variant of the present disclosure comprises the nucleotide sequence of SEQ ID NO:133. In some embodiments, a CCL3 promoter ablation variant of the present disclosure comprises the nucleotide sequence of SEQ ID NO:134. In some embodiments, a CCL3 promoter ablation variant of the present disclosure comprises the nucleotide sequence of SEQ ID NO:135. In some embodiments, a CCL3 promoter ablation variant of the present disclosure comprises the nucleotide sequence of SEQ ID NO:136. In some embodiments, a CCL3 promoter ablation variant of the present disclosure comprises the nucleotide sequence of SEQ ID NO:137. In some embodiments, a CCL3 promoter ablation variant of the present disclosure comprises the nucleotide sequence of SEQ ID NO:138. In some embodiments, a CCL3 promoter ablation variant of the present disclosure comprises the nucleotide sequence of SEQ ID NO:139. In some embodiments, a CCL3 promoter ablation variant of the present disclosure comprises the nucleotide sequence of SEQ ID NO:140. In some embodiments, a CCL3 promoter ablation variant of the present disclosure comprises the nucleotide sequence of SEQ ID NO:141. In some embodiments, a CCL3 promoter ablation variant of the present disclosure comprises the nucleotide sequence of SEQ ID NO:142. In some embodiments, a CCL3 promoter ablation variant of the present disclosure comprises the nucleotide sequence of SEQ ID NO:143. In some embodiments, a CCL3 promoter ablation variant of the present disclosure comprises the nucleotide sequence of SEQ ID NO:144. In some embodiments, a CCL3 promoter ablation variant of the present disclosure comprises the nucleotide sequence of SEQ ID NO:145. In some embodiments, a CCL3 promoter ablation variant of the present disclosure comprises the nucleotide sequence of SEQ ID NO:146. In some embodiments, a CCL3 promoter ablation variant of the present disclosure comprises the nucleotide sequence of SEQ ID NO:147. In some embodiments, a CCL3 promoter ablation variant of the present disclosure comprises the nucleotide sequence of SEQ ID NO:148. In some embodiments, a CCL3 promoter ablation variant of the present disclosure comprises the nucleotide sequence of SEQ ID NO:149. In some embodiments, a CCL3 promoter ablation variant of the present disclosure comprises the nucleotide sequence of SEQ ID NO:150. In some embodiments, a CCL3 promoter ablation variant of the present disclosure comprises the nucleotide sequence of SEQ ID NO:151. In some embodiments, a CCL3 promoter ablation variant of the present disclosure comprises the nucleotide sequence of SEQ ID NO:152. In some embodiments, a CCL3 promoter ablation variant of the present disclosure comprises the nucleotide sequence of SEQ ID NO:153. In some embodiments, a CCL3 promoter ablation variant of the present disclosure comprises the nucleotide sequence of SEQ ID NO:154. In some embodiments, a CCL3 promoter ablation variant of the present disclosure comprises the nucleotide sequence of SEQ ID NO:155. In some embodiments, a CCL3 promoter ablation variant of the present disclosure comprises the nucleotide sequence of SEQ ID NO:156. In some embodiments, a CCL3 promoter ablation variant of the present disclosure comprises the nucleotide sequence of SEQ ID NO:157. In some embodiments, a CCL3 promoter ablation variant of the present disclosure comprises the nucleotide sequence of SEQ ID NO:158. In some embodiments, a CCL3 promoter ablation variant of the present disclosure comprises the nucleotide sequence of SEQ ID NO:159. In some embodiments, a CCL3 promoter ablation variant of the present disclosure comprises the nucleotide sequence of SEQ ID NO:160. In some embodiments, a CCL3 promoter ablation variant of the present disclosure comprises the nucleotide sequence of SEQ ID NO:161. In some embodiments, a CCL3 promoter ablation variant of the present disclosure comprises the nucleotide sequence of SEQ ID NO:162. In some embodiments, a CCL3 promoter ablation variant of the present disclosure comprises the nucleotide sequence of SEQ ID NO:163. In some embodiments, a CCL3 promoter ablation variant of the present disclosure comprises the nucleotide sequence of SEQ ID NO:164. In some embodiments, a CCL3 promoter ablation variant of the present disclosure comprises the nucleotide sequence of SEQ ID NO:165. In some embodiments, a CCL3 promoter ablation variant of the present disclosure comprises the nucleotide sequence of SEQ ID NO: 166. In some embodiments, a CCL3 promoter ablation variant of the present disclosure comprises the nucleotide sequence of SEQ ID NO:167. In some embodiments, a CCL3 promoter ablation variant of the present disclosure comprises the nucleotide sequence of SEQ ID NO:168. In some embodiments, a CCL3 promoter ablation variant of the present disclosure comprises the nucleotide sequence of SEQ ID NO:169. In certain embodiments, an engineered CCL3 promoter of the present disclosure further comprises GGACAGAATTCCAAAGGCATGGTCGCACTTGGCTTCTGTCCTCTGTTATTCTCCAGC ATCAAATGTATCAACTCTAACCCCTTTG (SEQ ID NO:5) at the 5′ end.

In some embodiments, a CCL3 promoter ablation variant of the present disclosure does not comprise substitution at the ablation. In some embodiments, a CCL3 promoter ablation variant comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to SEQ ID NO:1. In some embodiments, a CCL3 promoter ablation variant of the present disclosure comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to SEQ ID NO:2. In some embodiments, a CCL3 promoter ablation variant of the present disclosure comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to SEQ ID NO:3. In some embodiments, a CCL3 promoter ablation variant of the present disclosure comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to SEQ ID NO:4. In some embodiments, a CCL3 promoter ablation variant of the present disclosure comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to SEQ ID NO:242. In some embodiments, a CCL3 promoter ablation variant of the present disclosure comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to SEQ ID NO:243. In some embodiments, a CCL3 promoter ablation variant of the present disclosure comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to SEQ ID NO:244. In some embodiments, a CCL3 promoter ablation variant of the present disclosure comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to SEQ ID NO:245. In some embodiments, a CCL3 promoter ablation variant of the present comprises a polynucleotide sequence having at least 85%, at least 90%, at least 91%, at least 92%, at least 93%, at least 94%, at least 95%, at least 96%, at least 97%, at least 98%, at least 99%, or 100% sequence identity to SEQ ID NO:246.

In some embodiments, a CCL3 promoter or CCL3 promoter ablation variant as described above may be operably linked to a nucleotide sequence encoding a polypeptide, e.g., an effector molecule described herein.

### Non-CCL3 Engineered Nucleic Acids

In another aspect, provided herein are engineered nucleic acids comprising: a first expression cassette comprising a first promoter and a first exogenous polynucleotide sequence encoding an activation-conditional control polypeptide (ACP) and/or an antigen recognizing receptor, wherein the first promoter is operably linked to the first exogenous polynucleotide; and a second expression cassette comprising an activation-conditional control polypeptide-responsive (ACP-responsive) promoter and a second exogenous polynucleotide sequence having the formula: (L-E)ₓ wherein E comprises a polynucleotide sequence encoding an effector molecule, L comprises a linker polynucleotide sequence, X=1 to 20, wherein the ACP-responsive promoter is operably linked to the second exogenous polynucleotide, wherein for the first iteration of the (L-E) unit, L is absent, and optionally wherein the ACP is capable of inducing expression of the first expression cassette by binding to the ACP-responsive promoter. In some embodiments, the ACP includes a drug-inducible domain, such as a tetracycline responsive domain (e.g., a TetR domain) or a repressible protease domain (e.g., an NS3 protease). In some embodiments, the ACP is a transcriptional modulator, e.g., a Zinc-finger protein. In some embodiments, the ACP is a small molecule (e.g., a drug) inducible peptide. In certain embodiments, the ACP is coupled to a promoter, e.g., an alternative promoter to the CCL3-variants described herein. Exemplary promoters are known in the art.

### Multicistronic and Multiple Promoter Systems

In some embodiments, engineered nucleic acids are configured to produce multiple effector molecules. For example, nucleic acids may be configured to produce 2-20 different effector molecules. In some embodiments, nucleic acids are configured to produce 2-20, 2-19, 2-18, 2-17, 2-16, 2-15, 2-14, 2-13, 2-12, 2-11, 2-10, 2-9, 2-8, 2-7, 2-6, 2-5, 2-4, 2-3, 3-20, 3-19, 3-18, 3-17, 3-16, 3-15, 3-14, 3-13, 3-12, 3-11, 3-10, 3-9, 3-8, 3-7, 3-6, 3-5, 3-4, 4-20, 4-19, 4-18, 4-17, 4-16, 4-15, 4-14, 4-13, 4-12, 4-11, 4-10, 4-9, 4-8, 4-7, 4-6, 4-5, 5-20, 5-19, 5-18, 5-17, 5-16, 5-15, 5-14, 5-13, 5-12, 5-11, 5-10, 5-9, 5-8, 5-7, 5-6, 6-20, 6-19, 6-18, 6-17, 6-16, 6-15, 6-14, 6-13, 6-12, 6-11, 6-10, 6-9, 6-8, 6-7, 7-20, 7-19, 7-18, 7-17, 7-16, 7-15, 7-14, 7-13, 7-12, 7-11, 7-10, 7-9, 7-8, 8-20, 8-19, 8-18, 8-17, 8-16, 8-15, 8-14, 8-13, 8-12, 8-11, 8-10, 8-9, 9-20, 9-19, 9-18, 9-17, 9-16, 9-15, 9-14, 9-13, 9-12, 9-11, 9-10, 10-20, 10-19, 10-18, 10-17, 10-16, 10-15, 10-14, 10-13, 10-12, 10-11, 11-20, 11-19, 11-18, 11-17, 11-16, 11-15, 11-14, 11-13, 11-12, 12-20, 12-19, 12-18, 12-17, 12-16, 12-15, 12-14, 12-13, 13-20, 13-19, 13-18, 13-17, 13-16, 13-15, 13-14, 14-20, 14-19, 14-18, 14-17, 14-16, 14-15, 15-20, 15-19, 15-18, 15-17, 15-16, 16-20, 16-19, 16-18, 16-17, 17-20, 17-19, 17-18, 18-20, 18-19, or 19-20 effector molecules. In some embodiments, nucleic acids are configured to produce 1, 2, 3, 4, 5, 6, 7, 8, 9, 10, 11, 12, 13, 14, 15, 16, 17, 18, 19, or 20 effector molecules.

In some embodiments, engineered nucleic acids can be multicistronic, i.e., more than one separate polypeptide (e.g., multiple exogenous polynucleotides or effector molecules) can be produced from a single mRNA transcript. Engineered nucleic acids can be multicistronic through the use of various linkers, e.g., a polynucleotide sequence encoding a first exogenous polynucleotide or effector molecule can be linked to a nucleotide sequence encoding a second exogenous polynucleotide or effector molecule, such as in a first gene:linker:second gene 5′ to 3′ orientation. A linker polynucleotide sequence can encode a 2A ribosome skipping element, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage.

In some embodiments, when the second expression cassette comprises two or more units of (L₁−E)ₓ, each L₁ linker polynucleotide sequence is operably associated with the translation of each effector molecule as a separate polypeptide.

A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker is a Furin-Gly-Ser-Gly-2A fusion polypeptide. In some embodiments, a linker is a Furin-Gly-Ser-Gly-T2A fusion polypeptide.

In general, a multicistronic system can use any number or combination of linkers, to express any number of genes or portions thereof (e.g., an engineered nucleic acid can encode a first, a second, and a third effector molecule, each separated by linkers such that separate polypeptides encoded by the first, second, and third effector molecules are produced).

“Linkers,” as used herein can refer to polypeptides that link a first polypeptide sequence and a second polypeptide sequence or the multicistronic linkers described above.

### Effector Molecules

Any suitable effector molecule known in the art can be encoded by the engineered nucleic acid or expressed by the engineered cell. Suitable effector molecules can be grouped into therapeutic classes based on structure similarity, sequence similarity, or function. Effector molecule therapeutic classes include, but are not limited to, cytokines, chemokines, homing molecules, growth factors, co-activation molecules, tumor microenvironment modifiers, receptors, ligands, antibodies, polynucleotides, peptides, and enzymes.

In some embodiments, each effector molecule is independently selected from a therapeutic class, wherein the therapeutic class is selected from: a cytokine, a chemokine, a homing molecule, a growth factor, a co-activation molecule, a tumor microenvironment modifier a, a receptor, a ligand, an antibody, a polynucleotide, a peptide, and an enzyme.

In some embodiments, each effector molecule is independently selected from a therapeutic class, wherein the therapeutic class is selected from: a cytokine, a chemokine, a homing molecule, a growth factor, a co-activation molecule, a tumor microenvironment modifier a, a receptor, a ligand, an antibody, a peptide, and an enzyme.

In some embodiments, a effector molecule is a chemokine. Chemokines are small cytokines or signaling proteins secreted by cells that can induce directed chemotaxis in cells. Chemokines can be classified into four main subfamilies: CXC, CC, CX3C and XC, all of which exert biological effects by binding selectively to chemokine receptors located on the surface of target cells. Non-limiting examples of chemokines that may be encoded by the engineered nucleic acids of the present disclosure include: CCL21a, CXCL10, CXCL11, CXCL13, a CXCL10-CXCL11 fusion protein, CCL19, CXCL9, and XCL1, or any combination thereof. In some embodiments, the chemokine is selected from: CCL21a, CXCL10, CXCL11, CXCL13, a CXCL10-CXCL11 fusion protein, CCL19, CXCL9, and XCL1.

In some embodiments, a effector molecule is a cytokine. Non-limiting examples of cytokines that may be encoded by the engineered nucleic acids of the present disclosure include: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha, or any combination thereof. In some embodiments, the cytokine is selected from: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.

In some embodiments, engineered nucleic acids are configured to produce at least one homing molecule. “Homing,” refers to active navigation (migration) of a cell to a target site (e.g., a cell, tissue (e.g., tumor), or organ). A “homing molecule” refers to a molecule that directs cells to a target site. In some embodiments, a homing molecule functions to recognize and/or initiate interaction of an engineered cell to a target site. Non-limiting examples of homing molecules include CXCR1, CCR9, CXCR2, CXCR3, CXCR4, CCR2, CCR4, FPR2, VEGFR, IL6R, CXCR1, CSCR7, PDGFR, anti-integrin alpha4, beta7; anti-MAdCAM; CCR9; CXCR4; SDFI; MMP-2; CXCR1; CXCR7; CCR2; CCR4; and GPR15, or any combination thereof. In some embodiments, the homing molecule is selected from: anti-integrin alpha4, beta7; anti-MAdCAM; CCR9; CXCR4; SDFI; MMP-2; CXCR1; CXCR7; CCR2; CCR4; and GPR15.

In some embodiments, engineered nucleic acids are configured to produce at least one growth factor. Suitable growth factors for use as an effector molecule include, but are not limited to, FLT3L and GM-CSF, or any combination thereof. In some embodiments, the growth factor is selected from: FLT3L and GM-CSF.

In some embodiments, engineered nucleic acids are configured to produce at least one co-activation molecule. Suitable co-activation molecules for use as an effector molecule include, but are not limited to, c-Jun, 4-1BBL and CD40L, or any combination thereof. In some embodiments, the co-activation molecule is selected from: c-Jun, 4-1BBL and CD40L.

A “tumor microenvironment” is the cellular environment in which a tumor exists, including surrounding blood vessels, immune cells, fibroblasts, bone marrow-derived inflammatory cells, lymphocytes, signaling molecules and the extracellular matrix (ECM) (see, e.g., Pattabiraman, D. R. & Weinberg, R. A. Nature Reviews Drug Discovery 13, 497-512 (2014); Balkwill, F. R. et al. J Cell Sci 125, 5591-5596, 2012; and Li, H. et al. J Cell Biochem 101(4), 805-15, 2007). Suitable tumor microenvironment modifiers for use as an effector molecule include, but are not limited to, adenosine deaminase, TGFbeta inhibitors, immune checkpoint inhibitors, VEGF inhibitors, and HPGE2, or any combination thereof. In some embodiments, the tumor microenvironment modifier is selected from: adenosine deaminase, TGFbeta inhibitors, immune checkpoint inhibitors, VEGF inhibitors, and HPGE2.

In some embodiments, engineered nucleic acids are configured to produce at least one TGFbeta inhibitor. Suitable TGFbeta inhibitors for use as an effector molecule include, but are not limited to, an anti-TGFbeta peptide, an anti-TGFbeta antibody, a TGFb-TRAP, or combinations thereof. In some embodiments, the TGFbeta inhibitors are selected from: an anti-TGFbeta peptide, an anti-TGFbeta antibody, a TGFb-TRAP, and combinations thereof.

In some embodiments, engineered nucleic acids are configured to produce at least one immune checkpoint inhibitor. Suitable immune checkpoint inhibitors for use as an effector molecule include, but are not limited to, anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GAL9 antibodies, anti-A2AR antibodies, anti-phosphatidylserine antibodies, anti-CD27 antibodies, anti-TNFa antibodies, anti-TREM1 antibodies, and anti-TREM2 antibodies, or any combination thereof. In some embodiments, the immune checkpoint inhibitors are selected from: anti-PD-1 antibodies, anti-PD-L1 antibodies, anti-PD-L2 antibodies, anti-CTLA-4 antibodies, anti-LAG-3 antibodies, anti-TIM-3 antibodies, anti-TIGIT antibodies, anti-VISTA antibodies, anti-KIR antibodies, anti-B7-H3 antibodies, anti-B7-H4 antibodies, anti-HVEM antibodies, anti-BTLA antibodies, anti-GAL9 antibodies, anti-A2AR antibodies, anti-phosphatidylserine antibodies, anti-CD27 antibodies, anti-TNFa antibodies, anti-TREM1 antibodies, and anti-TREM2 antibodies.

Illustrative immune checkpoint inhibitors include pembrolizumab (anti-PD-1; MK-3475/Keytruda®—Merck), nivolumamb (anti-PD-1; Opdivo®—BMS), pidilizumab (anti-PD-1 antibody; CT-011—Teva/CureTech), AMP224 (anti-PD-1; NC), avelumab (anti-PD-L1; Bavencio®—Pfizer), durvalumab (anti-PD-L1; MEDI4736/Imfinzi®—Medimmune/AstraZeneca), atezolizumab (anti-PD-L1; Tecentriq®—Roche/Genentech), BMS—936559 (anti-PD-L1—BMS), tremelimumab (anti-CTLA-4; Medimmune/AstraZeneca), ipilimumab (anti-CTLA-4; Yervoy®—BMS), lirilumab (anti-KIR; BMS), monalizumab (anti-NKG2A; Innate Pharma/AstraZeneca).

In some embodiments, engineered nucleic acids are configured to produce at least one VEGF inhibitor. Suitable VEGF inhibitors for use as an effector molecule include, but are not limited to, anti-VEGF antibodies, anti-VEGF peptides, or combinations thereof. In some embodiments, the VEGF inhibitors comprise anti-VEGF antibodies, anti-VEGF peptides, or combinations thereof.

In some embodiments, each effector molecule is a human-derived effector molecule.

### Secretion Signals

In general, the one or more effector molecules comprise a secretion signal peptide (also referred to as a signal peptide or signal sequence) at the effector molecule's N-terminus that direct newly synthesized proteins destined for secretion or membrane insertion to the proper protein processing pathways. In embodiments with two or more effector molecules, each effector molecule can comprise a secretion signal (S). In embodiments with two or more effector molecules, each effector molecule can comprise a secretion signal such that each effector molecule is secreted from an engineered cell. In embodiments, the second expression cassette comprising one or more units of (L-E)ₓ further comprises a polynucleotide sequence encoding a secretion signal peptide (S). In embodiments, for each X the corresponding secretion signal peptide is operably associated with the effector molecule. In embodiments, the second expression cassette comprising an ACP-responsive promoter and a second exogenous polynucleotide sequence having the formula: (L-S-E)ₓ.

The secretion signal peptide operably associated with a effector molecule can be a native secretion signal peptide native secretion signal peptide (e.g., the secretion signal peptide generally endogenously associated with the given effector molecule). The secretion signal peptide operably associated with a effector molecule can be a non-native secretion signal peptide native secretion signal peptide. Non-native secretion signal peptides can promote improved expression and function, such as maintained secretion, in particular environments, such as tumor microenvironments. Non-limiting examples of non-native secretion signal peptide are shown in Table 3.

TABLE 3
Exemplary Signal Secretion Peptides

| | | Source | |
|---|---|---|---|
| Name | Protein SEQUENCE | (Uniprot) | DNA SEQUENCE |
| IL-12 | MCHQQLVISWFSL | P29460 | ATGTGTCACCAGCAGCTCGTTAT |
| | VFLASPLVA (SEQ | | ATCCTGGTTTAGTTTGGTGTTTCT |
| | ID NO: 56) | | CGCTTCACCCCTGGTGGCA (SEQ |
| | | | ID NO: 31) |
| IL-12 (Codon | MCHQQLVISWFSL | — | ATGTGCCATCAGCAACTCGTCAT |
| Optimized) | VFLASPLVA (SEQ | | CTCCTGGTTCTCCCTTGTGTTCCT |
| | ID NO: 57) | | CGCTTCCCCTCTGGTCGCC (SEQ |
| | | | ID NO: 32) |
| IL-2 (Optimized) | MQLLSCIALILALV | — | ATGCAACTGCTGTCATGTATCGC |
| | (SEQ ID NO: 58) | | ACTCATCCTGGCGCTGGTA (SEQ |
| | | | ID NO: 33) |
| IL-2 (Native) | MYRMQLLSCIALSL | P60568 | ATGTATCGGATGCAACTTTTGAG |
| | ALVTNS (SEQ ID | | CTGCATCGCATTGTCTCTGGCGCT |
| | NO: 59) | | GGTGACAAATTCC (SEQ ID NO: |
| | | | 34) |
| Trypsinogen-2 | MNLLLILTFVAAAV | P07478 | ATGAATCTCTTGCTCATACTTACG |
| | A (SEQ ID NO: 60) | | TTTGTCGCTGCTGCCGTTGCG |
| | | | (SEQ ID NO: 35) |
| Gaussia | MGVKVLFALICIAV | — | ATGGGCGTGAAGGTCTTGTTTGC |
| Luciferase | AEA (SEQ ID NO: | | CCTTATCTGCATAGCTGTTGCGGA |
| | 61) | | GGCG (SEQ ID NO: 36) |
| CD5 | MPMGSLQPLATLY | P06127 | ATGCCGATGGGGAGCCTTCAACC |
| | LLGMLVASCLG | | TTTGGCAACGCTTTATCTTCTGGG |
| | (SEQ ID NO: 62) | | GATGTTGGTTGCTAGTTGCCTTGG |
| | | | G (SEQ ID NO: 37) |
| IgKVII (mouse) | METDTLLLWVLLL | | ATGGAAACTGACACGTTGTTGCT |
| | WVPGSTGD (SEQ | | GTGGGTATTGCTCTTGTGGGTCCC |
| | ID NO: 63) | | AGGATCTACGGGCGAC (SEQ ID |
| | | | NO: 38) |
| IgKVII (human) | MDMRVPAQLLGLL | P01597 | ATGGATATGAGGGTTCCCGCCCA |
| | LLWLRGARC (SEQ | | GCTTTTGGGGCTGCTTTTGTTGTG |
| | ID NO: 64) | | GCTTCGAGGGGCTCGGTGT (SEQ |
| | | | ID NO: 39) |
| VSV-G | MKCLLYLAFLFIGV | — | ATGAAGTGTCTGTTGTACCTGGC |
| | NC (SEQ ID NO: 65) | | GTTTCTGTTCATTGGTGTAAACTG |
| | | | T (SEQ ID NO: 40) |
| Prolactin | MNIKGSPWKGSLLL | P01236 | ATGAATATCAAAGGAAGTCCGTG |
| | LLVSNLLLCQSVAP | | GAAGGGTAGTCTCCTGCTGCTCCT |
| | (SEQ ID NO: 66) | | CGTATCTAACCTTCTCCTTTGTCA |
| | | | ATCCGTGGCACCC (SEQ ID NO: 41) |
| Serum albumin | MKWVTFISLLFLFS | P02768 | ATGAAATGGGTAACATTCATATC |
| preproprotein | SAYS (SEQ ID NO: | | ACTTCTCTTTCTGTTCAGCTCTGC |
| | 67) | | GTATTCT (SEQ ID NO: 42) |
| Azurocidin | MTRLTVLALLAGL | 20160 | ATGACAAGGCTTACTGTTTTGGCT |
| preproprotein | LASSRA (SEQ ID | | CTCCTCGCTGGACTCTTGGCTTCC |
| | NO: 68) | | TCCCGAGCA (SEQ ID NO: 43) |
| Osteonectin | MRAWIFFLLCLAGR | P09486 | ATGAGGGCTTGGATTTTTTTTCTG |
| (BM40) | ALA (SEQ ID NO: | | CTCTGCCTTGCCGGTCGAGCCCTG |
| | 69) | | GCG (SEQ ID NO: 44) |
| CD33 | MPLLLLLPLLWAG | P20138 | ATGCCTCTTCTGCTTTTGCTTCCT |
| | ALA (SEQ ID NO: | | CTTTTGTGGGCAGGTGCCCTCGCA |
| | 70) | | (SEQ ID NO: 45) |
| IL-6 | MNSFSTSAFGPVAF | P05231 | ATGAACTCTTTCTCAACCTCTGCG |
| | SLGLLLVLPAAFPA | | TTTGGTCCGGTCGCTTTCTCCCTT |
| | P (SEQ ID NO: 71) | | GGGCTCCTGCTTGTCTTGCCAGCA |
| | | | GCGTTTCCTGCGCCA (SEQ ID NO: |
| | | | 46) |
| IL-8 | MTSKLAVALLAAF | P10145 | ATGACAAGTAAACTGGCGGTAGC |
| | LISAALC (SEQ ID | | CTTGCTCGCGGCCTTTTTGATTTC |
| | NO: 72) | | CGCAGCCCTTTGT (SEQ ID NO: 47) |
| CCL2 | MKVSAALLCLLLIA | P13500 | ATGAAGGTAAGTGCAGCGTTGCT |
| | ATFIPQGLA (SEQ | | TTGCCTTCTCCTCATTGCAGCGAC |
| | ID NO: 73) | | CTTTATTCCTCAAGGGCTGGCC |
| | | | (SEQ ID NO: 48) |
| TIMP2 | MGAAARTLRLALG | P16035 | ATGGGAGCGGCAGCTAGAACACT |
| | LLLLATLLRPADA | | TCGACTTGCCCTTGGGCTCTTGCT |
| | (SEQ ID NO: 74) | | CCTTGCAACCCTCCTTAGACCTGC |
| | | | CGACGCA (SEQ ID NO: 49) |
| VEGFB | MSPLLRRLLLAALL | P49765 | ATGTCACCGTTGTTGCGGAGATT |
| | QLAPAQA (SEQ ID | | GCTGTTGGCCGCACTTTTGCAACT |
| | NO: 75) | | GGCTCCTGCTCAAGCC (SEQ ID |
| | | | NO: 50) |
| Osteoprotegerin | MNNLLCCALVFLDI | O00300 | ATGAATAACCTGCTCTGTTGTGCG |
| | SIKWTTQ (SEQ ID | | CTCGTGTTCCTGGACATTTCTATA |
| | NO: 76) | | AAATGGACAACGCAA (SEQ ID |
| | | | NO: 51) |
| Serpin E1 | MQMSPALTCLVLG | P05121 | ATGCAAATGTCTCCTGCCCTTACC |
| | LALVFGEGSA (SEQ | | TGTCTCGTACTTGGTCTTGCGCTC |
| | ID NO: 77) | | GTATTTGGAGAGGGATCAGCC |
| | | | (SEQ ID NO: 52) |
| GROalpha | MARAALSAAPSNP | P09341 | ATGGCAAGGGCTGCACTCAGTGC |
| | RLLRVALLLLLLVA | | TGCCCCGTCTAATCCCAGATTGCT |
| | AGRRAAG (SEQ ID | | TCGAGTTGCATTGCTTCTTCTGTT |
| | NO: 78) | | GCTGGTTGCAGCTGGTAGGAGAG |
| | | | CAGCGGGT (SEQ ID NO: 53) |
| CXCL12 | MNAKVVVVLVLVL | P48061 | ATGAATGCAAAAGTCGTGGTCGT |
| | TALCLSDG (SEQ ID | | GCTGGTTTTGGTTCTGACGGCGTT |
| | NO: 79) | | GTGTCTTAGTGATGGG (SEQ ID |
| | | | NO: 54) |
| IL-21 (Codon | MERIVICLMVIFLGT | Q9HBE4 | ATGGAACGCATTGTGATCTGCCT |
| Optimized) | LVHKSSS (SEQ ID | | GATGGTCATCTTCCTGGGCACCTT |
| | NO: 80) | | AGTGCACAAGTCGAGCAGC (SEQ |
| | | | ID NO: 55) |
| CD8a | MALPVTALLLPLAL | P01732 | ATGGCTCTCCCTGTAACTGCCCTG |
| | LLHAARP | | CTTCTTCCCCTTGCCTTGCTTCTCC |
| | (SEQ ID NO: 83) | | ATGCCGCTAGACCC (SEQ ID NO: |
| | | | 84) |
| GMCSFR | MLLLVTSLLLCELP | P15509 | ATGCTGCTGCTGGTCACATCTCTG |
| | HPAFLLIP | | CTGCTGTGCGAGCTGCCCCATCCT |
| | (SEQ ID NO: 85) | | GCCTTTCTGCTGATCCCT (SEQ ID |
| | | | NO: 86) |
| | | | ATGCTGCTGCTGGTTACATCTCTG |
| | | | CTGCTGTGCGAGCTGCCCCATCCT |
| | | | GCCTTTCTGCTGATCCCT (SEQ ID |
| | | | NO: 87) |

### Antigen Recognizing Receptors

Certain aspects of the present disclosure relate to an engineered nucleic comprising an antigen recognizing receptor. In some embodiments, an engineered nucleic acid of the present disclosure comprises a first expression cassette that further comprises an antigen recognizing receptor. In some embodiments, the first expression cassette comprises a polynucleotide sequence encoding the antigen recognizing receptor that is operably linked to the first exogenous polynucleotide sequence encoding the ACP and to the first promoter. Suitable antigen recognizing receptors for use as an effector molecule recognize antigens that include, but are not limited to, 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD70, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra, IL-13Ra2, IL-8, IL-15, IL1RAP, Integrin aV, KIT, L1CAM, LAMP1, Lewis Y, LeY, LIV-1, LRRC, LY6E, MCSP, Mesothelin (MSLN), MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1, or any combination thereof.

In some embodiments, the antigen recognizing receptor recognizes an antigen selected from: 5T4, ADAM9, AFP, AXL, B7-H3, B7-H4, B7-H6, C4.4, CA6, Cadherin 3, Cadherin 6, CCR4, CD123, CD133, CD138, CD142, CD166, CD25, CD30, CD352, CD37, CD38, CD44, CD56, CD66e, CD70, CD71, CD74, CD79b, CD80, CEA, CEACAM5, Claudin18.2, cMet, CSPG4, CTLA, DLK1, DLL3, DR5, EGFR, ENPP3, EpCAM, EphA2, Ephrin A4, ETBR, FGFR2, FGFR3, FRalpha, FRb, GCC, GD2, GFRa4, gpA33, GPC3, gpNBM, GPRC5, HER2, IL-13R, IL-13Ra, IL-13Ra2, IL-8, IL-15, IL1RAP, Integrin aV, KIT, L1CAM, LAMP1, Lewis Y, LeY, LIV-1, LRRC, LY6E, MCSP, Mesothelin, MUC1, MUC16, MUC1C, NaPi2B, Nectin 4, NKG2D, NOTCH3, NY ESO 1, Ovarin, P-cadherin, pan-Erb2, PSCA, PSMA, PTK7, ROR1, S Aures, SCT, SLAMF7, SLITRK6, SSTR2, STEAP1, Survivin, TDGF1, TIM1, TROP2, and WT1.

In some embodiments, the first expression cassette further comprises a linker polynucleotide sequence localized between the ACP and the antigen recognizing receptor.

In some embodiments, the antigen recognizing receptor comprises an antigen-binding domain. In some embodiments, the antigen-binding domain comprises an antibody, an antigen-binding fragment of an antibody, a F(ab) fragment, a F(ab′) fragment, a single chain variable fragment (scFv), or a single-domain antibody (sdAb). In some embodiments, the antigen-binding domain comprises a single chain variable fragment (scFv). In some embodiments, the scFv comprises a heavy chain variable domain (VH) and a light chain variable domain (VL). In some embodiments, the VH and VL are separated by a peptide linker.

An scFv has a variable domain of light chain (VL) connected from its C-terminus to the N-terminal end of a variable domain of heavy chain (VH) by a polypeptide chain. Alternately the scFv comprises of polypeptide chain where in the C-terminal end of the VH is connected to the N-terminal end of VL by a polypeptide chain. In some embodiments, the scFv comprises the structure VH-L-VL or VL-L-VH, wherein VH is the heavy chain variable domain, L is the peptide linker, and VL is the light chain variable domain.

An sdAb is a molecule in which one variable domain of an antibody specifically binds to an antigen without the presence of the other variable domain.

A F(ab) fragment contains the constant domain (CL) of the light chain and the first constant domain (CH1) of the heavy chain along with the variable domains VL and VH on the light and heavy chains respectively. F(ab′) fragments differ from Fab fragments by the addition of a few residues at the carboxy terminus of the heavy chain CH1 domain including one or more cysteines from the antibody hinge region. F(ab′)₂ fragments contain two Fab′ fragments joined, near the hinge region, by disulfide bonds.

In some embodiments, the antigen recognizing receptor is a chimeric antigen receptor (CAR) or T cell receptor (TCR). In some embodiments, the antigen recognizing receptor is a CAR. In some embodiments, the CAR comprises one or more intracellular signaling domains, and the one or more intracellular signaling domains are selected from: a CD3zeta-chain intracellular signaling domain, a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, and a MyD88 intracellular signaling domain. In some embodiments, the CAR comprises a CD3zeta-chain intracellular signaling domain and one or more additional intracellular signaling domains (e.g., co-stimulatory domains) selected from a CD97 intracellular signaling domain, a CD11a-CD18 intracellular signaling domain, a CD2 intracellular signaling domain, an ICOS intracellular signaling domain, a CD27 intracellular signaling domain, a CD154 intracellular signaling domain, a CD8 intracellular signaling domain, an OX40 intracellular signaling domain, a 4-1BB intracellular signaling domain, a CD28 intracellular signaling domain, a ZAP40 intracellular signaling domain, a CD30 intracellular signaling domain, a GITR intracellular signaling domain, an HVEM intracellular signaling domain, a DAP10 intracellular signaling domain, a DAP12 intracellular signaling domain, a MyD88 intracellular signaling domain, a 2B4 intracellular signaling domain, a CD16a intracellular signaling domain, a DNAM-1 intracellular signaling domain, a KIR2DS1 intracellular signaling domain, a KIR3DS1 intracellular signaling domain, a NKp44 intracellular signaling domain, a NKp46 intracellular signaling domain, a FceR1g intracellular signaling domain, a NKG2D intracellular signaling domain, and an EAT-2 intracellular signaling domain.

In some embodiments, the CAR further comprises a transmembrane domain, and the transmembrane domain is selected from: a CD8 transmembrane domain, a CD28 transmembrane domain a CD3zeta-chain transmembrane domain, a CD4 transmembrane domain, a 4-1BB transmembrane domain, an OX40 transmembrane domain, an ICOS transmembrane domain, a CTLA-4 transmembrane domain, a PD-1 transmembrane domain, a LAG-3 transmembrane domain, a 2B4 transmembrane domain, a BTLA transmembrane domain, an OX40 transmembrane domain, a DAP10 transmembrane domain, a DAP12 transmembrane domain, a CD16a transmembrane domain, a DNAM-1 transmembrane domain, a KIR2DS1 transmembrane domain, a KIR3DS1 transmembrane domain, an NKp44 transmembrane domain, an NKp46 transmembrane domain, an FceR1g transmembrane domain, and an NKG2D transmembrane domain.

In some embodiments, the CAR further comprises a spacer region (e.g., hinge domain) between the antigen-binding domain and the transmembrane domain. A spacer or hinge domain is any oligopeptide or polypeptide that functions to link the transmembrane domain to the extracellular domain and/or the intracellular signaling domain in the polypeptide chain. Spacer or hinge domains provide flexibility to the inhibitory chimeric receptor or tumor-targeting chimeric receptor, or domains thereof, or prevent steric hindrance of the inhibitory chimeric receptor or tumor-targeting chimeric receptor, or domains thereof. In some embodiments, a spacer domain or hinge domain may comprise up to 300 amino acids (e.g., 10 to 100 amino acids, or 5 to 20 amino acids). In some embodiments, one or more spacer domain(s) may be included in other regions of an inhibitory chimeric receptor or tumor-targeting chimeric receptor.

Exemplary spacer or hinge domains may include, without limitation an IgG domain (such as an IgG1 hinge, an IgG2 hinge, an IgG3 hinge, or an IgG4 hinge), an IgD hinge domain, a CD8a hinge domain, and a CD28 hinge domain. In some embodiments, the spacer or hinge domain is an IgG domain, an IgD domain, a CD8a hinge domain, or a CD28 hinge domain.

Exemplary spacer or hinge domain protein sequences are shown in Table 4. Exemplary spacer or hinge domain nucleotide sequences are shown in Table 5.

TABLE 4

| Amino Acid Sequence | SEQ ID NO: | Description |
|---|---|---|
| AAAIEVMYPPPYLDNEKSNGTIIHVKGKHLCPSPLFP | 100 | CD28 hinge |
| GPSKP | | |
| ESKYGPPCPSCP | 101 | IgG4 minimal hinge |
| ESKYGPPAPSAP | 102 | IgG4 minimal hinge, no disulfides |
| ESKYGPPCPPCP | 103 | IgG4 S228P minimal hinge, |
| | | enhanced disulfide formation |
| EPKSCDKTHTCP | 104 | IgG1 minimal hinge |
| AAAFVPVFLPAKPTTTPAPRPPTPAPTIASQPLSLRPE | 105 | Extended CD8a hinge |
| ACRPAAGGAVHTRGLDFACDIYIWAPLAGTCGVLL | | |
| LSLVITLYCNHRN | | |
| TTTPAPRPPTPAPTIALQPLSLRPEACRPAAGGAVHT | 106 | CD8a hinge |
| RGLDFACD | | |
| ACPTGLYTHSGECCKACNLGEGVAQPCGANQTVCE | 107 | LNGFR hinge |
| PCLDSVTFSDVVSATEPCKPCTECVGLQSMSAPCVE | | |
| ADDAVCRCAYGYYQDETTGRCEACRVCEAGSGLV | | |
| FSCQDKQNTVCEECPDGTYSDEADAEC | | |
| ACPTGLYTHSGECCKACNLGEGVAQPCGANQTVC | 108 | Truncated LNGFR hinge (TNFR- |
| | | Cys1) |
| AVGODTQEVIVVPHSLPFKV | 109 | PDGFR-beta extracellular linker |

TABLE 5

| Nucleic Acid Sequence | SEQ ID NO: | Description |
|---|---|---|
| GCAGCAGCTATCGAGGTGATGTATCCTCCGCCCTACC | 110 | CD28 hinge |
| TGGATAATGAAAAGAGTAATGGGACTATCATTCATGT | | |
| AAAAGGGAAGCATCTTTGTCCTTCTCCCCTTTTCCCC | | |
| GGTCCGTCTAAACCT | | |
| GAAAGCAAGTACGGTCCACCTTGCCCTAGCTGTCCG | 111 | IgG4 minimal hinge |
| GAATCCAAGTACGGCCCCCCAGCGCCTAGTGCCCCA | 112 | IgG4 minimal hinge, no |
| | | disulfides |
| GAATCTAAATATGGCCCGCCATGCCCGCCTTGCCCA | 113 | IgG4 S228P minimal hinge, |
| | | enhanced disulfide formation |
| GAACCGAAGTCTTGTGATAAAACTCATACGTGCCCG | 114 | IgG1 minimal hinge |
| GCTGCTGCTTTCGTACCCGTGTTCCTCCCTGCTAAGCC | 115 | Extended CD8a hinge |
| TACGACTACCCCCGCACCGAGACCACCCACGCCAGC | | |
| ACCCACGATTGCTAGCCAGCCCCTTAGTTTGCGACCA | | |
| GAAGCTTGTCGGCCTGCTGCTGGTGGCGCGGTACATA | | |
| CCCGCGGCCTTGATTTTGCTTGCGATATATATATCTG | | |
| GGCGCCTCTGGCCGGAACATGCGGGGTCCTCCTCCTT | | |
| TCTCTGGTTATTACTCTCTACTGTAATCACAGGAAT | | |
| GCCTGCCCGACCGGGCTCTACACTCATAGCGGGGAAT | 116 | LNGFR hinge |
| GTTGTAAGGCATGTAACTTGGGTGAGGGCGTCGCAC | | |
| AGCCCTGCGGAGCTAACCAAACAGTGTGCGAACCCT | | |
| GCCTCGATAGTGTGACGTTCTCTGATGTTGTATCAGC | | |
| TACAGAGCCTTGCAAACCATGTACTGAGTGCGTTGGA | | |
| CTTCAGTCAATGAGCGCTCCATGTGTGGAGGCAGATG | | |
| ATGCGGTCTGTCGATGTGCTTACGGATACTACCAAGA | | |
| CGAGACAACAGGGCGGTGCGAGGCCTGTAGAGTTTG | | |
| TGAGGCGGGCTCCGGGCTGGTGTTTTCATGTCAAGAC | | |
| AAGCAAAATACGGTCTGTGAAGAGTGCCCTGATGGC | | |
| ACCTACTCAGACGAAGCAGATGCAGAATGC | | |
| GCCTGCCCTACAGGACTCTACACGCATAGCGGTGAGT | 117 | Truncated LNGFR hinge |
| GTTGTAAAGCATGCAACCTCGGGGAAGGTGTAGCCC | | (TNFR-Cys1) |
| AGCCATGCGGGGCTAACCAAACCGTTTGC | | |
| GCTGTGGGCCAGGACACGCAGGAGGTCATCGTGGTG | 118 | PDGFR-beta extracellular |
| CCACACTCCTTGCCCTTTAAGGTG | | linker |

Suitable transmembrane domains, spacer or hinge domains, and intracellular domains for use in a CAR are generally described in Stoiber et al, Cells 2019, 8(5), 472; Guedan et al, Mol Therapy: Met & Clinic Dev, 2019 12:145-156; and Sadelain et al, Cancer Discov; 2013, 3(4); 388-98, each of which are hereby incorporated by reference in their entirety.

In some embodiments, the CAR further comprises a secretion signal peptide. Any suitable secretion signal peptide of the present disclosure may be used.

### Post-Transcriptional Regulatory Elements

In some embodiments, an engineered nucleic acid of the present disclosure comprises a post-transcriptional regulatory element (PRE). PREs can enhance gene expression via enabling tertiary RNA structure stability and 3′ end formation. Non-limiting examples of PREs include the Hepatitis B virus PRE (HPRE) and the Woodchuck Hepatitis Virus PRE (WPRE). In some embodiments, the post-transcriptional regulatory element is a Woodchuck Hepatitis Virus Posttranscriptional Regulatory Element (WPRE). In some embodiments, the WPRE comprises the alpha, beta, and gamma components of the WPRE element. In some embodiments, the WPRE comprises the alpha component of the WPRE element.

### Immunoresponsive Cells

Certain aspects of the present disclosure relate to a cell, such as an immunoresponsive cell, that has been genetically engineered to comprise one or more chimeric receptors of the present disclosure or one or more polynucleotides encoding such chimeric receptors, and to methods of using such cells for treating solid tumors.

In some embodiments, the cell is a mammalian cell. In some embodiments, the mammalian cell is a primary cell. In some embodiments, the mammalian cell is a cell line. In some embodiments, the mammalian cell a bone marrow cell, a blood cell, a skin cell, bone cell, a muscle cell, a neuronal cell, a fat cell, a liver cell, or a heart cell. In some embodiments, the cell is a stem cell. Exemplary stem cells include, without limitation embryonic stem cells (ESCs), induced pluripotent stem cells (iPSCs), adult stem cells, and tissue-specific stem cells, such as hematopoietic stem cells (blood stem cells), mesenchymal stem cells (MSC), neural stem cells, epithelial stem cells, or skin stem cells. In some embodiments, the cell is a cell that is derived or differentiated from a stem cell of the present disclosure. In some embodiments, the cell is an immune cell. Immune cells of the present disclosure may be isolated or differentiated from a stem cell of the present disclosure (e.g., from an ESC or iPSC). Exemplary immune cells include, without limitation, T cells (e.g., helper T cells, cytotoxic T cells, memory T cells, regulatory T cells, natural killer T cells, alpha beta T cells, and gamma delta T cells), B cells, natural killer (NK) cells, dendritic cells, myeloid cells, macrophages, and monocytes. In some embodiments, the cell is a neuronal cell. Neuronal cells of the present disclosure may be isolated or differentiated from a stem cell of the present disclosure (e.g., from an ESC or iPSC). Exemplary neuronal cells include, without limitation, neural progenitor cells, neurons (e.g., sensory neurons, motor neurons, cholinergic neurons, GABAergic neurons, glutamatergic neurons, dopaminergic neurons, or serotonergic neurons), astrocytes, oligodendrocytes, and microglia.

In some embodiments, the cell is an immunoresponsive cell. Immunoresponsive cells of the present disclosure may be isolated or differentiated from a stem cell of the present disclosure (e.g., from an ESC or iPSC). Exemplary immunoresponsive cells of the present disclosure include, without limitation, cells of the lymphoid lineage. The lymphoid lineage, comprising B cells, T cells, and natural killer (NK) cells, provides for the production of antibodies, regulation of the cellular immune system, detection of foreign agents in the blood, detection of cells foreign to the host, and the like. Examples of immunoresponsive cells of the lymphoid lineage include, without limitation, T cells, Natural Killer (NK) cells, embryonic stem cells, pluripotent stem cells, and induced pluripotent stem cells (e.g., those from which lymphoid cells may be derived or differentiated). T cells can be lymphocytes that mature in the thymus and are chiefly responsible for cell-mediated immunity. T cells are involved in the adaptive immune system. In some embodiments, T cells of the present disclosure can be any type of T cells, including, without limitation, T helper cells, cytotoxic T cells, memory T cells (including central memory T cells, stem-cell-like memory T cells (or stem-like memory T cells), and two types of effector memory T cells: e.g., T_{EM} cells and T_{EMRA} cells, regulatory T cells (also known as suppressor T cells), natural killer T cells, mucosal associated invariant T cells, and γδ T cells. Cytotoxic T cells (CTL or killer T cells) are a subset of T lymphocytes capable of inducing the death of infected somatic or tumor cells. A patient's own T cells may be genetically modified to target specific antigens through the introduction of one or more chimeric receptors, such as a chimeric TCRs or CARs.

Natural killer (NK) cells can be lymphocytes that are part of cell-mediated immunity and act during the innate immune response. NK cells do not require prior activation in order to perform their cytotoxic effect on target cells.

In some embodiments, an immunoresponsive cell of the present disclosure is a T cell. T cells of the present disclosure may be autologous, allogeneic, or derived in vitro from engineered progenitor or stem cells.

In some embodiments, an immunoresponsive cell of the present disclosure is a universal T cell with deficient TCR-αβ. Methods of developing universal T cells are described in the art, for example, in Valton et al., Molecular Therapy (2015); 23 9, 1507-1518, and Torikai et al., Blood 2012 119:5697-5705.

In some embodiments, an immunoresponsive cell of the present disclosure is an isolated immunoresponsive cell comprising one or more chimeric receptors of the present disclosure. In some embodiments, the immunoresponsive cell comprises one or more, two or more, three or more, four or more, five or more, six or more, seven or more, eight or more, nine or more, or ten or more chimeric receptors of the present disclosure.

In some embodiments, an immunoresponsive cell is a T cell. In some embodiments, an immunoresponsive cell is a Natural Killer (NK) cell.

In some embodiments, an immunoresponsive cell expresses or is capable of expressing an immune receptor. Immune receptors generally are capable of inducing signal transduction or changes in protein expression in the immune receptor-expressing cell that results in the modulation of an immune response upon binding to a cognate ligand (e.g., regulate, activate, initiate, stimulate, increase, prevent, attenuate, inhibit, reduce, decrease, inhibit, or suppress an immune response). For example, when CD3 chains present in a TCR/CAR cluster in response to ligand binding, an immunoreceptor tyrosine-based activation motifs (ITAMs)-meditated signal transduction cascade is produced. Specifically, in certain embodiments, when an endogenous TCR, exogenous TCR, chimeric TCR, or a CAR (specifically an activating CAR) binds their respective antigen, a formation of an immunological synapse occurs that includes clustering of many molecules near the bound receptor (e.g. CD4 or CD8, CD3γ/δ/ε/ζ, etc.). This clustering of membrane bound signaling molecules allows for ITAM motifs contained within the CD3 chains to become phosphorylated that in turn can initiate a T cell activation pathway and ultimately activates transcription factors, such as NF-κB and AP-1. These transcription factors are capable of inducing global gene expression of the T cell to increase IL-2 production for proliferation and expression of master regulator T cell proteins in order to initiate a T cell mediated immune response, such as cytokine production and/or T cell mediated killing.

### Cells Expressing Multiple Chimeric Antigen Receptors

In some embodiments, a cell of the present disclosure (e.g., an immunoresponsive cell) comprises two or more chimeric receptors of the present disclosure. In some embodiments, the cell comprises two or more chimeric receptors, wherein a first of the two or more chimeric receptors is an activating chimeric receptor and a second of the two or more chimeric receptors is a chimeric inhibitory receptor. In some embodiments, the cell comprises a first activating chimeric receptor and a second activating chimeric receptor. In some embodiments, the cell comprises three or more chimeric receptors, wherein at least one of the three or more chimeric receptors is an activating chimeric receptor. In some embodiments, the cell comprises three or more chimeric receptors, wherein at least one of the three or more chimeric receptors is a chimeric inhibitory receptor. In some embodiments, the cell comprises four or more chimeric receptors. In some embodiments, the cell comprises five or more chimeric receptors.

In some embodiments, each of the two or more chimeric receptors comprise a different antigen-binding domain, e.g., that binds to the same antigen or to a different antigen. In some embodiments each antigen bound by the two or more chimeric receptors are expressed on the same cell, such as an epithelial cell type (e.g., same epithelial cell type).

In embodiments where a cell of the present disclosure (e.g., an immunoresponsive cell) expresses two or more distinct chimeric receptors, the antigen-binding domain of each of the different chimeric receptors may be designed such that the antigen-binding domains do not interact with one another. For example, a cell of the present disclosure (e.g., an immunoresponsive cell) expressing a first chimeric receptor and a second chimeric receptor may comprise a first chimeric receptor that comprises an antigen-binding domain that does not form an association with the antigen-binding domain of the second chimeric receptor. For example, the antigen-binding domain of the first chimeric receptor may comprise an antibody fragment, such as an scFv, while the antigen-binding domain of the second chimeric receptor may comprise a V_{H}H.

Without wishing to be bound by theory, it is believed that in cells having a plurality of chimeric membrane embedded receptors that each comprise an antigen-binding domain, interactions between the antigen-binding domains of each of the receptors can be undesirable, because such interactions may inhibit the ability of one or more of the antigen-binding domains to bind their cognate antigens. Accordingly, in embodiments where cells of the present disclosure (e.g., immunoresponsive cells) express two or more chimeric receptors, the chimeric receptors comprise antigen-binding domains that minimize such inhibitory interactions. In one embodiment, the antigen-binding domain of one chimeric receptor comprises an scFv and the antigen-binding domain of the second chimeric receptor comprises a single VH domain, e.g., a camelid, shark, or lamprey single VH domain, or a single VH domain derived from a human or mouse sequence.

In some embodiments, when present on the surface of a cell, binding of the antigen-binding domain of the first chimeric receptor to its cognate antigen is not substantially reduced by the presence of the second chimeric receptor. In some embodiments, binding of the antigen-binding domain of the first chimeric receptor to its cognate antigen in the presence of the second chimeric receptor is 85%, 90%, 95%, 96%, 97%, 98%, or 99% of binding of the antigen-binding domain of the first chimeric receptor to its cognate antigen in the absence of the second chimeric receptor. In some embodiments, when present on the surface of a cell, the antigen-binding domains of the first chimeric receptor and the second chimeric receptor associate with one another less than if both were scFv antigen-binding domains. In some embodiments, the antigen-binding domains of the first chimeric receptor and the second chimeric receptor associate with one another 85%, 90%, 95%, 96%, 97%, 98%, or 99% less than if both were scFv antigen-binding domains.

### Co-Stimulatory Ligands

In some embodiments, a cell of the present disclosure (e.g., an immunoresponsive cell) can further include one or more recombinant or exogenous co-stimulatory ligands. For example, the cell can be further transduced with one or more co-stimulatory ligands, such that the cell co-expresses or is induced to co-express one or more chimeric receptors and one or more co-stimulatory ligands. Without wishing to be bound by theory, it is believed that the interaction between the one or more chimeric receptors and the one or more co-stimulatory ligands may provide a non-antigen-specific signal important for full activation of the cell. Examples of suitable co-stimulatory ligands include, without limitation, members of the tumor necrosis factor (TNF) superfamily, and immunoglobulin (Ig) superfamily ligands. TNF is a cytokine involved in systemic inflammation and stimulates the acute phase reaction. Its primary role is in the regulation of immune cells. Members of TNF superfamily share a number of common features. The majority of TNF superfamily members are synthesized as type II transmembrane proteins (extracellular C-terminus) containing a short cytoplasmic segment and a relatively long extracellular region. Examples of suitable TNF superfamily members include, without limitation, nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin-alpha (LTa), lymphotoxin-beta (LTP), CD257/B cell-activating factor (B AFF)/Bly s/THANK/Tall-1, glucocorticoid-induced TNF Receptor ligand (GITRL), and TNF-related apoptosis-inducing ligand (TRAIL), LIGHT (TNFSF 14). The immunoglobulin (Ig) superfamily is a large group of cell surface and soluble proteins that are involved in the recognition, binding, or adhesion processes of cells. These proteins share structural features with immunoglobulins and possess an immunoglobulin domain (fold). Examples of suitable immunoglobulin superfamily ligands include, without limitation, CD80 and CD86, both ligands for CD28, PD-L1/(B7-H1) that are ligands for PD-1. In certain embodiments, the one or more co-stimulatory ligands are selected from 4-1BBL, CD80, CD86, CD70, OX40L, CD48, TNFRSF14, PD-L1, and combinations thereof.

In some embodiments, a cell of the present disclosure (e.g., an immunoresponsive cell) comprises one or more recombinant or exogenous co-stimulatory ligands regulated by an engineered CCL3-promoter described herein, e.g., an engineered CCL-3 promoter from Table 1. In certain embodiments, a TNF superfamily member (e.g., nerve growth factor (NGF), CD40L (CD40L)/CD 154, CD137L/4-1BBL, TNF-α, CD134L/OX40L/CD252, CD27L/CD70, Fas ligand (FasL), CD30L/CD153, tumor necrosis factor beta (TNFP)/lymphotoxin—alpha (LTa), lymphotoxin-beta (LTP), CD257/B cell-activating factor (B AFF)/Bly s/THANK/Tall-1, glucocorticoid-induced TNF Receptor ligand (GITRL), and TNF-related apoptosis-inducing ligand (TRAIL), LIGHT (TNFSF 14)) is regulated by an engineered CCL3-promoter described herein, e.g., an engineered CCL-3 promoter from Table 1. In certain embodiments, an Ig superfamily member ligand (e.g., CD80, CD86, PD-L1, and B7-H1) is regulated by an engineered CCL3-promoter described herein, e.g., an engineered CCL-3 promoter from Table 1. In certain embodiments, a co-stimulatory ligand (e.g., 4-1BBL, CD80, CD86, CD70, OX40L, CD48, TNFRSF14, PD-L1, and combinations thereof) is regulated by an engineered CCL-3 promoter described herein, e.g., an engineered CCL-3 promoter from Table 1.

### Chemokine Receptors

In some embodiments, a cell of the present disclosure (e.g., an immunoresponsive cell) comprises one or more chimeric receptors and may further include one or more chemokine receptors. For example, transgenic expression of chemokine receptor CCR2b or CXCR2 in cells, such as T cells, enhances trafficking to CCL2-secreting or CXCL1-secreting solid tumors (Craddock et al, J Immunother. 2010 October; 33(8):780-8 and Kershaw et al. Hum Gene Ther. 2002 Nov. 1; 13(16): 1971-80). Without wishing to be bound by theory, it is believed that chemokine receptors expressed on chimeric receptor-expressing cells of the present disclosure may recognize chemokines secreted by tumors and improve targeting of the cell to the tumor, which may facilitate the infiltration of the cell to the tumor and enhance the antitumor efficacy of the cell. Chemokine receptors of the present disclosure may include a naturally occurring chemokine receptor, a recombinant chemokine receptor, or a chemokine-binding fragment thereof. Examples of suitable chemokine receptors that may expressed on a cell of the present disclosure include, without limitation, a CXC chemokine receptor, such as CXCR1, CXCR2, CXCR3, CXCR4, CXCR5, CXCR6, or CXCR7; a CC chemokine receptor, such as CCR1, CCR2, CCR3, CCR4, CCR5, CCR6, CCR7, CCR8, CCR9, CCR10, or CCR11; a CX3C chemokine receptor, such as CX3CR1; an XC chemokine receptor, such as XCR1; and chemokine-binding fragments thereof. In some embodiments, the chemokine receptor to be expressed on the cell is chosen based on the chemokines secreted by the tumor.

In some embodiments, a cell of the present disclosure (e.g., an immunoresponsive cell) comprises one or more chemokine receptors regulated by an engineered CCL3-promoter described herein, e.g., an engineered CCL-3 promoter from Table 1. Examples of such chemokine receptors include, for example and without limitation, a CXC chemokine receptor, such as CXCR1, CXCR2, CXCR3, CXCR4, CXCR5, CXCR6, or CXCR7; a CC chemokine receptor, such as CCR1, CCR2, CCR3, CCR4, CCR5, CCR6, CCR7, CCR8, CCR9, CCR10, or CCR11; a CX3C chemokine receptor, such as CX3CR1; an XC chemokine receptor, such as XCR1; and chemokine-binding fragments thereof. In certain embodiments, the chemokine receptor regulated by the engineered CCL3-promoter described herein, e.g., an engineered CCL-3 promoter from Table 1, is chosen based on the chemokines secreted by the tumor.

### Chimeric Receptor Regulation

Some embodiments of the present disclosure relate to regulating one or more chimeric receptor activities of chimeric receptor-expressing cells of the present disclosure. There are several ways chimeric receptor activities can be regulated. In some embodiments, a regulatable chimeric receptor, wherein one or more chimeric receptor activities can be controlled, may be desirable to optimize the safety and/or efficacy of the chimeric receptor therapy. For example, inducing apoptosis using a caspase fused to a dimerization domain (see, e.g., Di et al., N Engl. J. Med. 2011 Nov. 3; 365(18): 1673-1683) can be used as a safety switch in the chimeric receptor therapy. In some embodiments, a chimeric receptor-expressing cell of the present disclosure can also express an inducible Caspase-9 (iCaspase-9) that, upon administration of a dimerizer drug, such as rimiducid (IUPAC name: [(1R)-3-(3,4-dimethoxyphenyl)-1-[3-[2-[2-[[2-[3-[(1R)-3-(3,4-dimethoxyphenyl)-1-[(2S)-1-[(2S)-2-(3,4,5-trimethoxyphenyl)butanoyl]piperidine-2-carbonyl]oxypropyl]phenoxy]acetyl]amino]ethylamino]-2-oxoethoxy]phenyl]propyl](2S)-1-[(2S)-2-(3,4,5-trimethoxyphenyl)butanoyl]piperidine-2-carboxylate), induces activation of the Caspase-9 and results in apoptosis of the cells. In some embodiments, the iCaspase-9 contains a binding domain that comprises a chemical inducer of dimerization (CID) that mediates dimerization in the presence of the CID, which results in inducible and selective depletion of the chimeric receptor-expressing cells.

Alternatively, in some embodiments a chimeric receptor of the present disclosure may be regulated by utilizing a small molecule or an antibody that deactivates or otherwise inhibits chimeric receptor activity. For example, an antibody may delete the chimeric receptor-expressing cells by inducing antibody dependent cell-mediated cytotoxicity (ADCC). In some embodiments, a chimeric receptor-expressing cell of the present disclosure may further express an antigen that is recognized by a molecule that is capable of inducing cell death by ADCC or complement-induced cell death. For example, a chimeric receptor-expressing cell of the present disclosure may further express a receptor capable of being targeted by an antibody or antibody fragment. Examples of suitable receptors that may be targeted by an antibody or antibody fragment include, without limitation, EpCAM, VEGFR, integrins (e.g., ανβ3, α4, αI/¾β3, α4β7, α5β1, ανβ3, αν), members of the TNF receptor superfamily (e.g., TRAIL-R1 and TRAIL-R2), PDGF receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11, CD11a/LFA-1, CD15, CD18/ITGB2, CD19, CD20, CD22, CD23/IgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof.

In some embodiments, a chimeric receptor-expressing cell of the present disclosure may also express a truncated epidermal growth factor receptor (EGFR) that lacks signaling capacity but retains an epitope that is recognized by molecules capable of inducing ADCC (e.g., WO2011/056894).

In some embodiments, a chimeric receptor-expressing cell of the present disclosure further includes a highly expressing compact marker/suicide gene that combines target epitopes from both CD32 and CD20 antigens in the chimeric receptor-expressing cell, which binds an anti-CD20 antibody (e.g., rituximab) resulting in selective depletion of the chimeric receptor-expressing cell by ADCC. Other methods for depleting chimeric receptor-expressing cells of the present disclosure my include, without limitation, administration of a monoclonal anti-CD52 antibody that selectively binds and targets the chimeric receptor-expressing cell for destruction by inducing ADCC. In some embodiments, the chimeric receptor-expressing cell can be selectively targeted using a chimeric receptor ligand, such as an anti-idiotypic antibody. In some embodiments, the anti-idiotypic antibody can cause effector cell activity, such as ADCC or ADC activity. In some embodiments, the chimeric receptor ligand can be further coupled to an agent that induces cell killing, such as a toxin. In some embodiments, a chimeric receptor-expressing cell of the present disclosure may further express a target protein recognized by a cell depleting agent of the present disclosure. In some embodiments, the target protein is CD20 and the cell depleting agent is an anti-CD20 antibody. In such embodiments, the cell depleting agent is administered once it is desirable to reduce or eliminate the chimeric receptor-expressing cell. In some embodiments, the cell depleting agent is an anti-CD52 antibody.

In some embodiments, a regulated chimeric receptor comprises a set of polypeptides, in which the components of a chimeric receptor of the present disclosure are partitioned on separate polypeptides or members. For example, the set of polypeptides may include a dimerization switch that, when in the presence of a dimerization molecule, can couple the polypeptides to one another to form a functional chimeric receptor.

### Chimeric Receptor-Encoding Polynucleotide Constructs

Certain aspects of the present disclosure relate to polynucleotides (e.g., isolated polynucleotides) encoding one or more chimeric receptors of the present disclosure. In some embodiments, the polynucleotide is an RNA construct, such as a messenger RNA (mRNA) transcript or a modified RNA. In some embodiments, the polynucleotide is a DNA construct.

In some embodiments, a polynucleotide of the present disclosure encodes a chimeric receptor that comprises one or more antigen-binding domain, where each domain binds to a target antigen, a transmembrane domain, and one or more intracellular signaling domains. In some embodiments, the polynucleotide encodes a chimeric receptor that comprises an antigen-binding domain, a transmembrane domain, a primary signaling domain (e.g., CD3-zeta domain), and one or more costimulatory signaling domains. In some embodiments, the polynucleotide further comprises a nucleic acid sequence encoding a spacer region. In some embodiments, the antigen-binding domain is connected to the transmembrane domain by the spacer region. In some embodiments, the nucleic acid further comprises a nucleotide sequence encoding a leader sequence.

The polynucleotides of the present disclosure may be obtained using any suitable recombinant methods known in the art, including, without limitation, by screening libraries from cells expressing the gene of interest, by deriving the gene of interest from a vector known to include the gene, or by isolating the gene of interest directly from cells and tissues containing the gene using standard techniques. Alternatively, the gene of interest may be produced synthetically.

In some embodiments, a polynucleotide of the present disclosure in comprised within a vector. In some embodiments, a polynucleotide of the present disclosure is expressed in a cell via transposons, a CRISPR/Cas9 system, a TALEN, or a zinc finger nuclease.

In some embodiments, expression of a polynucleotide encoding a chimeric receptor of the present disclosure may be achieved by operably linking the nucleic acid to a promoter and incorporating the construct into an expression vector. A suitable vector can replicate and integrate in eukaryotic cells. Typical cloning vectors contain transcription and translation terminators, initiation sequences, and promoters useful for regulating expression of the desired nucleic acid.

In some embodiments, expression constructs of the present disclosure may also be used for nucleic acid immunization and gene therapy, using standard gene delivery protocols (e.g., U.S. Pat. Nos. 5,399,346, 5,580,859, and 5,589,466). In some embodiments, a vector of the present disclosure is a gene therapy vector.

A polynucleotide of the present disclosure can be cloned into a number of types of vectors. For example, the polynucleotide can be cloned into a vector including, without limitation, a plasmid, a phagemid, a phage derivative, an animal virus, or a cosmid. In some embodiments, the vector may be an expression vector, a replication vector, a probe generation vector, or a sequencing vector.

In some embodiments, the plasmid vector comprises a transposon/transposase system to incorporate the polynucleotides of the present disclosure into the host cell genome. Methods of expressing proteins in immune cells using a transposon and transposase plasmid system are generally described in Chicaybam L, Hum Gene Ther. 2019 April; 30(4):511-522. doi: 10.1089/hum.2018.218; and P\acute{t}ac̆ková P, Cytotherapy. 2018 April; 20(4):507-520. doi: 10.1016/j.jcyt.2017.10.001, each of which is hereby incorporated by reference in their entirety. In some embodiments, the transposon system is the Sleeping Beauty transposon/transposase or the piggyBac transposon/transposase.

In some embodiments, an expression vector of the present disclosure may be provided to a cell in the form of a viral vector. Suitable viral vector systems are well known in the art. For example, viral vectors may be derived from retroviruses, adenoviruses, adeno-associated viruses, herpes viruses, and lentiviruses. In some embodiments, a vector of the present disclosure is a lentiviral vector. Lentiviral vectors are suitable for long-term gene transfer as such vectors allow long-term, stable integration of a transgene and its propagation in daughter cells. Lentiviral vectors are also advantageous over vectors derived from onco-retroviruses (e.g., murine leukemia viruses) in that lentiviral vectors can transduce non-proliferating cells. In some embodiments, a vector of the present disclosure is an adenoviral vector (A5/35). In some embodiments, a vector of the present disclosure contains an origin of replication functional in at least one organism, a promoter sequence, convenient restriction endonuclease sites, and one or more selectable markers (e.g., WO01/96584; WO01/29058; and U.S. Pat. No. 6,326,193). A number of viral based systems have been developed for gene transfer into mammalian cells. A selected gene can be inserted into a vector and packaged in retroviral particles using techniques known in the art. The recombinant virus can then be isolated and delivered to mammalian cells either in vivo or ex vivo. A number of retroviral systems are known in the art.

In some embodiments, vectors of the present disclosure include additional promoter elements, such as enhancers that regulate the frequency of transcriptional initiation. Enhancers are typically located in a region that is 30 bp to 110 bp upstream of the start site, although a number of promoters have been shown to contain functional elements downstream of the start site as well. The spacing between promoter elements may be flexible, so that promoter function is preserved when elements are inverted or moved relative to one another. For example, in the thymidine kinase (tk) promoter the spacing between promoter elements can be increased to 50 bp apart before activity begins to decline. Depending on the promoter, individual elements may function either cooperatively or independently to activate transcription. Exemplary promoters may include, without limitation, the SFFV gene promoter, the EFS gene promoter, the CMV IE gene promoter, the EFla promoter, the ubiquitin C promoter, and the phosphoglycerokinase (PGK) promoter.

In some embodiments, a promoter that is capable of expressing a polynucleotide of the present disclosure in a mammalian cell, such as an immunoresponsive cell of the present disclosure, is the EFla promoter. The native EFla promoter drives expression of the alpha subunit of the elongation factor-1 complex, which is responsible for the enzymatic delivery of aminoacyl tRNAs to the ribosome. The EFla promoter has been widely used in mammalian expression plasmids and has been shown to be effective in driving chimeric receptor expression from polynucleotide cloned into a lentiviral vector.

In some embodiments, a promoter that is capable of expressing a polynucleotide of the present disclosure in a mammalian cell, such as an immunoresponsive cell of the present disclosure, is a constitutive promoter. For example, a suitable constitutive promoter is the spleen focus forming virus (SFFV) promoter. Another example of a suitable constitutive promoter is the immediate early cytomegalovirus (CMV) promoter. The CMV promoter is a strong constitutive promoter that is capable of driving high levels of expression of any polynucleotide sequence operatively linked to the promoter. Other suitable constitutive promoters include, without limitation, a ubiquitin C (UbiC) promoter, a simian virus 40 (SV40) early promoter, a mouse mammary tumor virus (MMTV) promoter, a human immunodeficiency virus (HIV) long terminal repeat (LTR) promoter, a MoMuLV promoter, an avian leukemia virus promoter, an Epstein-Barr virus immediate early promoter, a Rous sarcoma virus promoter, an actin promoter, a myosin promoter, an elongation factor-1a promoter, a hemoglobin promoter, and a creatine kinase promoter.

In some embodiments, a promoter that is capable of expressing a polynucleotide of the present disclosure in a mammalian cell, such as an immunoresponsive cell of the present disclosure, is an inducible promoter. Use of an inducible promoter may provide a molecular switch that is capable of inducing or repressing expression of a polynucleotide of the present disclosure when the promoter is operatively linked to the polynucleotide. Examples of inducible promoters include, without limitation, a metallothionine promoter, a glucocorticoid promoter, a progesterone promoter, and a tetracycline promoter.

In some embodiments, a vector of the present disclosure may further comprise a signal sequence to facilitate secretion, a polyadenylation signal and transcription terminator, an element allowing episomal replication, and/or elements allowing for selection.

In some embodiments, a vector of the present disclosure can further comprise a selectable marker gene and/or reporter gene to facilitate identification and selection of chimeric receptor-expressing cells from a population of cells that have been transduced with the vector. In some embodiments, the selectable marker may be encoded by a polynucleotide that is separate from the vector and used in a co-transfection procedure. Either selectable marker or reporter gene may be flanked with appropriate regulator sequences to allow expression in host cells. Examples of selectable markers include, without limitation, antibiotic-resistance genes, such as neo and the like.

In some embodiments, reporter genes may be used for identifying transduced cells and for evaluating the functionality of regulatory sequences. As disclosed herein, a reporter gene is a gene that is not present in or expressed by the recipient organism or tissue and that encodes a polypeptide whose expression results in an easily detectable property, such as enzymatic activity. Expression of the reporter gene can be assayed at a suitable time after the polynucleotide has been introduced into the recipient cells. Examples of reporter genes include, without limitation, genes encoding for luciferase, genes encoding for beta-galactosidase, genes encoding for chloramphenicol acetyl transferase, genes encoding for secreted alkaline phosphatase, and genes encoding for green fluorescent protein. Suitable expression systems are well known in the art and may be prepared using known techniques or obtained commercially. In some embodiments, a construct with a minimal 5′ flanking region showing the highest level of expression of the reporter gene is identified as the promoter. Such promoter regions may be linked to a reporter gene and used to evaluate agents for the ability to modulate promoter-driven transcription.

In some embodiments, a vector comprising a polynucleotide sequence encoding a chimeric receptor of the present disclosure further comprises a second polynucleotide encoding a polypeptide that increases the activity of the chimeric receptor.

In embodiments where a chimeric receptor-expressing cell comprises two or more chimeric receptors, a single polynucleotide may encode the two or more chimeric receptors under a single regulatory control element (e.g., promoter) or under separate regulatory control elements for each chimeric receptor-encoding nucleotide sequence comprised in the polynucleotide. In some embodiments where a chimeric receptor-expressing cell comprises two or more chimeric receptors, each chimeric receptor may be encoded by a separate polynucleotide. In some embodiments, each separate polynucleotide comprises its own control element (e.g., promoter). In some embodiments, a single polynucleotide encodes the two or more chimeric receptors and the chimeric receptor-encoding nucleotide sequences are in the same reading frame and are expressed as a single polypeptide chain. In such embodiments, the two or more chimeric receptors may be separated by one or more peptide cleavage sites, such as auto-cleavage sites or substrates for an intracellular protease. Suitable peptide cleavage sites may include, without limitation, a T2A peptide cleavage site, a P2A peptide cleavage site, an E2A peptide cleavage sire, and an F2A peptide cleavage site. In some embodiments, the two or more chimeric receptors comprise a T2A peptide cleavage site. In some embodiments, the two or more chimeric receptors comprise an E2A peptide cleavage site. In some embodiments, the two or more chimeric receptors comprise a T2A and an E2A peptide cleavage site.

Methods of introducing and expressing genes into a cell are well known in the art. For example, in some embodiments, an expression vector can be transferred into a host cell by physical, chemical, or biological means. Examples of physical means for introducing a polynucleotide into a host cell include, without limitation, calcium phosphate precipitation, lipofection, particle bombardment, microinjection, and electroporation. Examples of chemical means for introducing a polynucleotide into a host cell include, without limitation, colloidal dispersion systems, macromolecule complexes, nanocapsules, microspheres, beads, and lipid-based systems including oil-in-water emulsions, micelles, mixed micelles, and liposomes. Examples of biological means for introducing a polynucleotide into a host cell include, without limitation, the use of DNA and RNA vectors.

In some embodiments, liposomes may be used as a non-viral delivery system to introduce a polynucleotide or vector of the present disclosure into a host cell in vitro, ex vivo, or in vivo. In some embodiments, the polynucleotide may be associated with a lipid, for example by being encapsulated in the aqueous interior of a liposome, being interspersed within the lipid bilayer of a liposome, being attached to a liposome via a linking molecule that is associated with both the liposome and the polynucleotide, being entrapped in a liposome, being complexed with a liposome, being dispersed in a solution containing a lipid, being mixed with a lipid, being combined with a lipid, being contained as a suspension in a lipid, being contained or complexed with a micelle, or otherwise being associated with a lipid. As disclosed herein, lipid-associated polynucleotide or vector compositions are not limited to any particular structure in solution. In some embodiments, such compositions may be present in a bilayer structure, as micelles or with a “collapsed” structure. Such compositions may also be interspersed in a solution, forming aggregates that are not uniform in size or shape. As disclosed herein, lipids are fatty substances that may be naturally occurring or synthetic. In some embodiments, lipids can include the fatty droplets that naturally occur in the cytoplasm or the class of compounds that contain long-chain aliphatic hydrocarbons and their derivatives, such as fatty acids, alcohols, amines, amino alcohols, and aldehydes. Suitable lipids may be obtained from commercial sources and include, without limitation, dimyristyl phosphatidylcholine (“DMPC”), dicetylphosphate (“DCP”), cholesterol, and dimyristylphosphatidylglycerol (“DMPG”). Stock solutions of lipids in chloroform or chloroform/methanol can be stored at about −20° C. Chloroform is used as the solvent, as it is more readily evaporated than methanol. As used herein, a “liposome” may encompass a variety of single and multilamellar lipid vehicles formed by the generation of enclosed lipid bilayers or aggregates. In some embodiments, liposomes can be characterized as having vesicular structures with a phospholipid bilayer membrane and an inner aqueous medium. In some embodiments, multilamellar liposomes may have multiple lipid layers separated by aqueous medium. Multilamellar liposomes can form spontaneously when phospholipids are suspended in an excess of aqueous solution. In some embodiments, lipid components may undergo self-rearrangement before the formation of closed structures and can entrap water and dissolved solutes between the lipid bilayers. In some embodiments, the lipids may assume a micellar structure or merely exist as nonuniform aggregates of lipid molecules.

In some embodiments, a polynucleotide or vector of the present disclosure is introduced into a mammalian host cell, such as an immunoresponsive cell of the present disclosure. In some embodiments, the presence of a polynucleotide or vector of the present disclosure in a host cell may be confirmed by any suitable assay known in the art, including without limitation Southern blot assays, Northern blot assays, RT-PCR, PCR, ELISA assays, and Western blot assays.

In some embodiments, a polynucleotide or vector of the present disclosure is stably transduced into an immunoresponsive cell of the present disclosure. In some embodiments, cells that exhibit stable expression of the polynucleotide or vector express the encoded chimeric receptor for at least 1 week, at least 2 weeks, at least 3 weeks, at least 4 weeks, at least 5 weeks, at least 6 weeks, at least 7 weeks, at least 8 weeks, at least 3 months, at least 6 months, at least 9 months, or at least 12 months after transduction.

In embodiments where a chimeric receptor of the present disclosure is transiently expressed in a cell, a chimeric receptor-encoding polynucleotide or vector of the present disclosure is transfected into an immunoresponsive cell of the present disclosure. In some embodiments the immunoresponsive cell expresses the chimeric receptor for about 4 days, about 5 days, about 6 days, about 7 days, about 8 days, about 9 days, about 10 days, about 11 days, about 12 days, about 13 days, about 14 days, or about 15 days after transfection.

### Methods of Engineering Cells

Also provided herein are compositions and methods for engineering cells to produce the activation conditional control polypeptide (ACP) and one or more effectors molecules encoded by any engineered nucleic acid comprising the first and second expression cassettes as described herein or otherwise known in the art.

In general, cells are engineered to produce ACPs and effector molecules through introduction (i.e., delivery) of one or more polynucleotides of the present disclosure comprising the first promoter and the exogenous polynucleotide sequence encoding the ACP and the second expression cassette comprising an ACP-responsive promoter and the second exogenous sequence encoding one or more effector molecules into the cell's cytosol and/or nucleus. For example, the polynucleotide expression cassettes encoding the ACP polypeptide and the one or more effector molecules can be any of the engineered nucleic acids described herein. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

In some embodiments, the engineered cell is transduced using an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. In some embodiments, the oncolytic virus is a recombinant oncolytic virus comprising the first expression cassette and the second expression cassette. In some embodiments, the oncolytic virus further comprises the third expression cassette.

The virus, including any of the oncolytic viruses described herein, can be a recombinant virus that encodes one more transgenes encoding one or more effector molecules, such as any of the engineered nucleic acids described herein. The virus, including any of the oncolytic viruses described herein, can be a recombinant virus that encodes one more transgenes encoding one or more of the two or more effector molecules, such as any of the engineered nucleic acids described herein. In some embodiments, the cell is engineered via transduction with an oncolytic virus.

### Viral-Mediated Delivery

Viral vector-based delivery platforms can be used to engineer cells. In general, a viral vector-based delivery platform engineers a cell through introducing (i.e., delivering) into a host cell. For example, a viral vector-based delivery platform can engineer a cell through introducing any of the engineered nucleic acids described herein. A viral vector-based delivery platform can be a nucleic acid, and as such, an engineered nucleic acid can also encompass an engineered virally-derived nucleic acid. Such engineered virally-derived nucleic acids can also be referred to as recombinant viruses or engineered viruses.

A viral vector-based delivery platform can encode more than one engineered nucleic acid, gene, or transgene within the same nucleic acid. For example, an engineered virally-derived nucleic acid, e.g., a recombinant virus or an engineered virus, can encode one or more transgenes, including, but not limited to, any of the engineered nucleic acids described herein that encode one or more effector molecules. The one or more transgenes encoding the one or more effector molecules can be configured to express the one or more effector molecules. A viral vector-based delivery platform can encode one or more genes in addition to the one or more transgenes (e.g., transgenes encoding the one or more effector molecules), such as viral genes needed for viral infectivity and/or viral production (e.g., capsid proteins, envelope proteins, viral polymerases, viral transcriptases, etc.), referred to as cis-acting elements or genes.

A viral vector-based delivery platform can comprise more than one viral vector, such as separate viral vectors encoding the engineered nucleic acids, genes, or transgenes described herein, and referred to as trans-acting elements or genes. For example, a helper-dependent viral vector-based delivery platform can provide additional genes needed for viral infectivity and/or viral production on one or more additional separate vectors in addition to the vector encoding the one or more effector molecules. One viral vector can deliver more than one engineered nucleic acids, such as one vector that delivers engineered nucleic acids that are configured to produce two or more effector molecules. More than one viral vector can deliver more than one engineered nucleic acids, such as more than one vector that delivers one or more engineered nucleic acid configured to produce one or more effector molecules. The number of viral vectors used can depend on the packaging capacity of the above-mentioned viral vector-based vaccine platforms, and one skilled in the art can select the appropriate number of viral vectors.

In general, any of the viral vector-based systems can be used for the in vitro production of molecules, such as effector molecules, or used in vivo and ex vivo gene therapy procedures, e.g., for in vivo delivery of the engineered nucleic acids encoding one or more effector molecules. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, a adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e. delivery) into a host cell, infected cells (i.e., an engineered cell) can express, and in some case secrete, the one or more effector molecules. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms can be a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., an engineered nucleic acid) encoding one or more effector molecules. The transgenes encoding the one or more effector molecules can be configured to express the one or more effector molecules.

In some embodiments, the virus is selected from: a lentivirus, a retrovirus, an oncolytic virus, an adenovirus, an adeno-associated virus (AAV), and a virus-like particle (VLP).

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids (e.g., transgenes encoding the one or more effector molecules) into the target cell to provide permanent transgene expression. Retroviral-based delivery systems include, but are not limited to, those based upon murine leukemia, virus (MuLV), gibbon ape leukemia virus (GaLV), Simian Immuno deficiency vims (SIV), human immuno deficiency vims (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of effector molecules, or used in vivo and ex vivo gene therapy procedures, e.g., for in vivo delivery of the engineered nucleic acids encoding one or more effector molecules (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78(12):6381-6388 (June 2004); Gao, et al, Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat &amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

### Lipid Structure Delivery Systems

Engineered nucleic acids of the present disclosure (e.g., any of the engineered nucleic acids described herein) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Felgner et al., Proc. Natl. Acad. Sci. USA 84: 7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of an engineered nucleic acid and/or viral delivery system can be used to avoid degradation, while also avoiding potential off-target affects. In certain examples, an engineered nucleic acid and/or viral delivery system is fully encapsulated within the delivery vehicle, such as within the aqueous interior of an LNP. Encapsulation of an engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with an engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., an engineered nucleic acid and/or viral delivery system) can be further treated or engineered to prepare them for administration.

### Nanoparticle Delivery

Nanomaterials can be used to deliver engineered nucleic acids (e.g., any of the engineered nucleic acids described herein). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery—A Review. Nanomaterials 2017, 7(5), 94), herein incorporated by reference for all purposes.

### Genomic Editing Systems

A genomic editing systems can be used to engineer a host genome to encode an engineered nucleic acid, such as an engineered nucleic acid of the present disclosure. In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate an engineered nucleic acid, such as an engineered nucleic acid of the present disclosure, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tc 1/mariner transposon superfamily, such as a Sleeping Beauty transposon system, described in more detail in Hudecek et al. (Crit Rev Biochem Mol Biol. 2017 August; 52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode an engineered nucleic acid, such as an engineered nucleic acid of the present disclosure. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids encoding one or more effector molecules).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid, such as an engineered nucleic acid encoding one or more of the effector molecules described herein. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and a RNA(s) that directs cleavage to a particular target sequence. An exemplary CRISPR-mediated gene editing system is the CRISPR/Cas9 systems comprised of a Cas9 nuclease and a RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can be also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The engineered cells described herein can be engineered using non-viral methods, e.g., the nuclease and/or CRISPR mediated gene editing systems described herein can be delivered to a cell using non-viral methods. The engineered cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA-binding domain of TALE (transcription activator-like effectors) and the catalytic domain of restriction endonuclease Fokl. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

### Other Engineering Delivery Systems

Various additional means to introduce engineered nucleic acids (e.g., any of the engineered nucleic acids described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., any of the engineered nucleic acids described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 Apr. 10; 27(4): 710-728) and Kaczmarek et al. (Genome Med. 2017; 9: 60), each herein incorporated by reference for all purposes.

### Methods of Use

Methods for treatment of diseases are also encompassed by this disclosure. Said methods include administering a therapeutically effective amount of an engineered nucleic acid, engineered cell, or isolated cell as described above. In some aspects, provided herein are methods of treating a subject in need thereof, the method comprising administering a therapeutically effective dose of any of the engineered cells, isolated cells, or compositions disclosed herein.

In some aspects, provided herein is a method of increasing expression of a target gene, e.g. a tumor suppressor gene.

In some aspects, provided herein a methods of increasing expression of a target gene, e.g. an immunomodulatory gene. Exemplary immunomodulatory genes include, for example and without limitation, cytokines, chemokines, receptors thereof, and derivatives thereof.

In some aspects, provided herein are methods of stimulating a cell-mediated immune response to a tumor cell in a subject, the method comprising administering to a subject having a tumor a therapeutically effective dose of any of the engineered cells, isolated cells, or compositions disclosed herein.

In some aspects, provided herein are methods of providing an anti-tumor immunity in a subject, the method comprising administering to a subject in need thereof a therapeutically effective dose of any of the engineered cells, isolated cells, or compositions disclosed herein.

In some aspects, provided herein are methods of treating a subject having cancer, the method comprising administering a therapeutically effective dose of any of the engineered cells, isolated cells, or compositions disclosed herein.

In some aspects, provided herein are methods of reducing tumor volume in a subject, the method comprising administering to a subject having a tumor a composition comprising any of the engineered cells, isolated cells, or compositions disclosed herein.

In some embodiments, the administering comprises systemic administration. In some embodiments, the administering comprises intratumoral administration. In some embodiments, the isolated cell is derived from the subject. In some embodiments, the isolated cell is allogeneic with reference to the subject.

In some embodiments, the method further comprises administering a checkpoint inhibitor. the checkpoint inhibitor is selected from: an anti-PD-1 antibody, an anti-PD-L1 antibody, an anti-PD-L2 antibody, an anti-CTLA-4 antibody, an anti-LAG-3 antibody, an anti-TIM-3 antibody, an anti-TIGIT antibody, an anti-VISTA antibody, an anti-KIR antibody, an anti-B7-H3 antibody, an anti-B7-H4 antibody, an anti-HVEM antibody, an anti-BTLA antibody, an anti-GAL9 antibody, an anti-A2AR antibody, an anti-phosphatidylserine antibody, an anti-CD27 antibody, an anti-TNFa antibody, an anti-TREM1 antibody, and an anti-TREM2 antibody. In some embodiments, the method further comprises administering an anti-CD40 antibody.

In some embodiments, the tumor is selected from: an adenocarcinoma, a bladder tumor, a brain tumor, a breast tumor, a cervical tumor, a colorectal tumor, an esophageal tumor, a glioma, a kidney tumor, a liver tumor, a lung tumor, a melanoma, a mesothelioma, an ovarian tumor, a pancreatic tumor, a gastric tumor, a testicular yolk sac tumor, a prostate tumor, a skin tumor, a thyroid tumor, and a uterine tumor.

Some methods comprise selecting a subject (or patient population) having a tumor (or cancer) and treating that subject with engineered cells or delivery vehicles that modulate tumor-mediated immunosuppressive mechanisms.

The methods provided herein also include delivering a preparation of engineered cells or delivery vehicles. A preparation, in some embodiments, is a substantially pure preparation, containing, for example, less than 5% (e.g., less than 4%, 3%, 2%, or 1%) of cells other than engineered cells. A preparation may comprise 1×10⁵ cells/kg to 1×10⁷ cells/kg cells.

The methods provided herein also include administering a drug or pharmaceutical composition in combination with a therapeutically effective dose of any of the engineered cells, isolated cells, or compositions disclosed herein such that the ACP is induced and/or that a repressible protease is repressed. For example, tamoxifen or a metabolite thereof (e.g., 4-hydroxytamoxifen, N-desmethyltamoxifen, tamoxifen-N-oxide, or endoxifen) can be administered to induce the ACP. The drug or pharmaceutical can be administered prior to, concurrently with, simultaneously with, and/or subsequent to administration of any of the engineered cells, isolated cells, or compositions disclosed herein. The drug or pharmaceutical can be administered serially. The drug or pharmaceutical can be administered concurrently or simultaneously with administration of any of the engineered cells, isolated cells, or compositions disclosed herein. The drug or pharmaceutical can be administered at separate intervals than (e.g., prior to or subsequent to) administration of any of the engineered cells, isolated cells, or compositions disclosed herein. The drug or pharmaceutical can be administered both concurrently/simultaneously as well as at separate intervals than any of the engineered cells, isolated cells, or compositions disclosed herein. The drug or pharmaceutical composition and the engineered cells, isolated cells, or compositions can be administered via different routes, e.g., the drug or pharmaceutical composition can be administered orally and the engineered cells, isolated cells, or compositions can be administered intraperitoneally, intravenously, subcutaneously, or any other route appropriate for administration, as will be appreciated by one skilled in the art.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The methods provided herein include administering a protease inhibitor. In some embodiments, the NS3 protease can be repressed by a protease inhibitor. Any suitable protease inhibitor can be used, including, but not limited to, simeprevir, danoprevir, asunaprevir, ciluprevir, boceprevir, sovaprevir, paritaprevir, telaprevir, grazoprevir, glecaprevir, and voxiloprevir, or any combination thereof. In some embodiments, the protease inhibitor is selected from: simeprevir, danoprevir, asunaprevir, ciluprevir, boceprevir, sovaprevir, paritaprevir, telaprevir, grazoprevir, glecaprevir, and voxiloprevir.

In some embodiments, the protease inhibitor is grazoprevir. In some embodiments, the protease inhibitor is a combination of grazoprevir and elbasvir (a NS5A inhibitor of the hepatitis C virus NS5A replication complex). Grazoprevir and elbasvir can be co-formulated as a pharmaceutical composition, such as in tablet form (e.g., the tablet available under the tradename Zepatier®). Grazoprevir and elbasvir can be co-formulated at a 2:1 weight ratio, respectively, such as at a unit dose of 100 mg grazoprevir 50 mg elbasvir (e.g., as in the tablet available under the tradename Zepatier®). The protease inhibitor can be administered at a dose capable of repressing a repressible protease domain of an ACP. The protease inhibitor can be administered at an approved dose for another indication. As an illustrative non-limiting example, Zepatier can be administered at its approved dose for treatment of HCV.

Grazoprevir, including in combination with elbasvir, can be administered orally in a dosage range of 0.001 to 1000 mg/kg of mammal (e.g., human) body weight per day in a single dose or in divided doses. One dosage range is 0.01 to 500 mg/kg body weight per day orally in a single dose or in divided doses. Another dosage range is 0.1 to 100 mg/kg body weight per day orally in single or divided doses. For oral administration, grazoprevir, including in combination with elbasvir, can be provided in the form of tablets or capsules containing 1.0 to 500 mg of the active ingredient, particularly 1, 5, 10, 15, 20, 25, 50, 75, 100, 150, 200, 250, 300, 400, 500, and 750 mg of the active ingredient for the symptomatic adjustment of the dosage to the patient to be treated. Generally, a total daily dosage of grazoprevir, including in combination with elbasvir, can range from about 1 to about 2500 mg per day, although variations will necessarily occur depending on the target of therapy, the patient and the route of administration. In one embodiment, the dosage of grazoprevir, including in combination with elbasvir, is from about 10 to about 1000 mg/day, administered in a single dose or in 2-4 divided doses. In another embodiment, the dosage of grazoprevir, including in combination with elbasvir, is from about 1 to about 500 mg/day, administered in a single dose or in 2-4 divided doses. In still another embodiment, the dosage of grazoprevir, including in combination with elbasvir, is from about 1 to about 100 mg/day, administered in a single dose or in 2-4 divided doses. In yet another embodiment, the dosage of grazoprevir, including in combination with elbasvir, is from about 1 to about 50 mg/day, administered in a single dose or in 2-4 divided doses. In another embodiment, the dosage of grazoprevir, including in combination with elbasvir, is from about 500 to about 1500 mg/day, administered in a single dose or in 2-4 divided doses. In still another embodiment, the dosage of grazoprevir, including in combination with elbasvir, is from about 500 to about 1000 mg/day, administered in a single dose or in 2-4 divided doses. In yet another embodiment, the dosage of grazoprevir, including in combination with elbasvir, is from about 100 to about 500 mg/day, administered in a single dose or in 2-4 divided doses.

### In Vivo Expression

The methods provided herein also include delivering a composition in vivo capable of producing the engineered cells described herein, e.g., capable of delivering any of the engineered nucleic acids described herein to a cell in vivo. Such compositions include any of the viral-mediated delivery platforms, any of the lipid structure delivery systems, any of the nanoparticle delivery systems, any of the genomic editing systems, or any of the other engineering delivery systems described herein capable of engineering a cell in vivo.

The methods provided herein also include delivering a composition in vivo capable of producing any of the effector molecules described herein. The methods provided herein also include delivering a composition in vivo capable of producing two or more of the effector molecules described herein. Compositions capable of in vivo production of effector molecules include, but are not limited to, any of the engineered nucleic acids described herein. Compositions capable of in vivo production of effector molecules can be a naked mRNA or a naked plasmid.

### Pharmaceutical Compositions

The engineered nucleic acid or engineered cell can be formulated in pharmaceutical compositions. These compositions can comprise, in addition to one or more of the engineered nucleic acids or engineered cells, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

Whether it is a polypeptide, nucleic acid, small molecule or other pharmaceutically useful compound according to the present disclosure that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Kits

Certain aspects of the present disclosure relate to kits for the treatment and/or prevention of a cancer (e.g., solid tumors). In certain embodiments, the kit includes a therapeutic or prophylactic composition comprising an effective amount of one or more chimeric receptors of the present disclosure, isolated nucleic acids of the present disclosure, vectors of the present disclosure, and/or cells of the present disclosure (e.g., immunoresponsive cells). In some embodiments, the kit comprises a sterile container. In some embodiments, such containers can be boxes, ampules, bottles, vials, tubes, bags, pouches, blister-packs, or other suitable container forms known in the art. The container may be made of plastic, glass, laminated paper, metal foil, or other materials suitable for holding medicaments.

In some embodiments, therapeutic or prophylactic composition is provided together with instructions for administering the therapeutic or prophylactic composition to a subject having or at risk of developing cancer (e.g., a solid tumor). In some embodiments, the instructions may include information about the use of the composition for the treatment and/or prevention of the disorder. In some embodiments, the instructions include, without limitation, a description of the therapeutic or prophylactic composition, a dosage schedule, an administration schedule for treatment or prevention of the disorder or a symptom thereof, precautions, warnings, indications, counter-indications, over-dosage information, adverse reactions, animal pharmacology, clinical studies, and/or references. In some embodiments, the instructions can be printed directly on the container (when present), or as a label applied to the container, or as a separate sheet, pamphlet, card, or folder supplied in or with the container.

### Additional Embodiments

Embodiment 1: An engineered CCL3 promoter comprising an ablation of at least one nucleotide motif, wherein the ablation increases inducibility of the engineered CCL3 promoter in the presence of an immune cell activation signal, as compared to inducibility of a wild-type CCL3 promoter in the presence of the same immune cell activation signal.
Embodiment 2: The engineered CCL3 promoter according to embodiment 1, wherein the wild-type CCL3 promoter comprises the nucleotide sequence of SEQ ID NO: 132.
Embodiment 3: The engineered CCL3 promoter according to embodiment 1 or embodiment 2, wherein the at least one nucleotide motif comprises a motif having a sequence within the nucleotide sequence of SEQ ID NO: 132, wherein the motif comprises a sequence selected from the group consisting of: position 566 to position 576 of SEQ ID NO: 132, position 674 to position 695 of SEQ ID NO: 132, position 820 to position 832 of SEQ ID NO: 132, position 1089 to position 1105 of SEQ ID NO: 132, position 1127 to position 1141 of SEQ ID NO: 132, position 1184 to position 1199 of SEQ ID NO: 132, position 1475 to position 1500 of SEQ ID NO: 132, position 1534 to position 1544 of SEQ ID NO: 132, position 1553 to position 1595 of SEQ ID NO: 132, position 1634 to position 1674 of SEQ ID NO: 132, position 1681 to position 1692 of SEQ ID NO: 132, and position 1982 to position 1998 of SEQ ID NO: 132.
Embodiment 4: The engineered CCL3 promoter according to any one of embodiments 1-3, wherein the ablation comprises a substitution or deletion of one or more nucleotides of the at least one nucleotide motif.
Embodiment 5: The engineered CCL3 promoter according to any one of embodiments 1-4, wherein the at least one nucleotide motif comprises a motif having a sequence within the nucleotide sequence of SEQ ID NO: 132, wherein the motif corresponds to position 566 to position 576 of SEQ ID NO: 132.
Embodiment 6: The engineered CCL3 promoter according to embodiment 5, wherein the ablation comprises a nucleotide substitution comprising the sequence GTACCAGAATA (SEQ ID NO:191) from position 566 to position 576 of SEQ ID NO:132.
Embodiment 7: The engineered CCL3 promoter according to embodiment 5, wherein the ablation comprises nucleotide deletions of position 566 to position 576 of SEQ ID NO:132.
Embodiment 8: The engineered CCL3 promoter according to any one of embodiments 1-7, wherein the at least one nucleotide motif comprises a motif having a sequence within the nucleotide sequence of SEQ ID NO: 132, wherein the motif corresponds to position 674 to position 695 of SEQ ID NO:132.
Embodiment 9: The engineered CCL3 promoter according to embodiment 8, wherein the ablation comprises a nucleotide substitution comprising the sequence TATATACCAGCGAGTTCGATAA (SEQ ID NO:193) from position 674 to position 695 of SEQ ID NO:132.
Embodiment 10: The engineered CCL3 promoter according to embodiment 8, wherein the ablation comprises nucleotide deletions of position 674 to position 695 of SEQ ID NO:132.
Embodiment 11: The engineered CCL3 promoter according to any one of embodiments 1-10, wherein the at least one nucleotide motif comprises a motif having a sequence within the nucleotide sequence of SEQ ID NO: 132, wherein the motif corresponds to position 820 to position 832 of SEQ ID NO:132.
Embodiment 12: The engineered CCL3 promoter according to embodiment 11, wherein the ablation comprises a nucleotide substitution comprising the sequence ACGAAGCAATACT (SEQ ID NO:195) from position 820 to position 832 of SEQ ID NO:132.
Embodiment 13: The engineered CCL3 promoter according to embodiment 11, wherein the ablation comprises nucleotide deletions of position 820 to position 832 of SEQ ID NO:132.
Embodiment 14: The engineered CCL3 promoter according to any one of embodiments 1-13, wherein the at least one nucleotide motif comprises a motif having a sequence within the nucleotide sequence of SEQ ID NO:132, wherein the motif corresponds to position 1089 to position 1105 of SEQ ID NO:132.
Embodiment 15: The engineered CCL3 promoter according to embodiment 14, wherein the ablation comprises a nucleotide substitution comprising the sequence TCTGTATAAAGCTCGTA (SEQ ID NO:199) from position 1089 to position 1105 of SEQ ID NO:132.
Embodiment 16: The engineered CCL3 promoter according to embodiment 14, wherein the ablation comprises nucleotide deletions of position 1089 to position 1105 of SEQ ID NO:132.
Embodiment 17: The engineered CCL3 promoter according to any one of embodiments 1-16, wherein the at least one nucleotide motif comprises a motif having a sequence within the nucleotide sequence of SEQ ID NO: 132, wherein the motif corresponds to position 1127 to position 1141 of SEQ ID NO:132.
Embodiment 18: The engineered CCL3 promoter according to embodiment 17, wherein the ablation comprises a nucleotide substitution comprising the sequence CCATAGTGAGGAAAT (SEQ ID NO:201) from position 1127 to position 1141 of SEQ ID NO:132.
Embodiment 19: The engineered CCL3 promoter according to embodiment 17, wherein the ablation comprises nucleotide deletions of position 1127 to position 1141 of SEQ ID NO:132.
Embodiment 20: The engineered CCL3 promoter according to any one of embodiments 1-19, wherein the at least one nucleotide motif comprises a motif having a sequence within the nucleotide sequence of SEQ ID NO:132, wherein the motif corresponds to position 1184 to position 1199 of SEQ ID NO:132.
Embodiment 21: The engineered CCL3 promoter according to embodiment 20, wherein the ablation comprises a nucleotide substitution comprising the sequence GTTAAGCATACTAAAC (SEQ ID NO:203) from position 1184 to position 1199 of SEQ ID NO:132.
Embodiment 22: The engineered CCL3 promoter according to embodiment 20, wherein the ablation comprises nucleotide deletions of position 1184 to position 1199 of SEQ ID NO:132.
Embodiment 23: The engineered CCL3 promoter according to any one of embodiments 1-22, wherein the at least one nucleotide motif comprises a motif having a sequence within the nucleotide sequence of SEQ ID NO: 132, wherein the motif corresponds to position 1475 to position 1500 of SEQ ID NO:132.
Embodiment 24: The engineered CCL3 promoter according to embodiment 23, wherein the ablation comprises a nucleotide substitution comprising the sequence CCGATCTCTAGTTAAGTTAGCTGTAT (SEQ ID NO:217) from position 1475 to position 1500 of SEQ ID NO:132.
Embodiment 25: The engineered CCL3 promoter according to embodiment 23, wherein the ablation comprises nucleotide deletions of position 1475 to position 1500 of SEQ ID NO:132.
Embodiment 26: The engineered CCL3 promoter according to any one of embodiments 1-25, wherein the at least one nucleotide motif comprises a motif having a sequence within the nucleotide sequence of SEQ ID NO:132, wherein the motif corresponds to position 1534 to position 1544 of SEQ ID NO:132.
Embodiment 27: The engineered CCL3 promoter according to embodiment 26, wherein the ablation comprises a nucleotide substitution comprising the sequence GTAGAACTCTT (SEQ ID NO:219) from position 1534 to position 1544 of SEQ ID NO:132.
Embodiment 28: The engineered CCL3 promoter according to embodiment 26, wherein the ablation comprises nucleotide deletions of position 1534 to position 1544 of SEQ ID NO:132.
Embodiment 29: The engineered CCL3 promoter according to any one of embodiments 1-28, wherein the at least one nucleotide motif comprises a motif having a sequence within the nucleotide sequence of SEQ ID NO:132, wherein the motif corresponds to position 1553 to position 1595 of SEQ ID NO:132.
Embodiment 30: The engineered CCL3 promoter according to embodiment 29, wherein the ablation comprises a nucleotide substitution comprising the sequence AATACCTTGGTGGAGCTCATCTAATATCTTCATATTACCTCCA (SEQ ID NO:221) from position 1553 to position 1595 of SEQ ID NO:132.
Embodiment 31: The engineered CCL3 promoter according to embodiment 29, wherein the ablation comprises nucleotide deletions of position 1553 to position 1595 of SEQ ID NO:132.
Embodiment 32: The engineered CCL3 promoter according to any one of embodiments 1-31, wherein the at least one nucleotide motif comprises a motif having a sequence within the nucleotide sequence of SEQ ID NO: 132, wherein the motif corresponds to position 1634 to position 1645 of SEQ ID NO:132.
Embodiment 33: The engineered CCL3 promoter according to embodiment 32, wherein the ablation comprises a nucleotide substitution comprising the sequence CGATTGAACAAA (SEQ ID NO:223) from position 1634 to position 1645 of SEQ ID NO:132.
Embodiment 34: The engineered CCL3 promoter according to embodiment 32, wherein the ablation comprises nucleotide deletions of position 1634 to position 1645 of SEQ ID NO:132.
Embodiment 35: The engineered CCL3 promoter according to any one of embodiments 1-34, wherein the at least one nucleotide motif comprises a motif having a sequence within the nucleotide sequence of SEQ ID NO: 132, wherein the motif corresponds to position 1663 to position 1674 of SEQ ID NO:132.
Embodiment 36: The engineered CCL3 promoter according to embodiment 35, wherein the ablation comprises a nucleotide substitution comprising the sequence TATACCTTGGTT (SEQ ID NO:225) from position 1663 to position 1674 of SEQ ID NO:132.
Embodiment 37: The engineered CCL3 promoter according to embodiment 35, wherein the ablation comprises nucleotide deletions of position 1663 to position 1674 of SEQ ID NO:132.
Embodiment 38: The engineered CCL3 promoter according to any one of embodiments 1-37, wherein the at least one nucleotide motif comprises a motif having a sequence within the nucleotide sequence of SEQ ID NO:132, wherein the motif corresponds to position 1681 to position 1692 of SEQ ID NO:132.
Embodiment 39: The engineered CCL3 promoter according to embodiment 38, wherein the ablation comprises a nucleotide substitution comprising the sequence ATTGCGTCAATT (SEQ ID NO:227) from position 1681 to position 1692 of SEQ ID NO:132.
Embodiment 40: The engineered CCL3 promoter according to embodiment 39, wherein the engineered CCL3 promoter comprises the polynucleotide of SEQ ID NO: 4.
Embodiment 41: The engineered CCL3 promoter according to embodiment 38, wherein the ablation comprises nucleotide deletions of position 1681 to position 1692 of SEQ ID NO:132.
Embodiment 42: The engineered CCL3 promoter according to any one of embodiments 1-40, wherein the at least one nucleotide motif comprises a motif having a sequence within the nucleotide sequence of SEQ ID NO: 132, wherein the motif corresponds to position 1982 to position 1998 of SEQ ID NO:132.
Embodiment 43: The engineered CCL3 promoter according to embodiment 42, wherein the ablation comprises a nucleotide substitution comprising the sequence GGAAGTAGCTTGTTTAA (SEQ ID NO:241) from position 1982 to position 1998 of SEQ ID NO:132.
Embodiment 44: The engineered CCL3 promoter according to embodiment 42, wherein the ablation comprises nucleotide deletions of position 1982 to position 1998 of SEQ ID NO:132.
Embodiment 45: The engineered CCL3 promoter according to any one of embodiments 1-44, wherein the ablation comprises an ablation of at least two nucleotide motifs.
Embodiment 46: The engineered CCL3 promoter according to any one of embodiments 1-45, wherein the ablation comprises an ablation of at least three nucleotide motifs.
Embodiment 47: The engineered CCL3 promoter according to any one of embodiments 1-46, wherein the ablation comprises an ablation of at least four nucleotide motifs.
Embodiment 48: The engineered CCL3 promoter according to any one of embodiments 1-47, wherein the ablation comprises an ablation of at least five nucleotide motifs.
Embodiment 49: The engineered CCL3 promoter according to any one of embodiments 1-48, wherein the ablation comprises an ablation of at least six nucleotide motifs.
Embodiment 50: The engineered CCL3 promoter according to embodiment 49, wherein the at least six nucleotide motifs comprise:
a. a nucleotide motif corresponding to positions 566-576 of SEQ ID NO:132,
b. a nucleotide motif corresponding to positions 674-695 of SEQ ID NO:132,
c. a nucleotide motif corresponding to positions 820-832 of SEQ ID NO:132,
d. a nucleotide motif corresponding to positions 1089-1105 of SEQ ID NO:132,
e. a nucleotide motif corresponding to positions 1127-1141 of SEQ ID NO:132, and
f. a nucleotide motif corresponding to positions 1184-1199 of SEQ ID NO:132.
Embodiment 51: The engineered CCL3 promoter according to embodiment 50, wherein the ablation of the at least six nucleotide motifs comprises a deletion corresponding to position 566 to position 1199 of SEQ ID NO:132.
Embodiment 52: The engineered CCL3 promoter according to embodiment 50, wherein the ablation of the at least six nucleotide motifs comprise a deletion corresponding to position 550 to position 1259 of SEQ ID NO:132.
Embodiment 53: The engineered CCL3 promoter according to embodiment 50 or embodiment 51, wherein the ablation further comprises an ablation of a nucleotide motif corresponding to positions 1553 to position 1595 of SEQ ID NO:132.
Embodiment 54: The engineered CCL3 promoter according to embodiment 52, wherein the ablation of the nucleotide motif corresponding to positions 1553 to position 1595 of SEQ ID NO:132 comprises a deletion of the nucleotide motif.
Embodiment 55: The engineered CCL3 promoter according to embodiment 52 or embodiment 54, wherein the ablation further comprises an ablation of a nucleotide motif corresponding to positions 1681 to position 1692 of SEQ ID NO:132.
Embodiment 56: The engineered CCL3 promoter according to embodiment 55, wherein the ablation comprises a nucleotide substitution comprising the sequence ATTGCGTCAATT (SEQ ID NO:227) from position 1681 to position 1692 of SEQ ID NO:132.
Embodiment 57: The engineered CCL3 promoter according to embodiment 56, wherein the engineered CCL3 promoter comprises the polynucleotide sequence of SEQ ID NO: 2.
Embodiment 58: The engineered CCL3 promoter according to embodiment 56, wherein the ablation of the nucleotide motif corresponding to positions 1681-1692 of SEQ ID NO:132 comprises a deletion of the nucleotide motif.
Embodiment 59: The engineered CCL3 promoter according to any one of embodiments 52-57, wherein the ablation further comprises an ablation of a nucleotide motif corresponding to positions 1475 to position 1500 of SEQ ID NO:132.
Embodiment 60: The engineered CCL3 promoter according to embodiment 59, wherein the ablation of the nucleotide motif corresponding to positions 1475 to position 1500 of SEQ ID NO:132 comprises a deletion of the nucleotide motif.
Embodiment 61: The engineered CCL3 promoter according to embodiment 57, wherein the engineered CCL3 promoter comprises the polynucleotide sequence of SEQ ID NO: 1.
Embodiment 62: The engineered CCL3 promoter according to any one of embodiments 1-60, wherein the ablation comprises an ablation of at least nine nucleotide motifs.
Embodiment 63: The engineered CCL3 promoter according to embodiment 61, wherein the at least nine nucleotide motifs comprise:
a nucleotide motif corresponding to positions 566-576 of SEQ ID NO:132,
a nucleotide motif corresponding to positions 674-695 of SEQ ID NO:132,
a nucleotide motif corresponding to positions 820-832 of SEQ ID NO:132,
a nucleotide motif corresponding to positions 1089-1105 of SEQ ID NO:132,
a nucleotide motif corresponding to positions 1127-1141 of SEQ ID NO:132,
a nucleotide motif corresponding to positions 1184-1199 of SEQ ID NO:132,
a nucleotide motif corresponding to positions 1475-1500 of SEQ ID NO:132,
a nucleotide motif corresponding to positions 1553-1595 of SEQ ID NO:132, and
a nucleotide motif corresponding to positions 1681-1692 of SEQ ID NO:132.
Embodiment 64: The engineered CCL3 promoter according to embodiment 63, wherein the ablation comprises a deletion of each of the at least nine nucleotide motifs.
Embodiment 65: The engineered CCL3 promoter according to embodiment 62 or embodiment 63, wherein the engineered CCL3 promoter comprises the polynucleotide sequence of SEQ ID NO: 3.
Embodiment 66: The engineered CCL3 promoter according to embodiment 62, wherein the at least nine nucleotide motifs comprise:
a nucleotide motif corresponding to positions 566-576 of SEQ ID NO:132,
a nucleotide motif corresponding to positions 674-695 of SEQ ID NO:132,
a nucleotide motif corresponding to positions 820-832 of SEQ ID NO:132,
a nucleotide motif corresponding to positions 911-926 of SEQ ID NO:132,
a nucleotide motif corresponding to positions 1089-1105 of SEQ ID NO:132,
a nucleotide motif corresponding to positions 1127-1141 of SEQ ID NO:132,
a nucleotide motif corresponding to positions 1184-1199 of SEQ ID NO:132,
a nucleotide motif corresponding to positions 1211-1223 of SEQ ID NO:132, and
a nucleotide motif corresponding to positions 1236-1248 of SEQ ID NO:132.
Embodiment 67: The engineered CCL3 promoter according to embodiment 62, wherein the ablation of the at least nine nucleotide motifs comprises a deletion corresponding to positions 556 to position 1248 of SEQ ID NO:132.
Embodiment 68: The engineered CCL3 promoter according to embodiment 62, wherein the ablation of the at least nine nucleotide motifs comprises a deletion corresponding to positions 550 to position 1250 of SEQ ID NO:132.
Embodiment 69: The engineered CCL3, promoter according to embodiment 68, wherein the ablation further comprises an ablation of a nucleotide motif corresponding to positions 1425 to position 1445 of SEQ ID NO: 132.
Embodiment 70: The engineered CCL3 promoter according to embodiment 69, wherein the ablation of the nucleotide motif corresponding to positions 1425-1445 of SEQ ID NO:132 comprises a deletion of the nucleotide motif.
Embodiment 71: The engineered CCL3, promoter according to any one of embodiments 68-70, wherein the ablation further comprises an ablation of a nucleotide motif corresponding to positions 1475 to position 1500 of SEQ ID NO: 132.
Embodiment 72: The engineered CCL3 promoter according to embodiment 71, wherein the ablation of the nucleotide motif corresponding to positions 1475-1500 of SEQ ID NO:132 comprises a deletion of the nucleotide motif.
Embodiment 73: The engineered CCL3 promoter according to any one of embodiments 68-72, wherein the ablation further comprises an ablation of a nucleotide motif corresponding to positions 1553 to position 1595 of SEQ ID NO: 132.
Embodiment 74: The engineered CCL3 promoter according to embodiment 73, wherein the ablation of the nucleotide motif corresponding to positions 1553-1595 of SEQ ID NO:132 comprises a deletion of the nucleotide motif.
Embodiment 75: The engineered CCL3 promoter according to any one of embodiments 68-74, wherein the ablation further comprises an ablation of a nucleotide motif corresponding to positions 1663 to position 1679 of SEQ ID NO: 132.
Embodiment 76: The engineered CCL3 promoter according to embodiment 75, wherein the ablation of the nucleotide motif corresponding to positions 1663 to position 1679 of SEQ ID NO:132 comprises a deletion of the nucleotide motif.
Embodiment 77: The engineered CCL3 promoter according to any one of embodiments 68-76 wherein the at least one nucleotide motif comprises a motif having a sequence within the nucleotide sequence of SEQ ID NO:132, wherein the motif corresponds to position 1681 to position 1692 of SEQ ID NO:132.
Embodiment 78: The engineered CCL3 promoter according to embodiment 77, wherein the ablation comprises a nucleotide substitution comprising the sequence ATTGCGTCAATT (SEQ ID NO:227) from position 1681 to position 1692 of SEQ ID NO:132.
Embodiment 79: The engineered CCL3 promoter according to embodiment 78, wherein the CCL3 promoter comprises SEQ ID NO: 242.
Embodiment 80: The engineered CCL3 promoter according to any one of embodiments 68-72, wherein the ablation comprises a deletion of at least two nucleotide motifs according to to position 1425 to position 1500 of SEQ ID NO: 132.
Embodiment 81: The engineered CCL3 promoter according to embodiment 80, wherein the ablation further comprises an ablation of a nucleotide motif corresponding to positions 1553 to position 1595 of SEQ ID NO: 132.
Embodiment 82: The engineered CCL3 promoter according to embodiment 81, wherein the ablation of the nucleotide motif corresponding to positions 1553-1595 of SEQ ID NO:132 comprises a deletion of the nucleotide motif.
Embodiment 83: The engineered CCL3 promoter according to any one of embodiments 80-82, wherein the ablation further comprises an ablation of a nucleotide motif corresponding to positions 1663 to position 1679 of SEQ ID NO: 132.
Embodiment 84: The engineered CCL3 promoter according to embodiment 83, wherein the ablation of the nucleotide motif corresponding to positions 1663-1679 of SEQ ID NO:132 comprises a deletion of the nucleotide motif.
Embodiment 85: The engineered CCL3 promoter according to any one of embodiments 80-84, wherein the at least one nucleotide motif comprises a motif having a sequence within the nucleotide sequence of SEQ ID NO:132, wherein the motif corresponds to position 1681 to position 1692 of SEQ ID NO:132.
Embodiment 86: The engineered CCL3 promoter according to embodiment 85, wherein the ablation comprises a nucleotide substitution comprising the sequence ATTGCGTCAATT (SEQ ID NO:227) from position 1681 to position 1692 of SEQ ID NO:132.
Embodiment 87: The engineered CCL3 promoter according to any one of embodiments 80-86, wherein the ablation further comprises a deletion corresponding to positions 1750 to position 1818 of SEQ ID NO:132.
Embodiment 88: The engineered CCL3 promoter according to embodiment 87, wherein the CCL3 promoter comprises SEQ ID NO: 243.
Embodiment 89: The engineered CCL3 promoter according to embodiment 87 or 88, wherein the ablation further comprises a deletion corresponding to positions 1867 to position 2000 of SEQ ID NO:132.
Embodiment 90: The engineered CCL3 promoter according to embodiment 87, wherein the CCL3 promoter comprises SEQ ID NO: 244.
Embodiment 91: The engineered CCL3 promoter according to any one of embodiments 80-89, wherein the ablation further comprises a deletion to positions 1663 to position 1692 of SEQ ID NO:132
Embodiment 92: The engineered CCL3 promoter according to embodiment 91, wherein the ablation further comprises a deletion corresponding to positions 1750 to position 1818 of SEQ ID NO:132.
Embodiment 93: The engineered CCL3 promoter according to any one of embodiments 91-92, wherein the ablation further comprises a deletion corresponding to positions 1867 to position 2000 of SEQ ID NO:132.
Embodiment 94: The engineered CCL3 promoter according to any one of embodiments 91-93, wherein the CCL3 promoter comprises the polynucleotide sequence of SEQ ID NO: 245.
Embodiment 95: An engineered CCL3 promoter comprising at least one nucleotide motif.
Embodiment 96: The engineered CCL3 promoter according to embodiment 95, wherein the at least one nucleotide motif is selected from the group consisting of
a nucleotide motif corresponding to positions 60-77 of SEQ ID NO:132,
a nucleotide motif corresponding to positions 92-111 of SEQ ID NO:132,
a nucleotide motif corresponding to positions 201-224 of SEQ ID NO:132,
a nucleotide motif corresponding to positions 231-243 of SEQ ID NO:132,
a nucleotide motif corresponding to positions 265-284 of SEQ ID NO:132,
a nucleotide motif corresponding to positions 307-324 of SEQ ID NO:132,
a nucleotide motif corresponding to positions 376-388 of SEQ ID NO:132,
a nucleotide motif corresponding to positions 452-475 of SEQ ID NO:132,
a nucleotide motif corresponding to positions 494-507 of SEQ ID NO:132,
a nucleotide motif corresponding to positions 533-549 of SEQ ID NO:132,
a nucleotide motif corresponding to positions 1260-1288 of SEQ ID NO:132,
a nucleotide motif corresponding to positions 1345-1363 of SEQ ID NO:132,
a nucleotide motif corresponding to positions 1534-1544 of SEQ ID NO:132,
a nucleotide motif corresponding to positions 1634-1645 of SEQ ID NO:132, and
a nucleotide motif corresponding to positions 1840-1861 of SEQ ID NO:132.
Embodiment 97: The engineered CCL3 promoter according to embodiment 95, comprising at least fifteen nucleotide motifs comprising:
a nucleotide motif corresponding to positions 60-77 of SEQ ID NO:132,
a nucleotide motif corresponding to positions 92-111 of SEQ ID NO:132,
a nucleotide motif corresponding to positions 201-224 of SEQ ID NO:132,
a nucleotide motif corresponding to positions 231-243 of SEQ ID NO:132,
a nucleotide motif corresponding to positions 265-284 of SEQ ID NO:132,
a nucleotide motif corresponding to positions 307-324 of SEQ ID NO:132,
a nucleotide motif corresponding to positions 376-388 of SEQ ID NO:132,
a nucleotide motif corresponding to positions 452-475 of SEQ ID NO:132,
a nucleotide motif corresponding to positions 494-507 of SEQ ID NO:132,
a nucleotide motif corresponding to positions 533-549 of SEQ ID NO:132,
a nucleotide motif corresponding to positions 1260-1288 of SEQ ID NO:132,
a nucleotide motif corresponding to positions 1345-1363 of SEQ ID NO:132,
a nucleotide motif corresponding to positions 1534-1544 of SEQ ID NO:132,
a nucleotide motif corresponding to positions 1634-1645 of SEQ ID NO:132, and
a nucleotide motif corresponding to positions 1840-1861 of SEQ ID NO:132.
Embodiment 98: The engineered CCL3 promoter according to any one of embodiments 95-97, wherein the CCL3 promoter comprises the polynucleotide sequence of SEQ ID NO: 246.
Embodiment 99: An engineered CCL3 promoter comprising the polynucleotide sequence of SEQ ID NO: 1.
Embodiment 100: An engineered CCL3 promoter comprising the polynucleotide sequence of SEQ ID NO: 2.
Embodiment 101: An engineered CCL3 promoter comprising the polynucleotide sequence of SEQ ID NO: 3.
Embodiment 102: An engineered CCL3 promoter comprising the polynucleotide sequence of SEQ ID NO: 4.
Embodiment 103: An engineered CCL3 promoter comprising the polynucleotide sequence of SEQ ID NO: 242.
Embodiment 104: An engineered CCL3 promoter comprising the polynucleotide sequence of SEQ ID NO: 243.
Embodiment 105: An engineered CCL3 promoter comprising the polynucleotide sequence of SEQ ID NO: 244.
Embodiment 106: An engineered CCL3 promoter comprising the polynucleotide sequence of SEQ ID NO: 245.
Embodiment 107: An engineered CCL3 promoter comprising the polynucleotide sequence of SEQ ID NO: 246.
Embodiment 108: A heterologous construct comprising the engineered CCL3 promoter according to any one of embodiments 1-107 operably linked to a polynucleotide comprising a polynucleotide sequence encoding a polypeptide.
Embodiment 109: The heterologous construct according to embodiment 108, wherein the polypeptide comprises at least one effector molecule.
Embodiment 110: The heterologous construct according to embodiment 108 or embodiment 109, wherein the polypeptide comprises a first effector molecule and a second effector molecule.
Embodiment 111: The heterologous construct according to embodiment 110, wherein the polynucleotide comprises a polynucleotide sequence encoding the first effector molecule, a linker polynucleotide sequence, and a polynucleotide sequence encoding the second effector.
Embodiment 112: The heterologous construct according to embodiment 111, wherein the linker polynucleotide sequence encodes one or more 2A ribosome skipping elements.
Embodiment 113: The heterologous construct according to embodiment 112, wherein the one or more 2A ribosome skipping elements comprise elements that are each selected from the group consisting of: P2A, T2A, E2A, and F2A.
Embodiment 114: The heterologous construct according to any one of embodiments 109-113, wherein the at least one effector molecule is selected from the group consisting of: a therapeutic class, wherein the therapeutic class is selected from the group consisting of: a cytokine, a chemokine, a homing molecule, a growth factor, a co-activation molecule, a tumor microenvironment modifier, a receptor, a ligand, an antibody, a peptide, and an enzyme.
Embodiment 115: The heterologous construct according to any one of embodiments 109-114, wherein each of the at least one effector molecule comprises a cytokine.
Embodiment 116: The heterologous construct according to embodiment 115, wherein the cytokine is selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.
Embodiment 117: The heterologous construct according to any one of embodiments 109-114, wherein each of the at least one effector molecule comprises a chemokine.
Embodiment 118: The heterologous construct according to embodiment 117, wherein the chemokine is selected from the group consisting of: CCL21a, CXCL10, CXCL11, CXCL13, a CXCL10-CXCL11 fusion protein, CCL19, CXCL9, and XCL1.
Embodiment 119: The heterologous construct according to any one of embodiments 109-114, wherein each of the at least one effector molecule comprises a homing molecule.
Embodiment 120: The heterologous construct according to embodiment 119, wherein the homing molecule is selected from the group consisting of: anti-integrin alpha4, beta7; anti-MAdCAM; CCR9; CXCR4; SDFI; MMP-2; CXCR1; CXCR7; CCR2; CCR4; and GPR15.
Embodiment 121: The heterologous construct according to any one of embodiments 109-114, wherein each of the at least one effector molecule comprises a growth factor.
Embodiment 122: The heterologous construct according to embodiment 121, wherein the growth factor is selected from the group consisting of: FLT3L and GM-CSF.
Embodiment 123: The heterologous construct according to any one of embodiments 109-114, wherein each of the at least one effector molecule comprises a co-activation molecule.
Embodiment 124: The heterologous construct according to embodiment 123, wherein the co-activation molecule is selected from the group consisting of: c-Jun, 4-1BBL and CD40L.
Embodiment 125: The heterologous construct according to any one of embodiments 109-114, wherein each of the at least one effector molecule comprises a tumor microenvironment modifier.
Embodiment 126: The heterologous construct according to embodiment 123, wherein the tumor microenvironment modifier is selected from the group consisting of: an adenosine deaminase, a TGFbeta inhibitor, an immune checkpoint inhibitor, a VEGF inhibitor, and an HPGE2.
Embodiment 127: The heterologous construct according to any one of embodiments 110-114, wherein each of the first effector molecule and the second effector molecule is from a separate therapeutic class.
Embodiment 128: The heterologous construct according to any one of embodiments 109-127, wherein each of the at least one effector molecule is a human-derived effector molecule.
Embodiment 129: A vector comprising the heterologous construct according to any one of embodiments 108-128.
Embodiment 130: A dual expression vector comprising the heterologous construct according to any one of embodiments 108-128 and a second construct comprising a polynucleotide sequence encoding an activating immune receptor.
Embodiment 131: The dual expression vector according to embodiment 130, wherein the activating immune receptor comprises an antigen recognizing receptor.
Embodiment 132: The dual expression vector according to embodiment 131, wherein the antigen recognizing receptor comprises a T Cell Receptor (TCR).
Embodiment 133: The dual expression vector according to embodiment 132, wherein the TCR is an endogenous T cell receptor or an exogenous T cell receptor.
Embodiment 134: The dual expression vector according to embodiment 132 or embodiment 133, wherein the TCR is an endogenous T cell receptor.
Embodiment 135: The dual expression vector according to embodiment 132 or embodiment 133, wherein the TCR is an exogenous T cell receptor.
Embodiment 136: The dual expression vector according to embodiment 131, wherein the antigen recognizing receptor comprises a Chimeric Antigen Receptor (CAR).
Embodiment 137: An immunoresponsive cell comprising the heterologous construct according to any one of embodiments 108-128, the vector according to embodiment 129, or the dual expression vector according to any one of embodiments 130-136.
Embodiment 138: The immunoresponsive cell according to embodiment 137, wherein the immunoresponsive cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.
Embodiment 139: The immunoresponsive cell according to embodiment 137, wherein the immunoresponsive cells is a T cell.
Embodiment 140: The immunoresponsive cell according to embodiment 137, wherein the immunoresponsive cell is an NK cell.
Embodiment 141: The immunoresponsive cell according to any one of embodiments 137-140, wherein the immunoresponsive cell expresses an activating immune receptor.
Embodiment 142: The immunoresponsive cell according to embodiment 141, wherein the activating immune receptor comprises an antigen recognizing receptor.
Embodiment 143: The immunoresponsive cell according to embodiment 142, wherein the antigen recognizing receptor comprises a T Cell Receptor (TCR).
Embodiment 144: The immunoresponsive cell according to embodiment 143, wherein the TCR is an endogenous T cell receptor or an exogenous T cell receptor.
Embodiment 145: The immunoresponsive cell according to embodiment 143 or 144, wherein the TCR is an endogenous T cell receptor.
Embodiment 146: The immunoresponsive cell according to embodiment 143 or 144, wherein the TCR is an exogenous T cell receptor.
Embodiment 147: The immunoresponsive cell according to embodiment 142, wherein the antigen recognizing receptor comprises a Chimeric Antigen Receptor (CAR).
Embodiment 148: The immunoresponsive cell according to embodiment 142, wherein the antigen recognizing receptor comprises an activating NK cell receptor.
Embodiment 149: The immunoresponsive cell according to embodiment 148, wherein the activating NK cell receptor is selected from the group consisting of NKG2D, NKp30, NKp44, NKp46, and ITAM-containing Killer cell Ig-like receptors (KIRs).
Embodiment 150: The immunoresponsive cell according to embodiment 148 or 149, wherein the activating NK cell receptor is NKG2D.
Embodiment 151: The immunoresponsive cell according to embodiment 148 or 149, wherein the activating NK cell receptor is NKp30.
Embodiment 152: The immunoresponsive cell according to embodiment 148 or 149, wherein the activating NK cell receptor is NKp44.
Embodiment 153: The immunoresponsive cell according to embodiment 148 or 149, wherein the activating NK cell receptor is NKp46.
Embodiment 154: The immunoresponsive cell according to embodiment 148 or 149, wherein the activating NK cell receptor is ITAM-containing KIRs.
Embodiment 155: The immunoresponsive cell according to any one of embodiments 142-147, wherein the immunoresponsive cell comprises a heterologous construct encoding the antigen recognizing receptor.
Embodiment 156: The immunoresponsive cell according to any one of embodiments 137-155, wherein the immunoresponsive cell is autologous.
Embodiment 157: The immunoresponsive cell according to any one of embodiments 137-155, wherein the immunoresponsive cell is allogeneic.
Embodiment 158: A pharmaceutical composition comprising the engineered CCL3 promoter according to any one of embodiments 1-107, the heterologous construct according to any one of embodiments 108 to 128, the vector of embodiment 129, the dual expression vector according to any one of embodiments 130-136, or the immunoresponsive cell according to any one of embodiments 137-157, and a pharmaceutically acceptable carrier, pharmaceutically acceptable excipient, or a combination thereof.
Embodiment 159: A method of increasing expression of a target gene, the method comprising use of the engineered CCL3 promoter of any one of embodiments 1-98, the heterologous construct according to any one of embodiments 108 to 128, the vector of embodiment 129, or the dual expression vector according to any one of embodiments 130-136 to increase expression of the target gene.
Embodiment 160: The method of embodiment 159, wherein the target gene is an immunomodulatory gene.
Embodiment 161: A method of treating a subject in need thereof, the method comprising administering the engineered CCL3 promoter according to any one of embodiments 1-107, the heterologous construct according to any one of embodiments 108 to 128, a therapeutically effective dose of the vector of embodiment 129, the dual expression vector according to any one of embodiments 130-136, the immunoresponsive cell according to any one of embodiments 137-157, or the pharmaceutical composition according to embodiment 158.
Embodiment 162: A method of stimulating a cell-mediated immune response in a subject, the method comprising administering to a subject the engineered CCL3 promoter according to any one of embodiments 1-107, the heterologous construct according to any one of embodiments 108 to 128, a therapeutically effective dose of the vector of embodiment 129, the dual expression vector according to any one of embodiments 130-136, the immunoresponsive cell according to any one of embodiments 137-157 or the pharmaceutical composition according to embodiment 158.
Embodiment 163: A method of reducing tumor volume in a subject, the method comprising administering to a subject having a tumor a composition comprising the engineered CCL3 promoter according to any one of embodiments 1-107, the heterologous construct according to any one of embodiments 108 to 128, the vector of embodiment 129, the dual expression vector according to any one of embodiments 130-136, the immunoresponsive cell according to any one of embodiments 137-157 or the pharmaceutical composition according to embodiment 158.
Embodiment 164: A method of providing an anti-tumor immunity in a subject, the method comprising administering to a subject in need thereof the engineered CCL3 promoter according to any one of embodiments 1-107, the heterologous construct according to any one of embodiments 108 to 128, a therapeutically effective dose of the vector of embodiment 56, the dual expression vector according to any one of embodiments 130-136, the immunoresponsive cell according to any one of embodiments 137-157 or the pharmaceutical composition according to embodiment 158.
Embodiment 165: A method of treating a subject having cancer, the method comprising administering the engineered CCL3 promoter according to any one of embodiments 1-107, the heterologous construct according to any one of embodiments 108 to 128, a therapeutically effective dose of the vector of embodiment 129, the dual expression vector according to any one of embodiments 130-136, the immunoresponsive cell according to any one of embodiments 137-157 or the pharmaceutical composition according to embodiment 158 to the subject in need thereof.
Embodiment 166: The method of any one of embodiments 134-138, wherein the method comprises administering the immunoresponsive cell.
Embodiment 167: A kit for treating and/or preventing a tumor, comprising the immunoresponsive cell according to any one of embodiments 137-157.
Embodiment 168: The kit according to embodiment 167, wherein the kit further comprises written instructions for using the immunoresponsive cell for treating and/or preventing a tumor in a subject.
Embodiment 169: A kit for treating and/or preventing a tumor, comprising the pharmaceutical composition according to embodiment 158.
Embodiment 170: The kit according to embodiment 169, wherein the kit further comprises written instructions for using the pharmaceutical composition for treating and/or preventing a tumor in a subject.

### Examples

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1: Generation of a CCL3 Promoter Ablation Series and Assessment in Immunoresponsive Cells

In order to provide variants of the CCL3 promoter with reduced size, an engineering approach was undertaken using the wild-type CCL3 promoter (SEQ ID NO:132) as template. Each regulatory element (e.g., transcription factor binding motif) was replaced with an artificial nucleotide sequence using standard molecular cloning techniques known in the art. Following introduction of the artificial nucleotide sequence, the new promoter sequence was analyzed bioinformatically to ensure no new binding motif was introduced. A total of 36 CCL3 promoter variants were produced. The sequences of these variants are provided in Table 1.

### Inducibility of CCL3 Promoter Variants in T Cells

The CCL3 promoter variants were then tested for inducibility. Briefly, Pan T cells, isolated from apheresis pack derived peripheral blood mononuclear cells (PBMCs), were incubated with CD3/CD28 antibody tagged-beads at a ratio of 3:1 for activation. After about 24 hours of incubation, the Pan T cells were cotransduced with virus containing the CCL3 promoter driving mKate-PEST virus and an exemplary CAR (comprising a GPC3-binding chimeric antigen receptor (CAR) with CD8a hinge, CD8a transmembrane domain, and 4-1BB/CD3zeta intracellular domains). After about 72 hours, the beads were washed out. About 20,000 transduced Pan T cells were seeded per well onto a 96 well plate and given about 24 hours to adhere. Transduced Pan T cells were activated with the CD3/CD28 beads at a 1:1 ratio for about 48 hours. Activated Pan T cells were then quantified by flow cytometry for mCherry (mKate) and YFP (GPC3) expression.

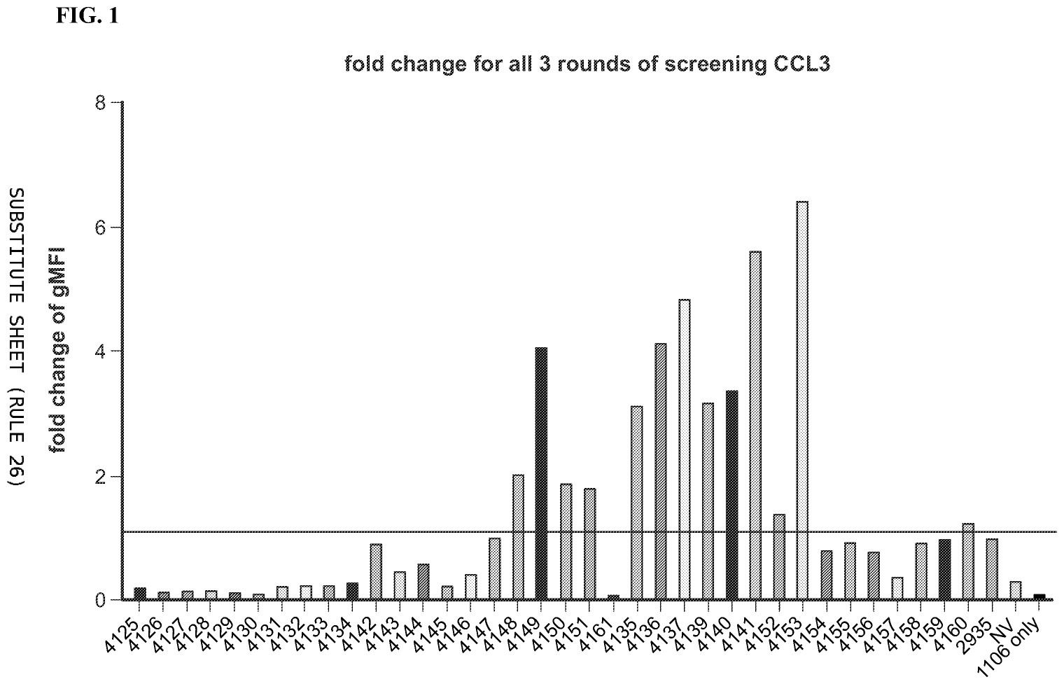

Quantification of fluorescence is shown in FIG. 1, normalized to the wild-type CCL3 promoter (SEQ ID NO:132). The CCL3 promoter variants demonstrating the highest levels of inducibility were 4153 (SEQ ID NO:161), 4141 (SEQ ID NO:149), 4137 (SEQ ID NO:145), 4136 (SEQ ID NO:144), 4149 (SEQ ID NO:157), 4135 (SEQ ID NO:143), 4139 (SEQ ID NO:147), and 4140 (SEQ ID NO:148).

### Inducibility of CCL3 Promoter Variants in Natural Killer (NK) Cells

Natural Killer (NK) cells derived from a human donor were activated with irradiated K562 feeder cells displaying membrane bound IL-21 and membrane bound IL-15. On day 10 after activation, NK cells were transduced with virus. The media was changed after 24 h and then the cells were rested until day 18. The NK cells were transduced with virus for the eight CCL3 promoter variants showing the highest levels of inducibility in T cells (4153, 4141, 4137, 4136, 4149, 4135, 4139, and 4140), as described above. Following about eight days of incubation, the NK cells were activated by co-culturing with Huh7 cells at a 1:1 ratio, and the cells were co-cultured for a total of either about 24 hours or about 48 hours incubation. NK cells, both activated and non-activated, were then assessed for mCherry (mKate) by flow cytometry. Wildtype CCL3 promoter (2935), SFFV mKate (1682), and 2096 (NFAT mKate) were used as controls.

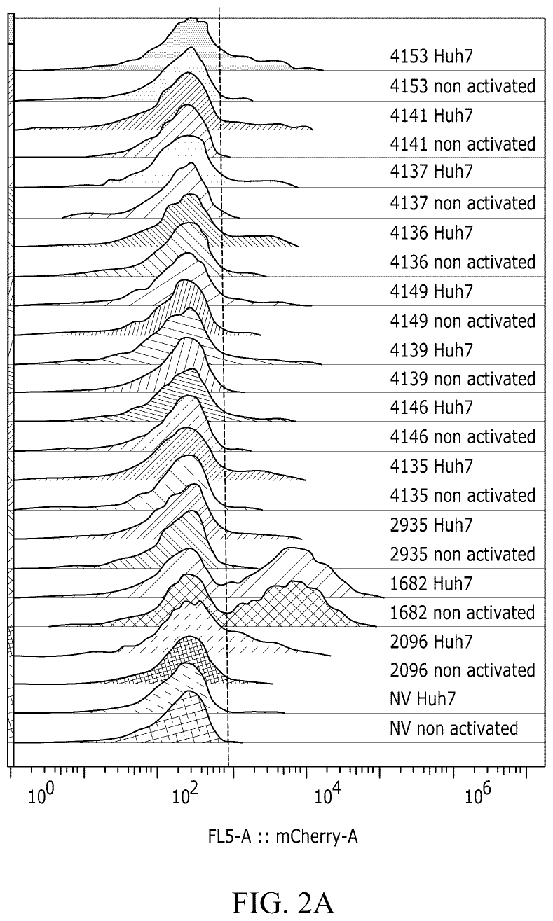

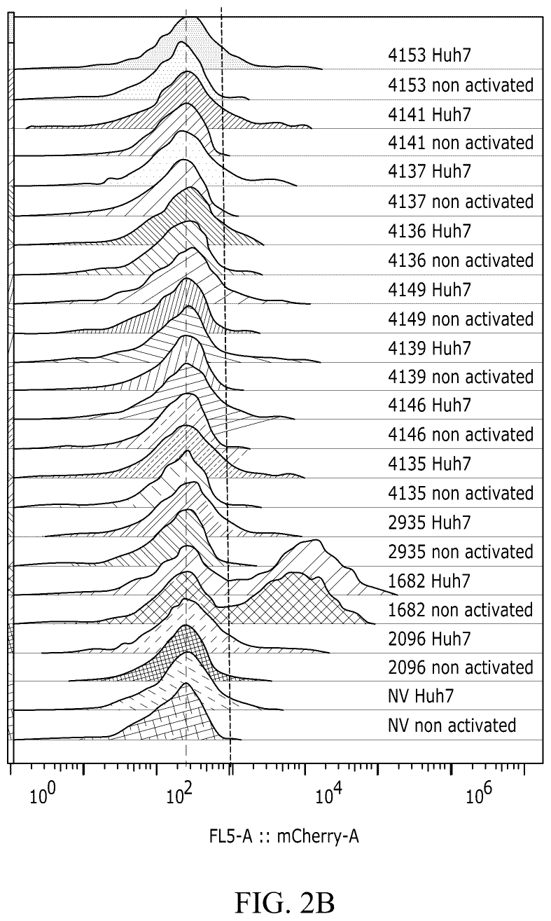

Quantification of the fluorescence signals is shown in FIGS. 2A and 2B. Induction was observed for all constructs following activation for either 24 (FIG. 2A) or 48 hours FIG. 2B.

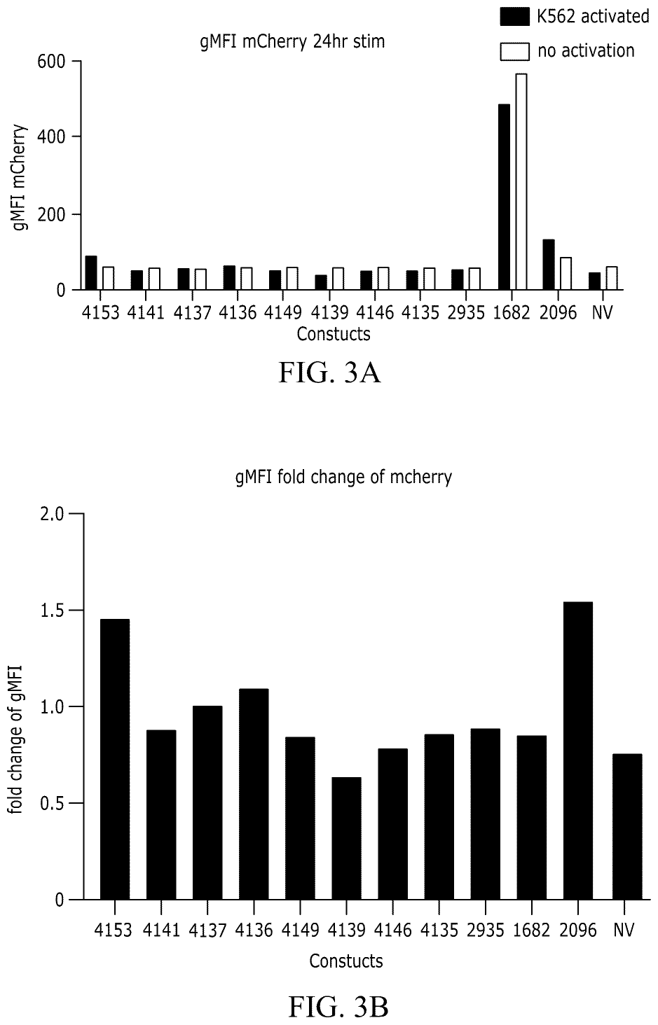

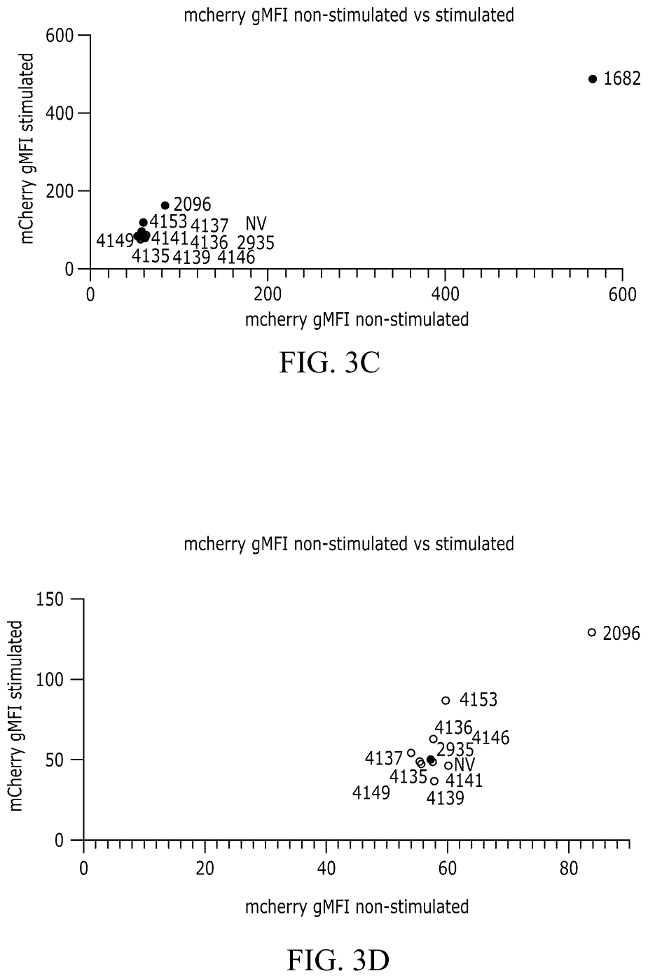

FIG. 3A shows histograms depicting geometric mean fluorescence intensity (gMFI) for mKate (mCherry) of activated and not activated NK cells following 24 hours stimulation. The 4153 promoter variant displayed the highest mCherry-fluorescence fold change as compared to the other CCL3 promoter variants, and the fold change was comparable to that observed in the NFAT promoter (2096) control. Fold change of mCherry is demonstrated in FIG. 3B. As shown in FIGS. 3C and 3D (which has 1682 removed due to its outlier levels of gMFI), which depicts stimulated NK cells plotted against non-stimulated NK cells, variants 4137, 4136, 4153 had the highest gMFI when stimulated for 24 hours and low basal levels of mCherry expression, as compared to control. Also, as can be seen in FIGS. 3C and 3D, the 2096 promoter resulted in higher levels of expression in both stimulated and unstimulated cell, and thus one benefit of the CCL3 promoter variants is inducibility based on NK cell activation but with lower leakiness (i.e., lower baseline expression) than the NFAT promoter.

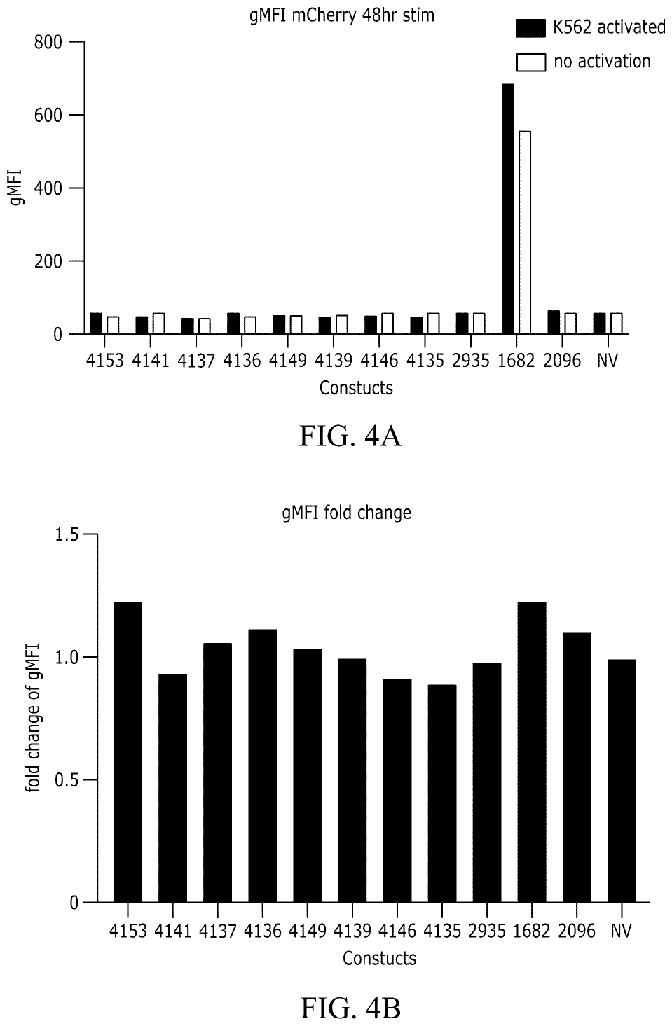

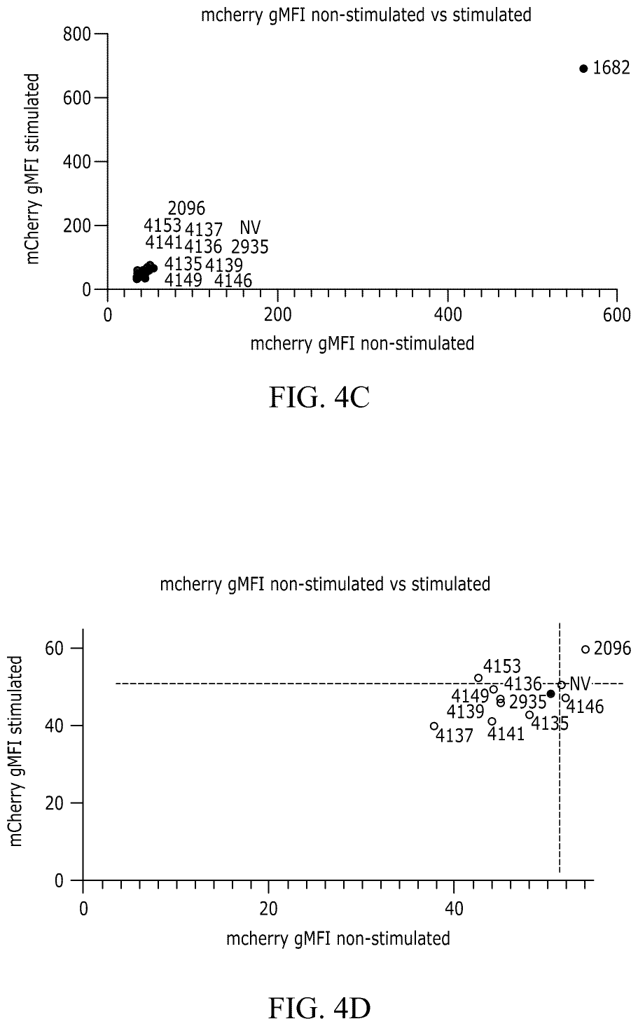

FIG. 4A shows histograms depicting geometric mean fluorescence intensity (gMFI) for mKate (mCherry) of activated and not activated NK cells following 48 hours stimulation. The 4153 promoter variant displayed the highest mCherry-fluorescence fold change as compared to the other CCL3 promoter variants, and the fold change was higher than the CCL3 promoter control (2935). Fold change of mCherry is demonstrated in FIG. 4B. As shown in FIGS. 4C and 4D (which has 1682 removed due to its outlier levels of gMFI), for stimulated plotted against non-stimulated NK cells, variant 4153 displayed the highest gMFI when stimulated for 48 hours and low basal mCherry expression, as compared to control.

While the above studies were initially designed for determining a CCL3 promoter with reduced size, surprisingly, it was observed that ablation of specific nucleotide motifs in the CCL3 promoter provided enhanced inducibility of the variant promoters relative to the wild-type promoter.

### Example 2: Generation of a CCL3 Promoter Deletions and Assessment in Immunoresponsive Cells

In order to reduce the size of CCL3 promoters having enhanced inducibility, deletion variants were engineered, including deletions at positions substituted in ablation patches as provided in Example 1.

A first promoter variant construct, 5958, was engineered to include deletion of the motifs at each ablation patch substituted in the following constructs: 4141, 4137, 4136, 4135, 4139, 4140, 4148, 4150, and 4153. A second promoter variant construct, 5959, was engineered to delete a nucleotide sequence between, but not including, the ablation patches substituted in the 4134 and 4145 constructs (from positions 550 to 1259 of SEQ ID NO: 132), and to replace the wild-type sequence (AGAAACCATTTC SEQ ID NO: 226) with that of the ablation patch substitutions of 4153 (i.e., the nucleotide substitution comprising the sequence ATTGCGTCAATT (SEQ ID NO:227) from position 1681 to position 1692 of SEQ ID NO: 132). A third promoter variant construct, 5960, was engineered to delete a nucleotide sequence between, but not including, the ablation patches substituted in the 4134 and 4145 constructs (from positions 550 to 1259 of SEQ ID NO: 132), to delete the sequences of ablation patch substitutions of 4148 and 4150, and to include the ablation patch substitution sequence of 4153. Each of these deletion-containing constructs included a secreted blue fluorescent protein (sec-BFP) reporter. Additionally, a fourth promoter construct, 5957, was engineered to include the same ablation patch substitution as 4153 and include the sec-BFP reporter. The sequences of these four promoter variants are provided in Table 1.

T cells isolated from apheresis pack derived PBMCs were transduced with the four CCL3 promoter variant constructs described in Example 1 above. T cells were transduced 24 hours after activation, as described above. On day 2 following transduction, BFP expression was assessed by flow cytometry. For each of the four transductions, approximately 70% of cells were BFP positive on day 2 (data not shown). Eleven days following transduction, T cells were again stimulated with Dynabeads coated with anti-CD3 and anti-CD28 agonist antibodies at a bead-to-cell ratio of 1:1 or were unstimulated as a control. Two days prior to bead stimulation, the cells were plated at 200,000 cell/well. Two days following bead stimulation, BFP expression was again assessed, and bead-stimulated versus unstimulated BFP levels were compared for each construct.

Geometric mean fluorescent intensity (gMFI) and fold change between stimulated versus stimulated are shown in FIGS. 5A and 5B, respectively. As shown in FIG. 5B, each of the four constructs had at least a ten-fold increase in BFP expression for the bead-stimulated T cells, as compared to unstimulated cells. This increased inducibility of CCL3 promoters may be achieved by deleting the nucleotide sequences of ablation patches identified in Example 1. Additionally, increased inducibility may be obtained by deleting sequences including and between many of the ablation patches identified in Example 1, which can beneficially reduce the size of the promoter by up to over 700 nucleotides.

### Example 3: Generation of Additional CCL3 Promoter Deletions and Assessment in Immunoresponsive Cells

Additional CCL3 promoter deletions were generated and tested.

A first promoter variant construct, 5957, was generated as previously described in Example 2.

A second promoter variant construct, 7293, was engineered to delete a nucleotide sequence between, but not including, the ablation patches substituted in the 4134 and 4145 constructs (from positions 550 to 1259 of SEQ ID NO: 132), to delete the sequences of ablation patch substitutions of 4147, 4148, 4150, and 4152, and to include the ablation patch substitution sequence of 4153. A third promoter variant construct, 7294, was engineered to delete a nucleotide sequence between, but not including the ablation patches substituted in the 4134 and 4145 constructs (from positions 550 to 1259 of SEQ ID NO: 132), to delete the sequences of ablation patch substitutions of 4150 and 4152, to include the ablation patch substitution sequence of 4153, and to delete a nucleotide sequence between and including the ablation patches in the 4154 and 4156 constructs.

A third promoter variant construct, 7295, was engineered to delete a nucleotide sequence between, but not including the ablation patches substituted in the 4134 and 4145 constructs (from positions 550 to 1259 of SEQ ID NO: 132), to delete the sequences including and between the ablation patch substitutions of 4147 and 4148 (from positions 1425-1500 of SEQ ID NO: 132), to delete the ablation patch substitutions of 4150, to delete the ablation patch substitutions of 4152, to delete the sequences including and between the ablation patch substitutions of 4154 and 4156 (from positions 1750 to 1818 of SEQ ID NO: 132), to delete the sequences including and between the ablation patch substitutions of 4158 and 4160 (from positions 1867 to 2000 of SEQ ID NO: 132), and to include the ablation patch substitution sequence of 4153.

A fourth promoter variant construct, 7296, was engineered to delete a nucleotide sequence between, but not including the ablation patches substituted in the 4134 and 4145 constructs (from positions 550 to 1259 of SEQ ID NO: 132), to delete the sequences including and between the ablation patch substitutions of 4147 and 4148 (from positions 1425-1500 of SEQ ID NO: 132), to delete the ablation patch substitutions of 4150, to delete the sequences including and between the ablation patch substitutions of 4152 and 4156 (from positions 1663 to 1818 of SEQ ID NO: 132), and to delete the sequences including and between the ablation patch substitutions of 4158 and 4160 (from positions 1867 to 2000 of SEQ ID NO: 132). Each of these deletion-containing constructs included a secreted blue fluorescent protein (sec-BFP) reporter. The sequences of these promoter variants are provided in Table 1.

A fifth promoter variant construct, 7297, was engineered to generate a small promoter that contained the ablation patches of 4125, 4126, 4127, 4128, 4129, 4130, 4131, 4132, 4133, 4134, 4145, 4146, 4149, 4151, and 4157.

T cells isolated from apheresis pack derived PBMCs were transduced with the CCL3 promoter variant constructs as previously described. T cells were transduced 24 hours after activation, as described above. On day 2 following transduction, BFP expression was assessed by flow cytometry. For each transduction, approximately 70% of cells were BFP positive on day 2 (data not shown). Eleven days following transduction, T cells were again stimulated with Dynabeads coated with anti-CD3 and anti-CD28 agonist antibodies at a bead-to-cell ratio of 1:1 or were unstimulated as a control. Two days prior to bead stimulation, the cells were plated at 200,000 cell/well. Two days following bead stimulation, BFP expression was again assessed, and bead-stimulated versus unstimulated BFP levels were compared for each construct.

Geometric mean fluorescent intensity (gMFI) and fold change between stimulated versus unstimulated are shown in FIGS. 6A and 6B, respectively. As shown in FIG. 6B, except for construct 7297, the constructs had an increase in BFP expression for the bead-stimulated T cells, as compared to unstimulated cells. This increased inducibility of CCL3 promoters may be achieved by deleting the nucleotide sequences of ablation patches identified in Example 1. Additionally, increased inducibility may be obtained by deleting sequences including and between many of the ablation patches identified in Example 1, which can beneficially reduce the size of the promoter.

### Interpretations

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1.-15. (canceled)

16. An engineered CCL3 promoter comprising
a. at least one nucleotide motif within the nucleotide sequence of SEQ ID NO: 132; and/or
b. an ablation of the at least one nucleotide motif, wherein the ablation increases inducibility of the engineered CCL3 promoter in the presence of an immune cell activation signal, as compared to inducibility of a wild-type CCL3 promoter in the presence of the same immune cell activation signal; wherein the wild-type CCL3 promoter comprises the nucleotide sequence of SEQ ID NO: 132;
wherein the ablation comprises a substitution or deletion of one or more nucleotides of the at least one nucleotide motif, optionally wherein the at least one nucleotide motif comprises a motif having a sequence within the nucleotide sequence of SEQ ID NO: 132.

17. The engineered CCL3 promoter of claim 16, wherein the nucleotide motif comprises a sequence selected from the group consisting of:
a. position 566 to position 576 of SEQ ID NO: 132,
b. position 674 to position 695 of SEQ ID NO: 132,
c. position 820 to position 832 of SEQ ID NO: 132,
d. position 1089 to position 1105 of SEQ ID NO: 132,
e. position 1127 to position 1141 of SEQ ID NO: 132,
f. position 1184 to position 1199 of SEQ ID NO: 132,
g. position 1475 to position 1500 of SEQ ID NO: 132,
h. position 1534 to position 1544 of SEQ ID NO: 132,
i. position 1553 to position 1595 of SEQ ID NO: 132,
j. position 1634 to position 1674 of SEQ ID NO: 132,
k. position 1681 to position 1692 of SEQ ID NO: 132,
l. position 1982 to position 1998 of SEQ ID NO: 132,
m. position 60 to position 77 of SEQ ID NO:132,
n. position 92 to position 111 of SEQ ID NO:132,
o. position 201 to position 224 of SEQ ID NO:132,
p. position 231 to position 243 of SEQ ID NO:132,
q. position 265 to position 284 of SEQ ID NO:132,
r. position 307 to position 324 of SEQ ID NO:132,
s. position 376 to position 388 of SEQ ID NO:132,
t. position 452 to position 475 of SEQ ID NO:132,
u. position 494 to position 507 of SEQ ID NO:132,
v. position 533 to position 549 of SEQ ID NO:132,
w. position 1260 to position 1288 of SEQ ID NO:132,
x. position 1345 to position 1363 of SEQ ID NO:132,
y. position 1534 to position 1544 of SEQ ID NO:132,
z. position 1634 to position 1645 of SEQ ID NO:132, and/or
aa. position 1840 to position 1861 of SEQ ID NO:132.

18. The engineered CCL3 promoter of claim 16, wherein the ablation comprises:
a. a nucleotide substitution comprising the sequence GTACCAGAATA (SEQ ID NO:191) from position 566 to position 576 of SEQ ID NO:132, optionally wherein the ablation comprises nucleotide deletions of position 566 to position 576 of SEQ ID NO:132,
b. a nucleotide substitution comprising the sequence TATATACCAGCGAGTTCGATAA (SEQ ID NO:193) from position 674 to position 695 of SEQ ID NO:132, optionally wherein the ablation comprises nucleotide deletions of position 674 to position 695 of SEQ ID NO:132,
c. a nucleotide substitution comprising the sequence ACGAAGCAATACT (SEQ ID NO:195) from position 820 to position 832 of SEQ ID NO:132, optionally wherein the ablation comprises nucleotide deletions of position 820 to position 832 of SEQ ID NO:132,
d. a nucleotide substitution comprising the sequence TCTGTATAAAGCTCGTA (SEQ ID NO:199) from position 1089 to position 1105 of SEQ ID NO:132, optionally wherein the ablation comprises nucleotide deletions of position 1089 to position 1105 of SEQ ID NO:132,
e. a nucleotide substitution comprising the sequence CCATAGTGAGGAAAT (SEQ ID NO:201) from position 1127 to position 1141 of SEQ ID NO:132, optionally wherein the ablation comprises nucleotide deletions of position 1127 to position 1141 of SEQ ID NO:132,
f. a nucleotide substitution comprising the sequence GTTAAGCATACTAAAC (SEQ ID NO:203) from position 1184 to position 1199 of SEQ ID NO:132, optionally wherein the ablation comprises nucleotide deletions of position 1184 to position 1199 of SEQ ID NO:132,
g. a nucleotide substitution comprising the sequence CCGATCTCTAGTTAAGTTAGCTGTAT (SEQ ID NO:217) from position 1475 to position 1500 of SEQ ID NO:132, optionally wherein the ablation comprises nucleotide deletions of position 1475 to position 1500 of SEQ ID NO:132,
h. a nucleotide substitution comprising the sequence GTAGAACTCTT (SEQ ID NO:219) from position 1534 to position 1544 of SEQ ID NO:132, optionally wherein the ablation comprises nucleotide deletions of position 1534 to position 1544 of SEQ ID NO:132,
i. a nucleotide substitution comprising the sequence AATACCTTGGTGGAGCTCATCTAATATCTTCATATTACCTCCA (SEQ ID NO:221) from position 1553 to position 1595 of SEQ ID NO:132, optionally wherein the ablation comprises nucleotide deletions of position 1553 to position 1595 of SEQ ID NO:132,
j. a nucleotide substitution comprising the sequence CGATTGAACAAA (SEQ ID NO:223) from position 1634 to position 1645 of SEQ ID NO:132, optionally wherein the ablation comprises nucleotide deletions of position 1634 to position 1645 of SEQ ID NO:132,
k. a nucleotide substitution comprising the sequence TATACCTTGGTT (SEQ ID NO:225) from position 1663 to position 1674 of SEQ ID NO:132, optionally wherein the ablation comprises nucleotide deletions of position 1663 to position 1674 of SEQ ID NO:132, and/or
l. a nucleotide substitution comprising the sequence ATTGCGTCAATT (SEQ ID NO:227) from position 1681 to position 1692 of SEQ ID NO:132.

19. An engineered CCL3 promoter comprising a polynucleotide sequence selected from the group consisting of SEQ ID NOs: 1-4 and 242-246.

20. A heterologous construct comprising the engineered CCL3 promoter of claim 16 operably linked to a polynucleotide comprising a polynucleotide sequence encoding a polypeptide, wherein the polypeptide comprises at least one effector molecule.

21. The heterologous construct of claim 20, wherein the at least one effector molecule is selected from a therapeutic class, wherein the therapeutic class is selected from the group consisting of: a cytokine, a chemokine, a homing molecule, a growth factor, a co-activation molecule, a tumor microenvironment modifier, a receptor, a ligand, an antibody, a peptide, and an enzyme.

22. A heterologous construct comprising the engineered CCL3 promoter of claim 19 operably linked to a polynucleotide comprising a polynucleotide sequence encoding a polypeptide, wherein the polypeptide comprises at least one effector molecule.

23. The heterologous construct of claim 22, wherein the at least one effector molecule is selected from a therapeutic class, wherein the therapeutic class is selected from the group consisting of: a cytokine, a chemokine, a homing molecule, a growth factor, a co-activation molecule, a tumor microenvironment modifier, a receptor, a ligand, an antibody, a peptide, and an enzyme.

24. The heterologous construct of claim 22, wherein each of the at least one effector molecule comprises:
a) a cytokine selected from the group consisting of: IL1-beta, IL2, IL4, IL6, IL7, IL10, IL12, an IL12p70 fusion protein, IL15, IL17A, IL18, IL21, IL22, Type I interferons, Interferon-gamma, and TNF-alpha;
b) a chemokine selected from the group consisting of: CCL21a, CXCL10, CXCL11, CXCL13, a CXCL10-CXCL11 fusion protein, CCL19, CXCL9, and XCL1;
c) a homing molecule selected from the group consisting of: anti-integrin alpha4, beta7; anti-MAdCAM; CCR9; CXCR4; SDFI; MMP-2; CXCR1; CXCR7; CCR2; CCR4; and GPR15;
d) a growth factor selected from the group consisting of: FLT3L and GM-CSF;
e) a co-activation molecule selected from the group consisting of: c-Jun, 4-1 BBL and CD40L; or
f) a tumor microenvironment modifier selected from the group consisting of: an adenosine deaminase, a TGFbeta inhibitor, an immune checkpoint inhibitor, a VEGF inhibitor, and an HPGE2.

25. The heterologous construct of claim 24, comprising a first effector molecule and a second effector molecule, wherein each of the first effector molecule and the second effector molecule is from a separate therapeutic class.

26. A vector comprising the heterologous construct of claim 20.

27. A dual expression vector comprising the heterologous construct of claim 20 and a second construct comprising a polynucleotide sequence encoding an activating immune receptor.

28. The dual expression vector of claim 27, wherein the activating immune receptor comprises an antigen recognizing receptor selected from a T Cell Receptor (TCR) or a Chimeric Antigen Receptor (CAR).

29. The dual expression vector of claim 28, wherein the TCR is an endogenous T cell receptor or an exogenous T cell receptor.

30. An immunoresponsive cell comprising the heterologous construct of claim 22.

31. The immunoresponsive cell of claim 30, wherein the immunoresponsive cell is selected from the group consisting of: a T cell, a CD8+ T cell, a CD4+ T cell, a gamma-delta T cell, a cytotoxic T lymphocyte (CTL), as regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

32. The immunoresponsive cell of claim 30, wherein the immunoresponsive cell expresses an activating immune receptor.

33. The immunoresponsive cell of claim 32, wherein the activating immune receptor comprises an antigen recognizing receptor.

34. The immunoresponsive cell of claim 33, wherein the antigen recognizing receptor comprises a T Cell Receptor (TCR) or a Chimeric Antigen Receptor (CAR).

35. The immunoresponsive cell of claim 34, wherein the TCR is an endogenous T cell receptor or an exogenous T cell receptor.

36. The immunoresponsive cell of claim 30, wherein the immunoresponsive cell is autologous or allogeneic.

37. A pharmaceutical composition comprising the engineered CCL3 promoter of claim 16, and a pharmaceutically acceptable carrier, pharmaceutically acceptable excipient, or a combination thereof.

38. A method of increasing expression of a target gene, the method comprising use of the engineered CCL3 promoter of claim 16 to increase expression of the target gene.

39. The method of claim 38, wherein the target gene is an immunomodulatory gene.

40. A method of treating a subject in need thereof, the method comprising administering the immunoresponsive cell of claim 30.

41. A method of stimulating a cell-mediated immune response in a subject, the method comprising administering to a subject the heterologous construct of claim 22.

42. A kit comprising the immunoresponsive cell of claim 30 and written instructions for using the immunoresponsive cell or pharmaceutical composition for treating and/or preventing the tumor in a subject.


SEQUENCE LISTING (282 sequences)

SEQ ID NO: 1 - DNA, 1221 bp, synthetic construct
  source 1..1221: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaatttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttagccctccacagcatcagcccatcaaccctatccctgtggttatagcagctgagttcatgcacagaccagttcttatgagaagggactgactaagaatagccttgggttgacatatacccctcttcacactcacaggattgcgtcaattcctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 2 - DNA, 1290 bp, synthetic construct
  source 1..1290: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaatttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggattgcgtcaattcctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 3 - DNA, 1825 bp, synthetic construct
  source 1..1825: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcaagtatagtataggatccttaaaatggttcaaagaaatgggaaatcaagacttcattttggccaaaaccattgaacagaaacttcagcatatttatcaactgacaacaacctatttttcaaccagtgatgttggaaatgtttttttaaaaattagtttataaatttgtgggctgaccaagaaggtaataaagtctaactaagtaaaatgagaaaaattcagtaagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgccacttcattcattttagagatctagtggataatttatcattccctgatggagaaaaatttaggttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttagccctccacagcatcagcccatcaaccctatccctgtggttatagcagctgagttcatgcacagaccagttcttatgagaagggactgactaagaatagccttgggttgacatatacccctcttcacactcacaggcctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 4 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggattgcgtcaattcctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 5 - DNA, 85 bp, synthetic construct
  source 1..85: mol_type = other DNA
ggacagaattccaaaggcatggtcgcacttggcttctgtcctctgttattctccagcatcaaatgtatcaactctaacccctttg

SEQ ID NO: 6 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 7 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcaccttacctactaggttaatccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 8 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcacccactcgaattcagacgacaaaaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 9 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccattctaagtccaattcacgacaagaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 10 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctcttcagactcaattattcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 11 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgtacacggttaagactcaatactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 12 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcgaaacgagattctgtacgactgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 13 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacatctacacgcattaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 14 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaatatctcgctaataggagtaagatacagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 15 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagcaagacctatactatccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 16 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgtgctatagtgctgtataactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 17 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcgtaccagaataaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 18 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatctatataccagcgagttcgataaaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 19 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagacgaagcaatacttaagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 20 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctcccgatagaaagaattgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 21 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgtctgtataaagctcgtaccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 22 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagaccatagtgaggaaattctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 23 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttaggttaagcatactaaacgttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 24 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgagtaaagaagcgaaatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 25 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttggtaaacctcaaatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 26 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccataaaacactaatttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 27 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttcgagcgacgcttaatattgctagatattttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 28 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccaccaaacgcaccagaactataaccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 29 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactcaagtctgatagtgagttgtctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 30 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttccgatctctagttaagttagctgtatagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 31 - DNA, 66 bp, synthetic construct
  source 1..66: mol_type = other DNA
atgtgtcaccagcagctcgttatatcctggtttagtttggtgtttctcgcttcacccctggtggca

SEQ ID NO: 32 - DNA, 66 bp, synthetic construct
  source 1..66: mol_type = other DNA
atgtgccatcagcaactcgtcatctcctggttctcccttgtgttcctcgcttcccctctggtcgcc

SEQ ID NO: 33 - DNA, 42 bp, synthetic construct
  source 1..42: mol_type = other DNA
atgcaactgctgtcatgtatcgcactcatcctggcgctggta

SEQ ID NO: 34 - DNA, 60 bp, synthetic construct
  source 1..60: mol_type = other DNA
atgtatcggatgcaacttttgagctgcatcgcattgtctctggcgctggtgacaaattcc

SEQ ID NO: 35 - DNA, 45 bp, synthetic construct
  source 1..45: mol_type = other DNA
atgaatctcttgctcatacttacgtttgtcgctgctgccgttgcg

SEQ ID NO: 36 - DNA, 51 bp, synthetic construct
  source 1..51: mol_type = other DNA
atgggcgtgaaggtcttgtttgcccttatctgcatagctgttgcggaggcg

SEQ ID NO: 37 - DNA, 72 bp, synthetic construct
  source 1..72: mol_type = other DNA
atgccgatggggagccttcaacctttggcaacgctttatcttctggggatgttggttgctagttgccttggg

SEQ ID NO: 38 - DNA, 63 bp, synthetic construct
  source 1..63: mol_type = other DNA
atggaaactgacacgttgttgctgtgggtattgctcttgtgggtcccaggatctacgggcgac

SEQ ID NO: 39 - DNA, 66 bp, synthetic construct
  source 1..66: mol_type = other DNA
atggatatgagggttcccgcccagcttttggggctgcttttgttgtggcttcgaggggctcggtgt

SEQ ID NO: 40 - DNA, 48 bp, synthetic construct
  source 1..48: mol_type = other DNA
atgaagtgtctgttgtacctggcgtttctgttcattggtgtaaactgt

SEQ ID NO: 41 - DNA, 84 bp, synthetic construct
  source 1..84: mol_type = other DNA
atgaatatcaaaggaagtccgtggaagggtagtctcctgctgctcctcgtatctaaccttctcctttgtcaatccgtggcaccc

SEQ ID NO: 42 - DNA, 54 bp, synthetic construct
  source 1..54: mol_type = other DNA
atgaaatgggtaacattcatatcacttctctttctgttcagctctgcgtattct

SEQ ID NO: 43 - DNA, 57 bp, synthetic construct
  source 1..57: mol_type = other DNA
atgacaaggcttactgttttggctctcctcgctggactcttggcttcctcccgagca

SEQ ID NO: 44 - DNA, 51 bp, synthetic construct
  source 1..51: mol_type = other DNA
atgagggcttggattttttttctgctctgccttgccggtcgagccctggcg

SEQ ID NO: 45 - DNA, 48 bp, synthetic construct
  source 1..48: mol_type = other DNA
atgcctcttctgcttttgcttcctcttttgtgggcaggtgccctcgca

SEQ ID NO: 46 - DNA, 87 bp, synthetic construct
  source 1..87: mol_type = other DNA
atgaactctttctcaacctctgcgtttggtccggtcgctttctcccttgggctcctgcttgtcttgccagcagcgtttcctgcgcca

SEQ ID NO: 47 - DNA, 60 bp, synthetic construct
  source 1..60: mol_type = other DNA
atgacaagtaaactggcggtagccttgctcgcggcctttttgatttccgcagccctttgt

SEQ ID NO: 48 - DNA, 69 bp, synthetic construct
  source 1..69: mol_type = other DNA
atgaaggtaagtgcagcgttgctttgccttctcctcattgcagcgacctttattcctcaagggctggcc

SEQ ID NO: 49 - DNA, 78 bp, synthetic construct
  source 1..78: mol_type = other DNA
atgggagcggcagctagaacacttcgacttgcccttgggctcttgctccttgcaaccctccttagacctgccgacgca

SEQ ID NO: 50 - DNA, 63 bp, synthetic construct
  source 1..63: mol_type = other DNA
atgtcaccgttgttgcggagattgctgttggccgcacttttgcaactggctcctgctcaagcc

SEQ ID NO: 51 - DNA, 63 bp, synthetic construct
  source 1..63: mol_type = other DNA
atgaataacctgctctgttgtgcgctcgtgttcctggacatttctataaaatggacaacgcaa

SEQ ID NO: 52 - DNA, 69 bp, synthetic construct
  source 1..69: mol_type = other DNA
atgcaaatgtctcctgcccttacctgtctcgtacttggtcttgcgctcgtatttggagagggatcagcc

SEQ ID NO: 53 - DNA, 102 bp, synthetic construct
  source 1..102: mol_type = other DNA
atggcaagggctgcactcagtgctgccccgtctaatcccagattgcttcgagttgcattgcttcttctgttgctggttgcagctggtaggagagcagcgggt

SEQ ID NO: 54 - DNA, 63 bp, synthetic construct
  source 1..63: mol_type = other DNA
atgaatgcaaaagtcgtggtcgtgctggttttggttctgacggcgttgtgtcttagtgatggg

SEQ ID NO: 55 - DNA, 66 bp, synthetic construct
  source 1..66: mol_type = other DNA
atggaacgcattgtgatctgcctgatggtcatcttcctgggcaccttagtgcacaagtcgagcagc

SEQ ID NO: 56 - protein, 22 aa, Homo sapiens
MCHQQLVISWFSLVFLASPLVA

SEQ ID NO: 57 - protein, 22 aa, synthetic construct
MCHQQLVISWFSLVFLASPLVA

SEQ ID NO: 58 - protein, 14 aa, synthetic construct
MQLLSCIALILALV

SEQ ID NO: 59 - protein, 20 aa, Homo sapiens
MYRMQLLSCIALSLALVTNS

SEQ ID NO: 60 - protein, 15 aa, Homo sapiens
MNLLLILTFVAAAVA

SEQ ID NO: 61 - protein, 17 aa, synthetic construct
MGVKVLFALICIAVAEA

SEQ ID NO: 62 - protein, 24 aa, Homo sapiens
MPMGSLQPLATLYLLGMLVASCLG

SEQ ID NO: 63 - protein, 21 aa, Mus musculus
METDTLLLWVLLLWVPGSTGD

SEQ ID NO: 64 - protein, 22 aa, Homo sapiens
MDMRVPAQLLGLLLLWLRGARC

SEQ ID NO: 65 - protein, 16 aa, synthetic construct
MKCLLYLAFLFIGVNC

SEQ ID NO: 66 - protein, 28 aa, Homo sapiens
MNIKGSPWKGSLLLLLVSNLLLCQSVAP

SEQ ID NO: 67 - protein, 18 aa, Homo sapiens
MKWVTFISLLFLFSSAYS

SEQ ID NO: 68 - protein, 19 aa, Homo sapiens
MTRLTVLALLAGLLASSRA

SEQ ID NO: 69 - protein, 17 aa, Homo sapiens
MRAWIFFLLCLAGRALA

SEQ ID NO: 70 - protein, 16 aa, Homo sapiens
MPLLLLLPLLWAGALA

SEQ ID NO: 71 - protein, 29 aa, Homo sapiens
MNSFSTSAFGPVAFSLGLLLVLPAAFPAP

SEQ ID NO: 72 - protein, 20 aa, Homo sapiens
MTSKLAVALLAAFLISAALC

SEQ ID NO: 73 - protein, 23 aa, Homo sapiens
MKVSAALLCLLLIAATFIPQGLA

SEQ ID NO: 74 - protein, 26 aa, Homo sapiens
MGAAARTLRLALGLLLLATLLRPADA

SEQ ID NO: 75 - protein, 21 aa, Homo sapiens
MSPLLRRLLLAALLQLAPAQA

SEQ ID NO: 76 - protein, 21 aa, Homo sapiens
MNNLLCCALVFLDISIKWTTQ

SEQ ID NO: 77 - protein, 23 aa, Homo sapiens
MQMSPALTCLVLGLALVFGEGSA

SEQ ID NO: 78 - protein, 34 aa, Homo sapiens
MARAALSAAPSNPRLLRVALLLLLLVAAGRRAAG

SEQ ID NO: 79 - protein, 21 aa, Homo sapiens
MNAKVVVVLVLVLTALCLSDG

SEQ ID NO: 80 - protein, 22 aa, Homo sapiens
MERIVICLMVIFLGTLVHKSSS

SEQ ID NO: 81 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatcgtagaactcttagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 82 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaataccttggtggagctcatctaatatcttcatattacctccaagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 83 - protein, 21 aa, Homo sapiens
MALPVTALLLPLALLLHAARP

SEQ ID NO: 84 - DNA, 63 bp, synthetic construct
  source 1..63: mol_type = other DNA
atggctctccctgtaactgccctgcttcttccccttgccttgcttctccatgccgctagaccc

SEQ ID NO: 85 - protein, 22 aa, Homo sapiens
MLLLVTSLLLCELPHPAFLLIP

SEQ ID NO: 86 - DNA, 66 bp, synthetic construct
  source 1..66: mol_type = other DNA
atgctgctgctggtcacatctctgctgctgtgcgagctgccccatcctgcctttctgctgatccct

SEQ ID NO: 87 - DNA, 66 bp, synthetic construct
  source 1..66: mol_type = other DNA
atgctgctgctggttacatctctgctgctgtgcgagctgccccatcctgcctttctgctgatccct

SEQ ID NO: 88 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgacgattgaacaaattgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 89 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacctataccttggttcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 90 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggattgcgtcaattcctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 91 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaaaaccacgtatctacttatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 92 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagccaaactctgctactcatcactatagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 93-95 - skipped (3 entries)

SEQ ID NO: 96 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgtgctagttgtccaataaagacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 97 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatcgacgtgtgtcatatagaataacatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 98-99 - skipped (2 entries)

SEQ ID NO: 100 - protein, 42 aa, synthetic construct
AAAIEVMYPPPYLDNEKSNGTIIHVKGKHLCPSPLFPGPSKP

SEQ ID NO: 101 - protein, 12 aa, synthetic construct
ESKYGPPCPSCP

SEQ ID NO: 102 - protein, 12 aa, synthetic construct
ESKYGPPAPSAP

SEQ ID NO: 103 - protein, 12 aa, synthetic construct
ESKYGPPCPPCP

SEQ ID NO: 104 - protein, 12 aa, synthetic construct
EPKSCDKTHTCP

SEQ ID NO: 105 - protein, 86 aa, synthetic construct
AAAFVPVFLPAKPTTTPAPRPPTPAPTIASQPLSLRPEACRPAAGGAVHTRGLDFACDIYIWAPLAGTCGVLLLSLVITLYCNHRN

SEQ ID NO: 106 - protein, 45 aa, synthetic construct
TTTPAPRPPTPAPTIALQPLSLRPEACRPAAGGAVHTRGLDFACD

SEQ ID NO: 107 - protein, 132 aa, synthetic construct
ACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEADAEC

SEQ ID NO: 108 - protein, 34 aa, synthetic construct
ACPTGLYTHSGECCKACNLGEGVAQPCGANQTVC

SEQ ID NO: 109 - protein, 20 aa, synthetic construct
AVGQDTQEVIVVPHSLPFKV

SEQ ID NO: 110 - DNA, 126 bp, synthetic construct
  source 1..126: mol_type = other DNA
gcagcagctatcgaggtgatgtatcctccgccctacctggataatgaaaagagtaatgggactatcattcatgtaaaagggaagcatctttgtccttctccccttttccccggtccgtctaaacct

SEQ ID NO: 111 - DNA, 36 bp, synthetic construct
  source 1..36: mol_type = other DNA
gaaagcaagtacggtccaccttgccctagctgtccg

SEQ ID NO: 112 - DNA, 36 bp, synthetic construct
  source 1..36: mol_type = other DNA
gaatccaagtacggccccccagcgcctagtgcccca

SEQ ID NO: 113 - DNA, 36 bp, synthetic construct
  source 1..36: mol_type = other DNA
gaatctaaatatggcccgccatgcccgccttgccca

SEQ ID NO: 114 - DNA, 36 bp, synthetic construct
  source 1..36: mol_type = other DNA
gaaccgaagtcttgtgataaaactcatacgtgcccg

SEQ ID NO: 115 - DNA, 258 bp, synthetic construct
  source 1..258: mol_type = other DNA
gctgctgctttcgtacccgtgttcctccctgctaagcctacgactacccccgcaccgagaccacccacgccagcacccacgattgctagccagccccttagtttgcgaccagaagcttgtcggcctgctgctggtggcgcggtacatacccgcggccttgattttgcttgcgatatatatatctgggcgcctctggccggaacatgcggggtcctcctcctttctctggttattactctctactgtaatcacaggaat

SEQ ID NO: 116 - DNA, 396 bp, synthetic construct
  source 1..396: mol_type = other DNA
gcctgcccgaccgggctctacactcatagcggggaatgttgtaaggcatgtaacttgggtgagggcgtcgcacagccctgcggagctaaccaaacagtgtgcgaaccctgcctcgatagtgtgacgttctctgatgttgtatcagctacagagccttgcaaaccatgtactgagtgcgttggacttcagtcaatgagcgctccatgtgtggaggcagatgatgcggtctgtcgatgtgcttacggatactaccaagacgagacaacagggcggtgcgaggcctgtagagtttgtgaggcgggctccgggctggtgttttcatgtcaagacaagcaaaatacggtctgtgaagagtgccctgatggcacctactcagacgaagcagatgcagaatgc

SEQ ID NO: 117 - DNA, 102 bp, synthetic construct
  source 1..102: mol_type = other DNA
gcctgccctacaggactctacacgcatagcggtgagtgttgtaaagcatgcaacctcggggaaggtgtagcccagccatgcggggctaaccaaaccgtttgc

SEQ ID NO: 118 - DNA, 60 bp, synthetic construct
  source 1..60: mol_type = other DNA
gctgtgggccaggacacgcaggaggtcatcgtggtgccacactccttgccctttaaggtg

SEQ ID NO: 119 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccattttacaaccctatagggctgcctataaagaggagagctggtttcagacttcagaaggacacgggcagcagacagtggtcagtcctttcttggctctgctgacactcgagcccacattccgtcacctgctcagaatc

SEQ ID NO: 120 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttctagtctaagttgacactcgagcccacattccgtcacctgctcagaatc

SEQ ID NO: 121 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattggaagtagcttgtttaatc

SEQ ID NO: 122 - DNA, 2085 bp, synthetic construct
  source 1..2085: mol_type = other DNA
ggacagaattccaaaggcatggtcgcacttggcttctgtcctctgttattctccagcatcaaatgtatcaactctaacccctttgcgtagaacttcgtaacattaagtctcgtagataacgccgtcattgtatttctctaacgaccccactgctctgcagctttctcagctaatatcctctttcctctgaacccaccaagctatggcactatgatagtgactattatactaacagtactgtagggcaatagtctggttgtcgctattccatatcgagtgaatctatcccttccatgaagccttccccaacttacaggtggaaggtcacaactaacccataaccgtgttagagctttcatttctttttctaagtttgtgagtgtacgaacatgctaaatgctacaattactttatattagttcatcaccatttgtgtcttactctaacttagagcgaagtaaagatcattaaaatcgttctgaatggactacacatatacaactcgaaagacttcgcactaagccgtctgtatattaacaaagtatggaaaagaaaaaaaattgacatgctctgtagaaaaggtagagacccttttatccgctatccagatagataccaaaagaataagtaaaaaataggaatgctatagcatgatttatgtacattctcttcgacggactatcatgcaaggtgtaaggtagcgatctaagacgtttgtatcgaactaagtagacattcttgtgtatatgttagagcataataatttctttcagattaaactttatcctttgcgcactttgagggtctatttacacgattaatcttgtcgactgctcatacaacagcataatgcgactacgtcgattacagaagtcttggacattactttcgcttaaatatatcgaaaaagaaaaaaaactagcttcgcgtaaagcactgaccaattggtgttgtatgtaatatcggaccaagatgacaagtatactcataacgtattgtatttttttccatgattcgtacatgatggttatacagtacgtttactgtagcacactaaataacttaagatcgaaaccgagtgatacgtacttaagtacgcaacgcttaccgtctacagcattgagaattatcctactgcatctaaagtcgctatgctacgactggtaagttaagttataagcatatttatgtttctagagttgaatgtctcacttaataataaatgaagagttacatcgaaatcggcattatgatgttccacacatatcctttgtttattttagaaaatgacgatatgctgggtcaggttatgcgtttcttgaagatggctccaattttgggttgtttccacagaactctttcctagaaatgctgtcggtatctattacgagaagcctgatcaatttcggcttatttaaccaatcctcaatctaaagtatttttatgatgccgttcagatcgatctatagaccatccgaattgaagatactgatgataactatcggtaaatgaaacatgacctcatgcttttctcaagctgttgagatcgtgaacaaattgcctctgggaggaggaagggttgtgcactctcgaatagtgtcctatgagagctttcaatcctgtggttatcgagatataggaagcagaattgcagctctgtgggaaggaatggggctggagtacaaggactcggtaactacgcagaatatagctgtatactaagaatagcctttacagacagctaaagcctcttcacactagatatagaaaccatttcagttacatatagaagtataaagtagcccaatgagcccttgatcgaactacgtgaacagatgctattcttggagaagttgagaacctgtggtcaccagggaccctgagttgtgcaacttagcatgacttacggagtcatacgtttgttttccctcctcacccccagattcttcgtccccatccgcctgtattccctataaagaggagagttactcttctgatatcccttatccgaattccaatgggactaccttccaacaattggctctgcatatgagacctacaaaccaccgtcacctgctcagaata

SEQ ID NO: 123 - DNA, 1306 bp, synthetic construct
  source 1..1306: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaatttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttagccctccacagcatcagcccatcaaccctatccctgtggttatagcagctgagttcatgcacagaccagttcttatgagaagggactgactaagaatagccttgggttgacatatacccctcttcacactcacaggattgcgtcaattcctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 124 - DNA, 1375 bp, synthetic construct
  source 1..1375: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaatttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggattgcgtcaattcctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 125 - DNA, 1910 bp, synthetic construct
  source 1..1910: mol_type = other DNA
ggacagaattccaaaggcatggtcgcacttggcttctgtcctctgttattctccagcatcaaatgtatcaactctaacccctttggggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcaagtatagtataggatccttaaaatggttcaaagaaatgggaaatcaagacttcattttggccaaaaccattgaacagaaacttcagcatatttatcaactgacaacaacctatttttcaaccagtgatgttggaaatgtttttttaaaaattagtttataaatttgtgggctgaccaagaaggtaataaagtctaactaagtaaaatgagaaaaattcagtaagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgccacttcattcattttagagatctagtggataatttatcattccctgatggagaaaaatttaggttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttagccctccacagcatcagcccatcaaccctatccctgtggttatagcagctgagttcatgcacagaccagttcttatgagaagggactgactaagaatagccttgggttgacatatacccctcttcacactcacaggcctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 126 - DNA, 2085 bp, synthetic construct; identical to SEQ ID NO: 90
  source 1..2085: mol_type = other DNA

SEQ ID NO: 127-131 - skipped (5 entries)

SEQ ID NO: 132 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 133 - DNA, 2000 bp, synthetic construct
  source 1..2000: mol_type = other DNA
gggggaatacaaggcctgtcctggtttggtcccaatttagctttatcatccatattcaccttacctactaggttaatccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 134 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcacccactcgaattcagacgacaaaaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 135 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccattctaagtccaattcacgacaagaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 136 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctcttcagactcaattattcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 137 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgtacacggttaagactcaatactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 138 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcgaaacgagattctgtacgactgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 139 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacatctacacgcattaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 140 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaatatctcgctaataggagtaagatacagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 141 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagcaagacctatactatccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 142 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgtgctatagtgctgtataactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 143 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcgtaccagaataaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 144 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatctatataccagcgagttcgataaaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 145 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagacgaagcaatacttaagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 146 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctcccgatagaaagaattgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 147 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgtctgtataaagctcgtaccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 148 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagaccatagtgaggaaattctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 149 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttaggttaagcatactaaacgttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 150 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgagtaaagaagcgaaatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 151 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttggtaaacctcaaatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 152 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccataaaacactaatttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 153 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttcgagcgacgcttaatattgctagatattttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 154 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccaccaaacgcaccagaactataaccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 155 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactcaagtctgatagtgagttgtctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 156 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttccgatctctagttaagttagctgtatagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 157 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatcgtagaactcttagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 158 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaataccttggtggagctcatctaatatcttcatattacctccaagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 159 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgacgattgaacaaattgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 160 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacctataccttggttcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 161 - DNA, 2000 bp, synthetic construct; identical to SEQ ID NO: 4
  source 1..2000: mol_type = other DNA

SEQ ID NO: 162 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaaaaccacgtatctacttatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 163 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagccaaactctgctactcatcactatagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 164 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgtgctagttgtccaataaagacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 165 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatcgacgtgtgtcatatagaataacatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 166 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccattttacaaccctatagggctgcctataaagaggagagctggtttcagacttcagaaggacacgggcagcagacagtggtcagtcctttcttggctctgctgacactcgagcccacattccgtcacctgctcagaatc

SEQ ID NO: 167 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttctagtctaagttgacactcgagcccacattccgtcacctgctcagaatc

SEQ ID NO: 168 - DNA, 2000 bp, synthetic construct
  source 1..2000: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaaactgcttcaatttggcatgatttatgtaagtatagtataggatccttaaaatggttcaaagaaatgggaaatcaagacttcattttggccaaaaccattgaacagaaacttcagcatatttatcaataatttctttcagattaaacaactgacaacaacctatttttcaaccagtgatgttggaaatgtttttttaaaaattagtttataaatttgtgggctgaccaagaaggtaataaagtctaactaagtaaaatgagaaaaattcagaaaaagaaaaaaataagaaaataaatcacccagggacctatcacacaaatataagaactattcattctttaaggcatgtatttccaagcctttgtatttttttccatgcttagggttggcaaggaatatatatatatttgtacaaatatatatgtgtatatgtacaaatacatgtatatatagtacaaatatatatatatatttgtacaattcttcagactttgtagaatttgtataatgtcgtatcttgctttttttaaccactgatgttataagcatatttatgccacttcattcattttagagacttaataataaatgatctagtggataatttatcattccctgatggagaaaaatttagctttgtttattttagagttataaacgatgctgggtcaggtatctttatgtttgaagatggctccatatttgggttgtttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgaggaagcagaattgcagctctgtgggaaggaatggggctggagagttcatgcacagaccagttcttatgagaagggactgactaagaatagccttgggttgacatatacccctcttcacactcacaggagaaaccatttccctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattggaagtagcttgtttaatc

SEQ ID NO: 169 - DNA, 2000 bp, synthetic construct
  source 1..2000: mol_type = other DNA
cgtagaacttcgtaacattaagtctcgtagataacgccgtcattgtatttctctaacgaccccactgctctgcagctttctcagctaatatcctctttcctctgaacccaccaagctatggcactatgatagtgactattatactaacagtactgtagggcaatagtctggttgtcgctattccatatcgagtgaatctatcccttccatgaagccttccccaacttacaggtggaaggtcacaactaacccataaccgtgttagagctttcatttctttttctaagtttgtgagtgtacgaacatgctaaatgctacaattactttatattagttcatcaccatttgtgtcttactctaacttagagcgaagtaaagatcattaaaatcgttctgaatggactacacatatacaactcgaaagacttcgcactaagccgtctgtatattaacaaagtatggaaaagaaaaaaaattgacatgctctgtagaaaaggtagagacccttttatccgctatccagatagataccaaaagaataagtaaaaaataggaatgctatagcatgatttatgtacattctcttcgacggactatcatgcaaggtgtaaggtagcgatctaagacgtttgtatcgaactaagtagacattcttgtgtatatgttagagcataataatttctttcagattaaactttatcctttgcgcactttgagggtctatttacacgattaatcttgtcgactgctcatacaacagcataatgcgactacgtcgattacagaagtcttggacattactttcgcttaaatatatcgaaaaagaaaaaaaactagcttcgcgtaaagcactgaccaattggtgttgtatgtaatatcggaccaagatgacaagtatactcataacgtattgtatttttttccatgattcgtacatgatggttatacagtacgtttactgtagcacactaaataacttaagatcgaaaccgagtgatacgtacttaagtacgcaacgcttaccgtctacagcattgagaattatcctactgcatctaaagtcgctatgctacgactggtaagttaagttataagcatatttatgtttctagagttgaatgtctcacttaataataaatgaagagttacatcgaaatcggcattatgatgttccacacatatcctttgtttattttagaaaatgacgatatgctgggtcaggttatgcgtttcttgaagatggctccaattttgggttgtttccacagaactctttcctagaaatgctgtcggtatctattacgagaagcctgatcaatttcggcttatttaaccaatcctcaatctaaagtatttttatgatgccgttcagatcgatctatagaccatccgaattgaagatactgatgataactatcggtaaatgaaacatgacctcatgcttttctcaagctgttgagatcgtgaacaaattgcctctgggaggaggaagggttgtgcactctcgaatagtgtcctatgagagctttcaatcctgtggttatcgagatataggaagcagaattgcagctctgtgggaaggaatggggctggagtacaaggactcggtaactacgcagaatatagctgtatactaagaatagcctttacagacagctaaagcctcttcacactagatatagaaaccatttcagttacatatagaagtataaagtagcccaatgagcccttgatcgaactacgtgaacagatgctattcttggagaagttgagaacctgtggtcaccagggaccctgagttgtgcaacttagcatgacttacggagtcatacgtttgttttccctcctcacccccagattcttcgtccccatccgcctgtattccctataaagaggagagttactcttctgatatcccttatccgaattccaatgggactaccttccaacaattggctctgcatatgagacctacaaaccaccgtcacctgctcagaata

SEQ ID NO: 170 - DNA, 18 bp, synthetic construct
  source 1..18: mol_type = other DNA
agctgcagagcagtgggg

SEQ ID NO: 171 - DNA, 18 bp, synthetic construct
  source 1..18: mol_type = other DNA
cttacctactaggttaat

SEQ ID NO: 172 - DNA, 20 bp, synthetic construct
  source 1..20: mol_type = other DNA
cctctttcctctgaacccac

SEQ ID NO: 173 - DNA, 20 bp, synthetic construct
  source 1..20: mol_type = other DNA
actcgaattcagacgacaaa

SEQ ID NO: 174 - DNA, 24 bp, synthetic construct
  source 1..24: mol_type = other DNA
ttggggaaggcttcatggaaggga

SEQ ID NO: 175 - DNA, 23 bp, synthetic construct
  source 1..23: mol_type = other DNA
ttctaagtccaattcacgacaag

SEQ ID NO: 176 - DNA, 13 bp, synthetic construct
  source 1..13: mol_type = other DNA
ggtggaaggtcac

SEQ ID NO: 177 - DNA, 13 bp, synthetic construct
  source 1..13: mol_type = other DNA
tcagactcaatta

SEQ ID NO: 178 - DNA, 20 bp, synthetic construct
  source 1..20: mol_type = other DNA
gagctttcatttctttttct

SEQ ID NO: 179 - DNA, 20 bp, synthetic construct
  source 1..20: mol_type = other DNA
gtacacggttaagactcaat

SEQ ID NO: 180 - DNA, 18 bp, synthetic construct
  source 1..18: mol_type = other DNA
gctaaatgctacaattac

SEQ ID NO: 181 - DNA, 18 bp, synthetic construct
  source 1..18: mol_type = other DNA
aaacgagattctgtacga

SEQ ID NO: 182 - DNA, 13 bp, synthetic construct
  source 1..13: mol_type = other DNA
ttttaatgatctt

SEQ ID NO: 183 - DNA, 13 bp, synthetic construct
  source 1..13: mol_type = other DNA
tctacacgcatta

SEQ ID NO: 184 - DNA, 24 bp, synthetic construct
  source 1..24: mol_type = other DNA
acaaagtatggaaaagaaaaaaaa

SEQ ID NO: 185 - DNA, 24 bp, synthetic construct
  source 1..24: mol_type = other DNA
tctcgctaataggagtaagataca

SEQ ID NO: 186 - DNA, 14 bp, synthetic construct
  source 1..14: mol_type = other DNA
aaggtagagaccct

SEQ ID NO: 187 - DNA, 14 bp, synthetic construct
  source 1..14: mol_type = other DNA
caagacctatacta

SEQ ID NO: 188 - DNA, 17 bp, synthetic construct
  source 1..17: mol_type = other DNA
tttttacttattctttt

SEQ ID NO: 189 - DNA, 17 bp, synthetic construct
  source 1..17: mol_type = other DNA
tgctatagtgctgtata

SEQ ID NO: 190 - DNA, 11 bp, synthetic construct
  source 1..11: mol_type = other DNA
atgatttatgt

SEQ ID NO: 191 - DNA, 11 bp, synthetic construct
  source 1..11: mol_type = other DNA
gtaccagaata

SEQ ID NO: 192 - DNA, 22 bp, synthetic construct
  source 1..22: mol_type = other DNA
gtttaatctgaaagaaattatt

SEQ ID NO: 193 - DNA, 22 bp, synthetic construct
  source 1..22: mol_type = other DNA
tatataccagcgagttcgataa

SEQ ID NO: 194 - DNA, 13 bp, synthetic construct
  source 1..13: mol_type = other DNA
aaaaagaaaaaaa

SEQ ID NO: 195 - DNA, 13 bp, synthetic construct
  source 1..13: mol_type = other DNA
acgaagcaatact

SEQ ID NO: 196 - DNA, 16 bp, synthetic construct
  source 1..16: mol_type = other DNA
ttgtatttttttccat

SEQ ID NO: 197 - DNA, 16 bp, synthetic construct
  source 1..16: mol_type = other DNA
cccgatagaaagaatt

SEQ ID NO: 198 - DNA, 17 bp, synthetic construct
  source 1..17: mol_type = other DNA
ttataagcatatttatg

SEQ ID NO: 199 - DNA, 17 bp, synthetic construct
  source 1..17: mol_type = other DNA
tctgtataaagctcgta

SEQ ID NO: 200 - DNA, 15 bp, synthetic construct
  source 1..15: mol_type = other DNA
tcatttattattaag

SEQ ID NO: 201 - DNA, 15 bp, synthetic construct
  source 1..15: mol_type = other DNA
ccatagtgaggaaat

SEQ ID NO: 202 - DNA, 16 bp, synthetic construct
  source 1..16: mol_type = other DNA
ctttgtttattttaga

SEQ ID NO: 203 - DNA, 16 bp, synthetic construct
  source 1..16: mol_type = other DNA
gttaagcatactaaac

SEQ ID NO: 204 - DNA, 13 bp, synthetic construct
  source 1..13: mol_type = other DNA
tgctgggtcaggt

SEQ ID NO: 205 - DNA, 13 bp, synthetic construct
  source 1..13: mol_type = other DNA
gtaaagaagcgaa

SEQ ID NO: 206 - DNA, 13 bp, synthetic construct
  source 1..13: mol_type = other DNA
gaagatggctcca

SEQ ID NO: 207 - DNA, 13 bp, synthetic construct
  source 1..13: mol_type = other DNA
ggtaaacctcaaa

SEQ ID NO: 208-209 - skipped (2 entries)

SEQ ID NO: 210 - DNA, 29 bp, synthetic construct
  source 1..29: mol_type = other DNA
tttccacagaactctttcctagaaatgct

SEQ ID NO: 211 - DNA, 29 bp, synthetic construct
  source 1..29: mol_type = other DNA
tttcgagcgacgcttaatattgctagata

SEQ ID NO: 212 - DNA, 19 bp, synthetic construct
  source 1..19: mol_type = other DNA
tctaaagtatttttatgat

SEQ ID NO: 213 - DNA, 19 bp, synthetic construct
  source 1..19: mol_type = other DNA
aaacgcaccagaactataa

SEQ ID NO: 214 - DNA, 21 bp, synthetic construct
  source 1..21: mol_type = other DNA
tgaaacatgacctcatgcttt

SEQ ID NO: 215 - DNA, 21 bp, synthetic construct
  source 1..21: mol_type = other DNA
tcaagtctgatagtgagttgt

SEQ ID NO: 216 - DNA, 26 bp, synthetic construct
  source 1..26: mol_type = other DNA
gcctctgggaggaggaagggttgtgc

SEQ ID NO: 217 - DNA, 26 bp, synthetic construct
  source 1..26: mol_type = other DNA
ccgatctctagttaagttagctgtat

SEQ ID NO: 218 - DNA, 11 bp, synthetic construct
  source 1..11: mol_type = other DNA
cctgtggttat

SEQ ID NO: 219 - DNA, 11 bp, synthetic construct
  source 1..11: mol_type = other DNA
gtagaactctt

SEQ ID NO: 220 - DNA, 43 bp, synthetic construct
  source 1..43: mol_type = other DNA
aggaagcagaattgcagctctgtgggaaggaatggggctggag

SEQ ID NO: 221 - DNA, 43 bp, synthetic construct
  source 1..43: mol_type = other DNA
aataccttggtggagctcatctaatatcttcatattacctcca

SEQ ID NO: 222 - DNA, 12 bp, synthetic construct
  source 1..12: mol_type = other DNA
ggctattcttag

SEQ ID NO: 223 - DNA, 12 bp, synthetic construct
  source 1..12: mol_type = other DNA
cgattgaacaaa

SEQ ID NO: 224 - DNA, 12 bp, synthetic construct
  source 1..12: mol_type = other DNA
agtgtgaagagg

SEQ ID NO: 225 - DNA, 12 bp, synthetic construct
  source 1..12: mol_type = other DNA
tataccttggtt

SEQ ID NO: 226 - DNA, 12 bp, synthetic construct
  source 1..12: mol_type = other DNA
gaaatggtttct

SEQ ID NO: 227 - DNA, 12 bp, synthetic construct
  source 1..12: mol_type = other DNA
attgcgtcaatt

SEQ ID NO: 228 - DNA, 15 bp, synthetic construct
  source 1..15: mol_type = other DNA
gatgctattcttgga

SEQ ID NO: 229 - DNA, 15 bp, synthetic construct
  source 1..15: mol_type = other DNA
aaccacgtatctact

SEQ ID NO: 230 - DNA, 22 bp, synthetic construct
  source 1..22: mol_type = other DNA
cctgtggtcaccagggaccctg

SEQ ID NO: 231 - DNA, 22 bp, synthetic construct
  source 1..22: mol_type = other DNA
aaactctgctactcatcactat

SEQ ID NO: 232 - DNA, 21 bp, synthetic construct
  source 1..21: mol_type = other DNA
agttgtgcaacttagcatgac

SEQ ID NO: 233 - DNA, 21 bp, synthetic construct
  source 1..21: mol_type = other DNA
tgctagttgtccaataaagac

SEQ ID NO: 234 - DNA, 22 bp, synthetic construct
  source 1..22: mol_type = other DNA
gaatctgggggtgaggagggaa

SEQ ID NO: 235 - DNA, 22 bp, synthetic construct
  source 1..22: mol_type = other DNA
cgacgtgtgtcatatagaataa

SEQ ID NO: 236 - DNA, 11 bp, synthetic construct
  source 1..11: mol_type = other DNA
ggcggatgggg

SEQ ID NO: 237 - DNA, 11 bp, synthetic construct
  source 1..11: mol_type = other DNA
tacaaccctat

SEQ ID NO: 238-239 - skipped (2 entries)

SEQ ID NO: 240 - DNA, 17 bp, synthetic construct
  source 1..17: mol_type = other DNA
ttctgagcaggtgacgg

SEQ ID NO: 241 - DNA, 17 bp, synthetic construct
  source 1..17: mol_type = other DNA
ggaagtagcttgtttaa

SEQ ID NO: 242 - DNA, 1188 bp, synthetic construct
  source 1..1188: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaatttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgacctattcctccagctttcattcagttctttagccctccacagcatcagcccatcaaccctatccctgtggttatagcagctgagttcatgcacagaccagttcttatgagaagggactgactaagaatagccttgggttgacatatacccacaggattgcgtcaattcctatgaaactataacaagtcatgagttgagagctgagagttagagaatagctcaaagatgctattcttggatatcctgagcccctgtggtcaccagggaccctgagttgtgcaacttagcatgacagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 243 - DNA, 1166 bp, synthetic construct
  source 1..1166: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaatttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgactgaaacatgacctcatgctttctattcctccagctttcattcagttctttgcctctgggaggaggaagggttgtgcagccctccacagcatcagcccatcaaccctatccctgtggttatagcagctgagttcatgcacagaccagttcttatgagaagggactgactaagaatagccttgggttgacatatacccacaggattgcgtcaattcctatgaaactataacaagtcatgagttgagagctgagagttagagaatagctcaaaagcatcactacgcttaaaaatttccctcctcacccccagattccatttccccatccgccagggctgcctataaagaggagagctggtttcagacttcagaaggacacgggcagcagacagtggtcagtcctttcttggctctgctgacactcgagcccacattccgtcacctgctcagaatc

SEQ ID NO: 244 - DNA, 956 bp, synthetic construct
  source 1..956: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaatttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgacagccctccacagcatcagcccatcaaccctatccctgtggttatagcagctgagttcatgcacagaccagttcttatgagaagggactgactaagaatagccttgggttgacatatacccacaggattgcgtcaattcctatgaaactataacaagtcatgagttgagagctgagagttagagaatagctcaaaagcatcactacgcttaaaaatttccctcctcacccccagattccattt

SEQ ID NO: 245 - DNA, 881 bp, synthetic construct
  source 1..881: mol_type = other DNA
gggggaatacaaggcctgtcctggtttggtcccaatttagctttatcatccatattcacccccactgctctgcagctccactgaagcaccccctctttcctctgaacccacaatgtcacactcaggactctgcctcagctgggcactcatctatagatgcctaaatcccgggcagttatccagacacaactaaagttccatcccttccatgaagccttccccaaccctctggtggaaggtcacttcttcccctcgtgggattctgagctttcatttctttttctactaggagtcctagcactttcggctaaatgctacaattacctgttcatacactctacctgcccccacgagatcaggggcatctcagaaacaaagatcattaaaaccaactaaatctatttctcattataaaatgaggtatgctgattgattgtgaaagaataaaataacaaagtatggaaaagaaaaaaaagcatataatctggctgagaaggtagagacccttccacaccactgaaattatgtattgaaaagaataagtaaaaatttccacagaactctttcctagaaatgctttttctaggttaatggctacagatatttctaggcacctgacatattgacacccacctctaaagtatttttatgatccacaactagcgtttaacacagcgccctagtcactacatgactaataaatagacaaatgacagccctccacagcatcagcccatcaaccctatccctgtggttatagcagctgagttcatgcacagaccagttcttatgagaagggactgactaagaatagccttgggttgacatataccagcatcactacgcttaaaaatttccctcctcacccccagattccattt

SEQ ID NO: 246 - DNA, 274 bp, synthetic construct
  source 1..274: mol_type = other DNA
ccccactgctctgcagctcctctttcctctgaacccactcccttccatgaagccttccccaaggtggaaggtcacgagctttcatttctttttctgctaaatgctacaattacaagatcattaaaaacaaagtatggaaaagaaaaaaaaaaggtagagaccctaaaagaataagtaaaaatttccacagaactctttcctagaaatgcttctaaagtatttttatgatcctgtggttatctaagaatagccttccctcctcacccccagattc

SEQ ID NO: 247 - DNA, 18 bp, synthetic construct
  source 1..18: mol_type = other DNA
ccccactgctctgcagct

SEQ ID NO: 248 - DNA, 20 bp, synthetic construct
  source 1..20: mol_type = other DNA
gtgggttcagaggaaagagg

SEQ ID NO: 249 - DNA, 24 bp, synthetic construct
  source 1..24: mol_type = other DNA
tcccttccatgaagccttccccaa

SEQ ID NO: 250 - DNA, 13 bp, synthetic construct
  source 1..13: mol_type = other DNA
gtgaccttccacc

SEQ ID NO: 251 - DNA, 20 bp, synthetic construct
  source 1..20: mol_type = other DNA
agaaaaagaaatgaaagctc

SEQ ID NO: 252 - DNA, 18 bp, synthetic construct
  source 1..18: mol_type = other DNA
gtaattgtagcatttagc

SEQ ID NO: 253 - DNA, 13 bp, synthetic construct
  source 1..13: mol_type = other DNA
aagatcattaaaa

SEQ ID NO: 254 - DNA, 24 bp, synthetic construct
  source 1..24: mol_type = other DNA
ttttttttcttttccatactttgt

SEQ ID NO: 255 - DNA, 14 bp, synthetic construct
  source 1..14: mol_type = other DNA
agggtctctacctt

SEQ ID NO: 256 - DNA, 17 bp, synthetic construct
  source 1..17: mol_type = other DNA
aaaagaataagtaaaaa

SEQ ID NO: 257 - DNA, 11 bp, synthetic construct
  source 1..11: mol_type = other DNA
acataaatcat

SEQ ID NO: 258 - DNA, 22 bp, synthetic construct
  source 1..22: mol_type = other DNA
aataatttctttcagattaaac

SEQ ID NO: 259 - DNA, 13 bp, synthetic construct
  source 1..13: mol_type = other DNA
tttttttcttttt

SEQ ID NO: 260 - DNA, 16 bp, synthetic construct
  source 1..16: mol_type = other DNA
atggaaaaaaatacaa

SEQ ID NO: 261 - DNA, 17 bp, synthetic construct
  source 1..17: mol_type = other DNA
cataaatatgcttataa

SEQ ID NO: 262 - DNA, 15 bp, synthetic construct
  source 1..15: mol_type = other DNA
cttaataataaatga

SEQ ID NO: 263 - DNA, 16 bp, synthetic construct
  source 1..16: mol_type = other DNA
tctaaaataaacaaag

SEQ ID NO: 264 - DNA, 13 bp, synthetic construct
  source 1..13: mol_type = other DNA
acctgacccagca

SEQ ID NO: 265 - DNA, 13 bp, synthetic construct
  source 1..13: mol_type = other DNA
tggagccatcttc

SEQ ID NO: 266 - skipped

SEQ ID NO: 267 - DNA, 29 bp, synthetic construct
  source 1..29: mol_type = other DNA
agcatttctaggaaagagttctgtggaaa

SEQ ID NO: 268 - DNA, 19 bp, synthetic construct
  source 1..19: mol_type = other DNA
atcataaaaatactttaga

SEQ ID NO: 269 - DNA, 21 bp, synthetic construct
  source 1..21: mol_type = other DNA
aaagcatgaggtcatgtttca

SEQ ID NO: 270 - DNA, 26 bp, synthetic construct
  source 1..26: mol_type = other DNA
gcacaacccttcctcctcccagaggc

SEQ ID NO: 271 - DNA, 11 bp, synthetic construct
  source 1..11: mol_type = other DNA
ataaccacagg

SEQ ID NO: 272 - DNA, 43 bp, synthetic construct
  source 1..43: mol_type = other DNA
ctccagccccattccttcccacagagctgcaattctgcttcct

SEQ ID NO: 273 - DNA, 12 bp, synthetic construct
  source 1..12: mol_type = other DNA
ctaagaatagcc

SEQ ID NO: 274 - DNA, 12 bp, synthetic construct
  source 1..12: mol_type = other DNA
cctcttcacact

SEQ ID NO: 275 - DNA, 12 bp, synthetic construct
  source 1..12: mol_type = other DNA
agaaaccatttc

SEQ ID NO: 276 - DNA, 15 bp, synthetic construct
  source 1..15: mol_type = other DNA
tccaagaatagcatc

SEQ ID NO: 277 - DNA, 22 bp, synthetic construct
  source 1..22: mol_type = other DNA
cagggtccctggtgaccacagg

SEQ ID NO: 278 - DNA, 21 bp, synthetic construct
  source 1..21: mol_type = other DNA
gtcatgctaagttgcacaact

SEQ ID NO: 279 - DNA, 22 bp, synthetic construct
  source 1..22: mol_type = other DNA
ttccctcctcacccccagattc

SEQ ID NO: 280 - DNA, 11 bp, synthetic construct
  source 1..11: mol_type = other DNA
ccccatccgcc

SEQ ID NO: 281 - skipped

SEQ ID NO: 282 - DNA, 17 bp, synthetic construct
  source 1..17: mol_type = other DNA
ccgtcacctgctcagaa